(19) United States
(12) Patent Application Publication
(10) Pub. No.: US 2026/0007640 A1
(43) Pub. Date: Jan. 8, 2026

(54) COMPOSITIONS FOR THE TREATMENT OF INFLAMMATORY DISORDERS

(71) Applicant: University of Tennessee Research Foundation, Memphis, TN (US)

(72) Inventors: Duane Miller, Collierville, TN (US); Udai Singh, Memphis, TN (US); Dong-Jin Hwang, Arlington, TN (US); Mousumi Mandal, Memphis, TN (US); Ahmed Rakib, Memphis, TN (US); Md Abdullah Al Mamun, Memphis, TN (US)

(73) Assignee: University of Tennessee Research Foundation, Memphis, TN (US)

(21) Appl. No.: 19/206,442

(22) Filed: May 13, 2025

Related U.S. Application Data
(60) Provisional application No. 63/646,308, filed on May 13, 2024.

(51) Int. Cl. A61K 31/426; A61P 1/00; A61P 29/00; C07D 277/24

(52) U.S. Cl. CPC A61K 31/426; A61P 1/00; A61P 29/00; C07D 277/24

(57) ABSTRACT

The present invention disclosures compounds and methods of use thereof for the treatment of an inflammatory disorder or disease in an individual in need thereof, wherein for example a compound or a pharmaceutically acceptable salt or prodrug thereof, having the chemical structure of Formula (I) or Formula (II) is administered to a patient.

### STATEMENT OF FEDERALLY SPONSORED RESEARCH

This invention was made with government support under grant number R01AI140405 awarded by National Institutes of Health. The government has certain rights in the invention.

### TECHNICAL FIELD

This invention relates generally to the field of therapeutics for treatment of diseases, and particularly diseases that involve responses or activation of the immune system. This invention further relates to the field of treatment of inflammatory disorders and diseases using a compound of the formula:

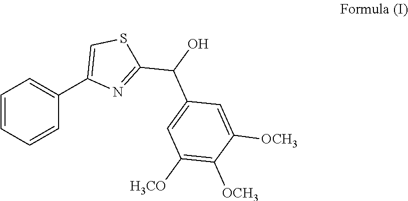 [SMILES: COc1cc(C(O)c2nc(-c3ccccc3)cs2)cc(OC)c1OC]

or a pharmaceutical composition thereof.

### BACKGROUND

Inflammation, an evolutionarily conserved process, is the mechanism of the body's defense program against various pathogens, and it generally terminates promptly. However, prolonged and higher magnitude of inflammation causes tissue and organ damage, delayed wound healing, and paves the way for diseases like cardiovascular disease, and cancer. Notably, chronic inflammation elicits the possibility of several diseases such as rheumatoid arthritis, multiple sclerosis, systemic lupus erythematosus, type 1 diabetes (T1D), psoriasis, and inflammatory bowel disease (IBD), which are collectively known as autoimmune diseases. Thus, autoimmune diseases are a constant and challenging burden for the healthcare system and society with an incidence of one in ten individuals. IBDs including Crohn's disease (CD) and ulcerative colitis (UC) are found worldwide and highly prevalent (>0.3%) in developed countries and affect about 6.8 million individuals globally. IBDs are characterized by aberrant inflammatory responses in the intestine, destruction of the epithelial barrier due to environmental factors, dysbiosis, genetic predisposition, diet, and lifestyle. The precise etiology of IBD remains unknown however, experimental studies suggest that dysregulation in the mucosal immune system is involved in the pathogenesis IBD. IBD is normally associated with the infiltration of immune cells, including macrophages, neutrophils, Th17, and type 1 T helper (Th1) cells into the colon, which secrete the proinflammatory cytokines interleukin 6 (IL-6), tumor necrosis factor-alpha (TNF-α) and interferon-gamma (IFN-γ) to induce chronic inflammation. The existing therapeutic approaches lessen the severity, weaken the immune system, and generate undesirable side effects even as the disease becomes refractory in some patients. In the modern world, it has been shown that roughly 50% of IBD patients are treated with alternative medications like probiotics, prebiotics, and medical cannabis. This justifies the necessity and continued search for safe, cost-effective, and new therapeutic approaches to restore immune homeostasis.

Cytoskeletal proteins, mainly actin and tubulin, are responsible for maintaining cellular morphology and function. Tubulin or microtubules (MTs) are highly dynamic and found in the cytoplasm and regulate important cellular functions like cell division, motility, cell shape changes, internal cellular transport, and organization. MTs dysfunction also has been implicated as a mediator of inflammation in multiple diseases and induces hyper-stimulatory response. It has been shown that MTs associated with transcription factor NF-κB stimulate apoptosis in response to suppression of MTs dynamics in MCF-7 cells. Further, the cytoskeleton also acts as a central regulator of innate immune cells as well as plays a role in immune synapse and polarization during T cell activation. Tubulin glycyases are required for primary cilia to control cell proliferation and colon development. Further, oxidant-induced cytoskeletal disruption is required for tissue injury, mucosal disruption, and IBD flare-up.

Studies from several IBD experimental models suggest that dysregulation in T cells, macrophages, and natural killer T (NKT) cells plays a role in the progression of IBD. Th17 is a helper T cell that secretes IL-17 and IL-22 and plays a crucial role in the progression of IBD. It has been shown that infiltrating Th17 cells increased in CD patients, and further Th17 cells increased in the dextran sodium sulfate (DSS) induced colitis. Much remains to be discovered related to the role of IL-17, as it appears to be important in both inducing and abating chemically induced colitis in mice.

Macrophages are a prominent cell type in active IBD and play an important role in the pathogenesis of Th1-mediated IBD. The heterogeneous precursors of macrophages, granulocytes, and dendritic cells during earlier stages of differentiation are identified as myeloid-derived suppressor cells (MDSCs). MDSCs show their ability to differentiate under the influence of selected cytokines and play a key role in the recruitment of other immune cells. MDSCs serve as potent immune response suppressors and expand during chronic inflammatory pathologies to serve as crucial players in the prevention of diseases. MDSCs frequency dramatically increased during intestinal inflammation in mice and these cells suppressed T-cell activation. Furthermore, MDSC frequency increases in the peripheral blood of patients with active colitis, possibly halting the development of more severe and possibly fatal colitis. Together, these studies identify MDSCs as a previously unexplored immune regulatory mechanism in IBD and alter the frequency of macrophages and other inflammatory cells in DSS-induced colitis.

Macrophages are the key inflammatory mediators in several autoimmune diseases including IBD. LPS-treated macrophage has long been studied as an in vitro model of inflammation and is generally used to test the anti-inflammatory properties of drugs. Further, macrophages are shown to rearrange the actin cytoskeleton to generate more filopodia and lamellopodia for migration toward the inflammatory site as well as for phagocytosis in their activated state. At the cellular level, Th17 cells have been gaining attention in the context of IBD.

Cellular morphology and function are generally maintained by the cytoskeletal proteins actin and tubulin. These cytoskeletal proteins also regulate immune cell functions, including cell migration, cell-cell interactions, phagocytosis, secretion, and antigen presentation. In tissue injury after colitis, mucosal disruption and symptomatic flare-ups are associated with oxidant-induced disruption of the cytoskeleton proteins. Furthermore, the progression of colitis results in part from the dysregulation of macrophages, Th17 helper T cells, and regulatory T cells (Tregs) that secrete cytokines interleukin 17 (IL-17), IL-10, and IL-22. IL-17 is important for both inducing and abating chemically induced colitis in mice, with an increase in the number of infiltrating Th17 cells in the colon of DSS-induced colitis in mice and in CD patients relative to healthy donors. Tregs play a crucial role in the control of intestinal inflammation and are required for the effective suppression of inflammation and experimental colitis. To this end, naturally arising Tregs have been shown to prevent or even cure the adoptive T cell transfer model of colitis. Further, mutations of Foxp3 in mice lead to uncontrolled T cell proliferation and increased production of Th1/Th2 and Th17 cytokines, suggesting a critical role for Tregs in immune homeostasis.

In both human IBD and experimental colitis, disruption of tight junction proteins reduces the integrity of the epithelial barrier, leading to an increase in the severity of colitis. The differential expression of tight junction proteins like occludin may lead to barrier dysfunction, mediate permeability, communicate between microbiota to the gut immune system, and induce symptoms of IBD. In DSS-induced colitis, DSS decreases the expression of occludin in colon epithelial cells and reduces occludin expression in models of intestinal inflammatory diseases, supporting its critical role in the maintenance of barrier integrity. Inflammatory cytokines such as interferon-gamma (IFN-γ) and tumor necrosis factor-alpha (TNF-α) have a differential effect on tight junctions, which may induce their permeability in IBD.

In the context of the lymphoid cell population, regulatory T cells and Th17 cells play crucial roles in immune diseases including IBD. CD4-positive T cells are enriched in the lesional tissue of IBD patients and play a prominent role in the progression of IBD. Under pathological conditions, the numbers of Th17 cells increase and trigger inflammatory reactions and IBD. An altered balance between Treg cells and T effector cells in the intestinal environment also contributes to IBD. Restoring the balance of Treg/Th17 cells effectively reduces inflammation and experimental colitis and which has been shown to have a reduction in the numbers of activated CD4⁺ T cells and Th17 cells is an indication of colitis abrogation. This is consistent with the ability of Tregs to cure IBD in mouse models, highlighting the induction of endogenous Tregs as a means of ameliorating disease.

Further, NF-κB-induced cytokines contribute to the stimulation, activation, and differentiation of immune cells, thus perpetuating colitis. Many established drugs mediate, at least in part, the anti-inflammatory effects via inhibition of NF-κB activity. NF-κB p65 is critically important in mediating many chronic inflammatory diseases including colitis. Inhibition of NF-κB activation has been suggested as an anti-inflammatory strategy. The compromised expression of the tight junction protein occludin, activation of transcription factor NF-κB and increased p-STAT3-mediated expression of cytokines and chemokines are key factors in the pathogenesis of IBD.

Inflammation is a defense mechanism of the immune system, but prolonged inflammation initiates diseases like inflammatory bowel disease (IBD). Inflammation is treated with analgesics, steroids, or nonsteroidal anti-inflammatory (NSAID) agents. The available conventional therapies for IBD have a high incidence of relapse and are associated with adverse side effects like osteoporosis, myopathy, hypertension, weight loss, stomach upset, and increased risk of infection. Thus, there is an urgent need for safe, novel anti-inflammatory therapeutic options for inflammatory mediated diseases, such as IBD.

### SUMMARY

The present invention discloses a nonsteroidal thiazole-based compound, DJ-X-013, including its synthesis and testing its effectiveness in ameliorating inflammation in a well-established model of inflammation, the DSS-induced model of colitis. DJ-X-013 displayed excellent physical and drug-likeness properties especially high GI absorption and no penetration on BBB with high bioavailability. Administration of DJ-X-013 reversed the severity of DSS-induced colitis and reduced the LPS-induced RAW264.7 macrophage inflammatory response. DJ-X-013 treatment reversed weight loss, improved colon length, and decreased disease severity. The increased frequency of pro-inflammatory markers including activated T cells, neutrophils, Th17 cells, and NF-κB levels was reduced by DJ-X-013 treatment. Further, DJ-X-013 induces MDSCs and differentially modulates NK, NKT, DCs, and cytoskeletal proteins to alter the severity of colitis. Taken together, these results indicate that DJ-X-013 abrogates colitis by inducing MDSCs, suppressing the Th17 cells, and modulating NF-κB and cytoskeletal proteins, thereby reducing inflammatory response and colitis. DJ-X-013 has potent anti-inflammatory functional properties brought about through four potentially parallel effects; i) inducing MDSCs frequency in the colon; ii) attenuating expression of inflammatory markers like TNF-α, IL-1B, and NF-κB; iii) reducing the local population of Th17, neutrophils and activated T cells; and iv) mitigating the migration of inflammatory cells to the colon LP through modulating cytoskeleton proteins.

The present invention also describes the design of a safe, nonsteroidal thiazole-based compound, DJ-X-025, that is predicted to share the anti-inflammatory activity observed in other thiazole-based compounds. DJ-X-025 displays potent anti-inflammatory properties that i) attenuate the expression of inflammatory markers in both LPS-induced RAW macrophage and in the colon of mice with DSS-induced experimental colitis; ii) differentially modulate numbers of Th17, Treg, and myeloid cells in the colon of these mice; and iii) reduce expression of NF-κB/p-STAT-3 and induce occludin in the colon of these mice which effectively ameliorates colitis. DJ-X-025, shown herein as Formula II, can reduce LPS-induced inflammation and differentially modulate the frequency of Th17, macrophage, dendritic cells (DCs), and Tregs in the colon of DSS-induced colitis. Evidence is also provided that administration of DJ-X-025 alters the localization of the cytoskeletal protein actin and tubulin and induces the expression of occludin, reduces the expression of phosphorylated signal transducer and activator of transcription (p-STAT3) and nuclear factor kappa-light-chain-enhancer of activated B cells (NF-κB) in the colon, and altogether reduces the levels of inflammatory cytokines and chemokines for effective suppression colitis. Thus, DJ-X-025 is presented as a novel therapeutic drug for the treatment of colitis and other inflammatory diseases.

This Summary is neither intended nor should it be construed as being representative of the full extent and scope of the present disclosure. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this Summary as well as in the attached drawings and the Description of Embodiments and no limitation as to the scope of the present disclosure is intended by either the inclusion or non-inclusion of elements, components, etc. in this Summary. Additional aspects of the present disclosure will become more readily apparent from the Description of Embodiments, particularly when taken together with the drawings.

### BRIEF DESCRIPTION OF FIGURES

FIGS. 1A-1F show the formulation and synthesis of DJ-X-013 and effects of DJ-X-013 on cell viability and morphology of RAW 264.7 cells under in vitro LPS stimulation. FIG. 1A shows a BOILED-Egg model for prediction of passive gastrointestinal (GI) adsorption and brain penetration for DJ-X-013, where the round, yellow “yolk” represents the BBB permeation region, the white oval represents the human intestinal absorption (HIA) region, and the gray rectangle represents the low absorption and limited brain permeation region. FIG. 1B shows the graphical output of the ADME prediction calculated for DJ-X-013 by SwissADME.³⁹ The pink shaded area represents the physicochemical space that is suitable for oral bioavailability, while DJ-X-013 is shown as a red line, calculated from its physical properties, which were assessed based on its predicted lipophilicity (LIPO), size (SIZE), polarity (POLAR), insolubility (INSOLU), insaturation (INSATU), and flexibility (FLEX). FIG. 1C shows the synthesis of DJ-X-013 using two methods. method A (top panel) from 5-bromo-1,2,3-trimethoxybenzene (compound 1) and 4-phenylthiazole-2-carbaldehyde (compound 2) and method B (bottom panel) from 3,4,5-trimethoxyphenylmagnesiumbromide (compound 4) and compound 2. FIG. 1D shows cell viability by MTT assay after in vitro LPS stimulation in the absence or presence of DJ-X-013 treatment. Statistical analysis was performed using one-way ANOVA followed by Dunnett's post hoc test; n=3 per group. FIG. 1E shows cell morphology after treatment with LPS or LPS plus 10 μM or 50 μM DJ-X-013 was determined by phase contrast microscopy (scale bar 100 μm). Elongated cells visible after treatment with LPS alone are indicated by red arrows. FIG. 1F shows a morphometric analysis of untreated cells and cells treated with LPS alone or LPS+DJ-X-013 (10 or 50 μM) was performed on phase contrast images using ImageJ software (NIH). The parameters examined were area (μm²), perimeter (μm), major axis (μm), minor axis (μm), aspect ratio (arbitrary units), and circularity (arbitrary units). Statistical analysis was performed using one-way ANOVA followed by Tukey's multiple post hoc tests; n=75 for each group and/or cell type. LPS treatment alone resulted in a round—LPS (R) or elongated—LPS (E) cell morphology, while control cells and those in the presence of 10 or 50 μM DJ-X-013 were mostly round, suggesting that DJ-X-013 treatment either induces elongated cells to revert to round cells or dampens processes involved in elongation. Data are presented as mean values±SEM, ns p>0.05; *p<0.05; **p<0.01; ***p<0.001; ****p<0.0001.

FIGS. 2A-2C show that DJ-X-013 attenuates in vitro LPS-stimulated RAW 264.7 cell migration through cytoskeletal protein expression regulation. FIG. 2A shows representative phase contrast images show the closure of a scratch wound in a monolayer of LPS-stimulated RAW 264.7 macrophages in the presence or absence of DJ-X-013 over time, following incubation of 0, 4, 8, 24, and 28 h. Scale bar 400 μm. FIG. 2B is a plot showing that DJ-X-013 reduces cell migration rate. Statistical analysis was performed using one-way ANOVA followed by Dunnett's post hoc test; n=3. Data are presented as mean values±SEM, ns p>0.05; ***p<0.001; ****p<0.0001. FIG. 1C shows immunofluorescence confocal microscopic images depict the alteration of actin and tubulin after treatment of LPS-stimulated RAW 264.7 macrophages with 10 or 50 μM DJ-X-013. Scale bar 20 μm; detected proteins (color): F-actin (red), tubulin (green), and DAPI for nucleus (blue). These results suggest that DJ-X-013 impedes the migration of macrophages and might modulate the polymerization or depolymerization of cytoskeletal proteins.

FIGS. 3A-3E show that DJ-X-013 treatment diminishes inflammatory markers in LPS-stimulated RAW 264.7 cells in vitro. FIG. 3A shows that in LPS-stimulated RAW 264.7 cells treated in the absence or presence of 10 or 50 μM DJ-X-013, the expression of the mRNAs encoding pro-inflammatory markers TNF-α, iNOS, IL-6, IL-1B, STAT3, and NF-κB was analyzed using reverse transcription-quantitative polymerase chain reaction (RT-qPCR). Whereas LPS alone stimulated the expression of these inflammatory genes, it was inhibited by DX-J-013. FIG. 3B shows that after a similar treatment of RAW 264.7 cells, flow cytometry was used to examine the expression of TNF-α and iNOS proteins, lower right quadrants of top and bottom rows show the percentage of TNF-α+ and iNOS+ cells, respectively. Representative images from one of three experiments that produced similar results are shown. This experiment shows that DJ-X-013 treatment reduced TNF-α+ and iNOS+ expression in LPS-stimulated RAW 264.7 cells. FIG. 3C shows a multiplex assay analysis of CREB, ERK, JNK, STAT3, STAT5, and NF-κB signaling after similar treatment. The unit mean fluorescence intensity (MFI) was directly proportional to the concentration. FIG. 3D shows that after similar treatment, cells were lysed, and the accumulation of NF-κB was analyzed by immunoblot. FIG. 3E shows relative NF-κB expression after LPS and DJ-X-013 treatment. In totality, DJ-X-013 reduced the expression of mRNAs and proteins encoded by inflammation-associated genes. Statistical analysis was performed by one-way ANOVA followed by Dunnett's post hoc test in FIG. 3A n=3, FIG. 3C n=4-5, and FIG. 3D n=3. Data are presented as mean±SEM., ns p>0.05, *p<0.05, **p<0.01, ***p<0.001, ****p<0.0001.

FIGS. 4A-4H show DJ-X-013 treatment ameliorates DSS-induced mouse colitis. Change in body weight and colon length after DJ-X-013 treatment in DSS-induced colitis. C57BL/6 mice received daily treatment in drinking water for 7 days as follows: control mice received plain water+vehicle (●), DSS-treated mice received 3.5% DSS+vehicle (▪), and DSS+DJ-X-013 mice received 3.5% DSS+20 mg/kg body weight DJ-X-013 (▴). FIG. 4A shows that the body weight of each mouse was recorded every day, and the change from the initial body weight was expressed as a percentage change in body weight (n=5). FIG. 4B shows the macroscopic view of colon length and FIG. 4C shows the related plot. FIG. 4D shows the macroscopic view of spleen size and FIG. 4E shows the related plot of spleen weight. FIG. 4F shows MLNs were isolated from each mouse and MLN cells were isolated and counted; shown is the count per mouse. FIG. 4G shows Histological analysis of H&E-stained colon tissue from each experimental group is shown in representative microphotographs. Scale bars 200 μm in upper panels and 50 μm in lower panels, respectively. The epithelial fold length and sub-mucosal width or thickness were measured from histological images using ImageJ software (NIH) and the inflammation score was determined based on epithelial damage, loss of goblet cells, crypt disruption, and infiltration of inflammatory cells, as shown in FIG. 4H. One-way ANOVA followed by Dunnett's post hoc test was applied in FIG. 4B n=5, FIG. 4D n=6, FIG. 4F n=5-8, and FIG. 4H n=65 for epithelial fold length, n=105 for submucosal thickness and n=16 for inflammation score. Data are presented as mean±SEM., ns p>0.05, *p<0.05, **p<0.01, ****p<0.0001.

FIG. 5A and FIG. 5B show that DJ-X-013 resolves DSS-induced colitis by inhibiting neutrophils, TNF-α producing macrophages, and activated monocytes. Spleens, MLNs, and colon LP immune cells were isolated from the three groups of mice (control, DSS, DSS+DJ-X-013) on day 8 and stained with antibodies specific for CD11b⁺, Ly6C, and TNF-α. FIG. 5A depicts percentages of CD11b⁺ macrophages producing TNF-α (left column) and of activated monocytes (CD11b⁺Ly6C⁺; right column) are shown in the upper right quadrants. FIG. 5B shows the percentages of neutrophils detected in the indicated organ are shown (Ly6C⁺ cells are boxed). Representative data from one of the three experiments that produced similar results are shown.

FIG. 6A and FIG. 6B show that treatment of mice with DSS-induced colitis improves colitis symptoms by differentially modulating T cells and natural killer cells. Spleens, MLNs, and colon LP immune cells were isolated from the three groups of mice (control, DSS, DSS+DJ-X-013) on day 8 and stained with antibodies specific for CD3, CD4, CXCR3, and NK1.1. FIG. 6A illustrates the percentages of activated T cells (CD4⁺CXCR3⁺ cells in the upper right quadrants). FIG. 6B describes the percentages of NK cells (CD3⁻NK1.1; upper left quadrants) and NKT cells (CD3⁺NK1.1; upper right quadrants). Representative data from one of the three experiments that produced similar results are shown.

FIG. 7A and FIG. 7B show that DJ-X-013 prevents colitis mediating through MDSCs and DCs cells. Spleens, MLNs, and colon LP immune cells were isolated from the three groups of mice (control, DSS, DSS+DJ-X-013) on day 8 and stained with antibodies specific for CD11b, GR-1, and CD11c. FIG. 7A shows the percentages of MDSCs (CD11b⁺GR-1⁺ are shown upper right quadrants). FIG. 7B displays the percentages of dendritic cells (CD11b⁺ and CD11c⁺; upper right quadrants). Representative data from one of the three experiments that produced similar results are shown.

FIGS. 8A-8E show that DJ-X-013 mitigates colitis by attenuating Th17 cells and inflammatory markers. FIG. 8A shows that spleens, MLNs, and colon LP immune cells were isolated from the three groups of mice (control, DSS, DSS+DJ-X-013) on day 8 and stained for CD4 and IL-17A. The percentages of Th17 cells gated on CD4⁺ cells (CD4⁺IL-17A⁺) are shown in the upper right quadrants. Representative data from one of the three experiments that produced similar results are shown. FIG. 8B depicts Th17 cell numbers in the spleen, MLNs, and colon LP. FIG. 8C shows the inflammatory TNF-α, iNOS, CXCR3, IL-1B, IL-17F, and IFN-γ gene expression in colon tissue using RT-qPCR. FIG. 8D shows representative immunoblots and FIG. 8E shows their relative quantification of NF-κB (p65) in colon tissue. FIG. 8F shows representative immunoblots and FIG. 8G shows their relative quantification non-canonical NF-kB (p100/52) in the colon tissue. One-way ANOVA followed by Dunnett's post hoc test was applied in FIG. 8B n=5 and FIGS. 8A-8C n=3. Data are presented as mean±SEM., ns p>0.05, *p<0.05, **p<0.01, ***p<0.001, ****p<0.0001.

FIG. 9 shows body weight restoration of mice treated with DSS (3.5%) in the absence and presence of 20 mg/kg, 40 mg/kg, or 80 mg/kg DJ-X-013 (n=3).

FIG. 10 shows a Multiplex assay analysis of p38, p70S6K and, Akt signaling after DSS and DJ-X-013 treatment in RAW264.7 macrophage cells (n=3-5).

FIG. 11 shows cytokine and chemokine levels in serum of control, DSS, and DJ-X-013 treated colitis of mouse (n=3-5).

FIG. 12 is a graphical abstract showing the differences between colitis and colitis treated with DJ-X-13.

FIGS. 13A, 13B, 13C, 13D and 13E depict the formulation and synthesis of DJ-X-025 and cell viability of cultured RAW 264.7 macrophages treated with LPS and DJ-X-025. FIG. 13A shows the structure and basic components of DJ-X-025. FIG. 13B shows a BOILED-Egg model predicting the BBB permeation (yellow sphere) and human intestinal absorption (white oval) properties of a drug; the gray area surrounding the egg is the low absorption and limited brain permeation region. The physical properties of DJ-X-025 (red hollow sphere) predict it will be P-glycoprotein positive, have no BBB penetration, and show high intestinal absorption. FIG. 13C shows a graphical output of the ADME studies for DJ-X-025 calculated using SwissADME. FIG. 13D shows the synthesis of DJ-X-025. Reagents and conditions: (a) THF, RT; (b) Dess-Martin Periodinane, DCM, RT; (c) BBr₃, DCM, 0° C. to RT. FIG. 13E shows an estimation of cellular apoptosis of LPS stimulated RAW 264.7 macrophages treated with 5, 10, 20, and 50 μM concentrations of DJ-X-025, using FITC Annexin V apoptosis detection assay (n=3 in triplicate).

FIGS. 14A, 14B, 14C, 14D, 14E, 14F and 14G depict that DJ-X-025 induced the morphological alteration of LPS-stimulated RAW 264.7 cells. FIG. 14A shows Representative images of phase contrast microscopy (scale bar: 200 μm) show the phenotypic changes under different conditions. The morphological appearance of round (RO, white) and elongated (EL, yellow) cells are indicated using arrows. Morphological alterations were validated through morphometric analysis of phase contrast images using ImageJ software (NIH). FIG. 14B shows the morphological features changes to area (μm²). FIG. 14C shows the morphological features changes to area perimeter (μm). FIG. 14D shows the morphological features changes to the major axis (μm). FIG. 14E shows the morphological features changes to the minor axis (μm). FIG. 14F shows the morphological features changes to the aspect ratio (arbitrary units). FIG. 14G shows the morphological features changes to circularity (arbitrary units). Statistical analysis was performed using one-way ANOVA followed by Tukey's multiple comparison tests, n=125 for each cell type and/or group. Data are presented as mean values±SEM; *p<0.05, **p<0.01, ***p<0.001, ****p<0.0001.

FIGS. 15A, 15B, 15C and 15D depict that DJ-X-025 modulated the distribution of cytoskeletal protein actin and tubulin of LPS-activated RAW 264.7 cells. FIG. 15A shows representative fluorescence confocal microscopy images stained with antibodies specific for actin (red) show the localization of actin at the intercellular junction of the round cell periphery (white arrows). FIG. 15B shows representative fluorescence confocal microscopy images stained with antibodies specific for tubulin (green), white arrows illustrate the higher tubulin density at the intercellular junction of the round cell periphery while yellow arrows denote less intense tubulin in the cytoplasm of elongated cells. FIG. 15C shows representative fluorescence confocal microscopy images show the DAPI-stained nucleus (blue). FIG. 15D shows representative merged fluorescence confocal microscopy images show the colocalization of actin, tubulin, and the nucleus together. Scale bars for all images are 50 μm.

FIGS. 16A, 16B and 16C depict that DJ-X-025 attenuated the expression of inflammatory markers of LPS-treated RAW 264.7 cells. FIG. 16A shows results after LPS challenge of RT-qPCR analysis to monitor the expression of IL-1B, IL-6, IL-10, TNF-α, iNOS, NF-κB, SIRT1, STAT3, and STAT5 mRNA. FIG. 16B shows that for flow cytometric analysis, cells were initially gated on side scatter (SSC) vs. forward scatter (FSC) for TNF-α, and iNOS. Flow cytometric dot plot (lower right quadrants: TNF-α⁺ cells) and FIG. 16C shows a graph showing the percentage of TNF-α positive cells. FIG. 16D shows that for flow cytometric analysis, cells were initially gated on side scatter (SSC) vs. forward scatter (FSC) for TNF-α, and iNOS. Flow cytometric dot plot (lower right quadrants: iNOS+ cells) and FIG. 16E shows a graph showing the percentage of iNOS-positive cells. Statistical analysis was performed using one-way ANOVA followed by Dunnett's post hoc test (n=3 in triplicate). Data are presented as mean values±SEM; *p<0.05, ***p<0.001, ****p<0.0001.

FIGS. 17A, 17B, 17C, 17D, 17E, 17F and 17G depict that DJ-X-025 treatment alleviated DSS-induced colitis in mice. C57BL/6 mice were treated as follows for 7 days: control mice were given normal drinking water and treated with vehicle by gavage, mice in the DSS group were given drinking water containing 3.5% DSS and treated with vehicle by gavage, and mice in the DSS+DJ-X-025 group were given drinking water containing 3.5% DSS and treated with 20 mg/kg body weight DJ-X-025 by gavage. At the experimental endpoint on day 8, the mice were sacrificed. FIG. 17A shows the body weight of each mouse was recorded every day and the alteration of body weight from the initial body weight was represented as a percentage change in body weight (n=6). FIG. 17B shows a representative macroscopic view of colon length in a mouse from each group after sacrifice on day 8 and related graph (n=6). FIG. 17D shows a representative macroscopic view of mouse spleen size after sacrifice on day 8 and FIG. 17C shows a graph of spleen weight (n=6) and average splenocyte number from three experimental repeats (n=9). FIG. 17E shows the average mesenteric lymph node cell number from three experimental repeats (n=9). FIG. 17F shows a representative bright field histological microphotographs of H&E-stained colon tissue sections from control, DSS, and DSS+DJ-X-025 groups. *MM indicates the muscularis mucosae smooth muscle layer. Scale bars represent 200 μm in the upper panels and 50 μm in the lower panels FIG. 17G shows the thickness of the muscularis mucosae was measured from twenty representative histological images using ImageJ software (n=200 measurement using twenty images from each experimental group). FIG. 17H shows a graphical representation of an inflammation score determined based on epithelial damage, loss of goblet cells, disruption of crypt structure, and infiltration of inflammatory cells (n=20 fields were selected from each study group of three experimental repeats). Statistical analysis was performed using one-way ANOVA followed by Dunnett's post hoc test. Data are presented as mean values±SEM; **p<0.01, ***p<0.001, ****p<0.0001.

FIGS. 18A, 18B, 18C, 18D, 18E, 18F, 18G, 18H, 18I, 18J, 18K, 18L, 18M, 18N, 180 and 18P show that DJ-X-025 treatment diminished different myeloid cell populations of DSS-induced colitis in mice. Spleens, MLNs, and colon LP immune cells were isolated from mice in the control group, DSS group, and DSS+DJ-X-025 groups on day 8 and stained with antibodies specific for cell markers Ly6C (neutrophils), CD11b and CD11c (dendritic cells), CD11b and F4/80 (macrophages), and TNF-α. Cells were initially gated on side scatter (SSC) vs. forward scatter (FSC), then SSC vs Ly6C, or SSC vs CD11b, CD11b⁺ cells were further gated with an appropriate marker to obtain specific myeloid cells. FIGS. 18A and 18B show the percentages of neutrophils (Ly6C⁺) in the spleens (FIG. 18A) and the MLNs (FIG. 18B). FIGS. 18C and 18D show the percentages of neutrophils (Ly6C⁺) in the LP. FIG. 18C shows one representative set of density plots for each group (inside box: Ly6C⁺ cells) and the graph in FIG. 18D compares their relative proportions. FIGS. 18E and 18F show the percentages of dendritic cells (DCs; CD11b⁺CD11c⁺) in the spleens (FIG. 6D0 and the MLNs (FIG. 18F). FIGS. 18G and 18H show the percentages of DCs (CD11b⁺CD11c⁺) in the LP. FIG. 18G shows a representative set of density plots for each group (inside box: CD11b⁺CD11c⁺ cells) and the graph in FIG. 18H compares their relative proportions. FIGS. 18I and 18J show the percentages of macrophages (CD11b⁺F4/80) in the spleen (FIG. 18I) and the MLN (FIG. 6J). FIGS. 18K and 18L show the percentages of macrophages (CD11b⁺F4/80) in the LP. FIG. 18K shows one representative set of density plots for each group (inside box: CD11b⁺F4/80 cells) and the graph in FIG. 18L compares their relative proportions. FIGS. 18M and 18N show the percentages of TNF-α producing cells (CD11b⁺TNF-α) in the spleens (FIG. 18M) and the MLN (FIG. 18N). FIGS. 18O and 18P show the percentages of TNF-α producing cells (CD11b⁺TNF-α) in the LP. FIG. 18O shows a representative set of density plots for each group (upper right quadrants: CD11b⁺ TNF-α cells) and the graph in FIG. 18P compares their relative proportions. For FIGS. 18A-18H, a total of n=9 (presented as the average of 3 experiments), while for FIGS. 181-18P, n=6.

FIGS. 19A, 19B, 19C, 19D, 19E, 19F, 19G, 19H, 19I, 19J, 19K and 19L show that DJ-X-025 treatment modulated various T cell populations in mice with DSS-induced colitis. Immune cells from spleens, MLNs, and colon LP were isolated from mice in the control groups, DSS groups, and DSS+DJ-X-025 groups on day 8 and stained with antibodies specific for CD4, CXCR3, IL-17, and Foxp3. Cells were initially gated on side scatter (SSC) vs. forward scatter (FSC), then SSC vs. CD4; CD4⁺ cells were further gated with appropriate markers to identify different populations of T cells. FIGS. 19A and 19B show the percentages of activated T cells (CD4⁺CXCR3) in the spleen (FIG. 19A) and the MLNs (FIG. 19B). FIGS. 19C and 19D show the percentages of activated T cells (CD4⁺CXCR3) in LP. FIG. 19C shows one representative set of density for each group (upper right quadrants: CD4⁺CXCR3⁺ cells) and the graph in FIG. 19D compares their relative proportions. FIGS. 19E and 19F show the percentages of Th17 cells (CD4⁺IL-17) in the spleen (FIG. 19E) and the MLNs (FIG. 19F). FIGS. 19G and 19H show the percentages of Th17 cells (CD4⁺IL-17) in the LP. FIG. 7G shows one representative set of density plots for each group (inside box: CD4⁺IL-17 cells) and the graph in FIG. 19H compares their relative proportions. FIGS. 19I and 19J show the percentages of Treg cells (CD4⁺Foxp3) in the spleen (FIG. 19I) and MLNs (FIG. 19J). FIGS. 19K and 19L show the percentages of Treg cells (CD4⁺Foxp3) in the LP. FIG. 19K shows one representative set of density plots for each group (inside box: CD4⁺IL-17 cells) and the graph in FIG. 19L compares their relative proportions. For FIGS. 19A-19H, a total of n=9 (presented as an average of 3 experiments), while for FIGS. 191-19L, n=6.

FIGS. 20A, 20B, 20C and 20D show that DJ-X-025 treatment reduced inflammation and reestablished epithelial markers in DSS-induced colitis in mice. FIG. 20A shows RNA was isolated from colon tissue collected on day 8 from mice in the control groups, DSS groups, DSS+DJ-X-025 groups, and the relative expression of the IL-1B, CXCR3, IL-17F, Foxp3, NF-κB, and TNF-α genes from three experimental repeats were examined using RT-qPCR. FIG. 20B shows that total protein was isolated from colon tissue collected on day 8 from mice in the control, DSS, and DSS+DJ-X-025 groups, and the relative expression of the p-STAT3, NF-κB, and occluding proteins (relative to housekeeping gene β-actin) from three experimental repeats was examined by immunoblot. Representative immunoblot images and densitometric analysis are shown in FIG. 20C. FIG. 20D shows that serum was prepared from whole blood collected from mice on day 8 in the control, DSS, and DSS+DJ-X-025 groups. Levels [picograms per milliliter (pg/ml)] of serum cytokines and chemokines (viz. CXCL1, CXCL10, CCL5, MCP-1, MCP-3, CCL4, IL-23, LIF, Il-6, IL-17A, TNF-α, and IFN-γ) were compared using a multiplex assay. Statistical analysis was performed using one-way ANOVA followed by Dunnett's post hoc test (n=9). Data are presented as mean values±SEM; *p<0.05, **p<0.01, ***p<0.001.

FIG. 21 depicts the design and strategy of DJ-X-025 combined with gallic acid.

FIGS. 22A, 22B and 22C show that DJ-X-025 altered MC38 cell morphology and RNA transcripts. FIG. 22A shows representative phase contrast microscopic images (100× magnification, scale bar: 400 μm) of MC38 cells showing the morphological changes after control, 10 μM, and 20 μM DJ-X-025 treatment in a large field of view. FIG. 22B shows representative phase contrast microscopic images (200× magnification, scale bar: 200 μm) of MC38 cells showing the morphological changes after control, 10 μM, and 20 μM DJ-X-025 treatment in prominent single cell detail view. The morphological appearance of elongated and round cells are indicated using yellow and blue arrows respectively in FIGS. 22A and 22B. FIG. 22C shows that RT-qPCR analysis demonstrates expression of NF-κB, STAT3, IL-6, and iNOS mRNA level (n=3, biological replicates).

FIGS. 23A and 23B show that DJ-X-025 slightly decreased the expression of IL-6 of LPS-treated RAW 264.7 cells. FIG. 23A shows a flow cytometric contour plot (inner box R5: IL-6⁺ cells). FIG. 23B is a graph showing the percentage of IL-6 positive cells. Statistical analysis was performed using one-way ANOVA followed by Dunnett's post hoc test (n=3, biological replicates). Data are presented as mean values±SEM; ****p<0.0001.

FIG. 24 shows that DJ-X-025 alters the level of serum cytokine and chemokines in DSS-induced colitis. Serum was collected from mice on day 8 in the control, DSS, and DSS+DJ-X-025 groups. Levels [picograms per milliliter (pg/ml)] of serum cytokines and chemokines (viz. IL-1B, IFN-α, M-CSF, G-CSF, GM-CSF, CCL3, CXCL-5, and CXCL2) were compared using a multiplex assay. Statistical analysis was performed using one-way ANOVA followed by Dunnett's post hoc test (n=3). Data are presented as mean values±SEM; *p<0.05, **p<0.01, ***p<0.001, ****p<0.0001.

### DETAILED DESCRIPTION

This disclosure provides a nonsteroidal, thiazole-based compound with anti-inflammatory properties.

In some embodiments, the presently disclosed subject matter provides a compound having the structure of Formula (I):

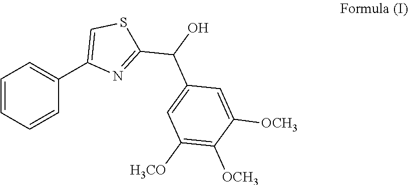 [SMILES: COc1cc(C(O)c2nc(-c3ccccc3)cs2)cc(OC)c1OC]

a prodrug thereof, or a pharmaceutically acceptable salts thereof.

In certain embodiments, the compound of Formula (I) is formulated as part of a pharmaceutical composition, further comprising at least one pharmaceutically acceptable carrier. In certain embodiments, the pharmaceutical composition further comprises at least one additional therapeutic agent. In other embodiments, the at least one additional therapeutic agent is at least one steroidal agent.

In some embodiments, the presently disclosed subject matter provides a compound having the structure of Formula (II):

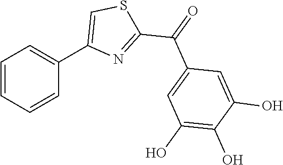 [SMILES: O=C(c1cc(O)c(O)c(O)c1)c1nc(-c2ccccc2)cs1]

a prodrug thereof, or a pharmaceutically acceptable salts thereof.

In certain embodiments, the compound of Formula (II) is formulated as part of a pharmaceutical composition, further comprising at least one pharmaceutically acceptable carrier. In certain embodiments, the pharmaceutical composition further comprises at least one additional therapeutic agent. In other embodiments, the at least one additional therapeutic agent is at least one steroidal agent.

Another aspect of this disclosure provides pharmaceutical kits containing a pharmaceutical composition of this disclosure, prescribing information for the composition, and a container.

Another aspect of this disclosure provides methods for treating an inflammatory disease in a subject, including administering to the subject a therapeutically effective amount of the compound of Formula (I) or Formula (II), or a pharmaceutically acceptable salt thereof.

A further aspect of the invention provides for methods of treating chronic inflammatory diseases by administering an effective amount of the compound of Formula (I) or Formula (II), or a prodrug or pharmaceutically acceptable salt thereof, to a patient in need of such treatment.

A further aspect of the invention provides a method of treating an inflammatory disease in a subject, including administering to the subject a therapeutically effective amount of the compound of Formula (I) or Formula (II), or a pharmaceutically acceptable salt thereof, wherein the inflammatory disease is rheumatoid arthritis, multiple sclerosis, systemic lupus erythematosus, type 1 diabetes, psoriasis, or inflammatory bowel disease.

A further aspect of the invention provides a method of treating an inflammatory disease in a subject, including administering to the subject a therapeutically effective amount of the compound of Formula (I) or Formula (II), or a pharmaceutically acceptable salt thereof, wherein the inflammatory disease is Crohn's disease or ulcerative colitis.

The invention disclosed herein encompasses various embodiments wherein the subject of treatment may be a mammal, including humans. The therapeutic compounds and methods described herein are applicable to both non-human mammals and humans, with the aim of providing effective treatment for a wide range of medical conditions.

For non-human mammalian embodiments, the therapeutic compounds and methods may be employed in veterinary medicine for the treatment of animals such as dogs, cats, horses, cows, pigs, rodents, and other domesticated or wild mammals. These embodiments may involve administering the therapeutic compounds via suitable routes and dosage regimens tailored to the species, size, and health condition of the animal.

For human embodiments, the therapeutic compounds and methods are intended for use in the treatment of various medical conditions in humans. These conditions may include, but are not limited to, inflammatory diseases.

Compounds described herein are synthesized using any suitable procedures starting from compounds that are available from commercial sources or are prepared using procedures described herein. General methods for the preparation of compounds as described herein are modified by the use of appropriate reagents and conditions, for the introduction of the various moieties found in the Formulas as provided herein.

In the context of the present invention, the term “patient” or “subject” refers to an individual, whether human or non-human mammal, who is receiving or undergoing treatment with the therapeutic compounds or methods disclosed herein. In human embodiments, the patient may be an individual diagnosed with a medical condition or disease for which treatment is indicated. In non-human mammalian embodiments, the subject may be an animal, such as a dog, cat, horse, rodent, or other mammalian species, for which therapeutic intervention is necessary or desirable. The terms “patient” or “subject” are used interchangeably throughout this specification to refer to the recipient of treatment, regardless of whether they are human or non-human mammals.

“Administration of” or “administering” refers to the act of delivering or applying the therapeutic compound to a patient or subject for the purpose of treating a medical condition or disease. Administration may be performed by healthcare professionals, caregivers, or the patients themselves, whether solely or under the guidance and supervision of qualified personnel.

Treating or treatment refers to the administration of a therapeutic agent to a patient with the intention of alleviating, curing, or preventing a disease or medical condition. The term may refer to any mode of therapy, including pharmacological, surgical, radiation, or other types of therapy. The term “preventing” may also encompass prophylactic treatment, which is the administration of a therapeutic agent to prevent the onset or recurrence of a disease or medical condition.

A therapeutically effective amount refers to a quantity of the active ingredient(s) of the compound of this disclosure that exhibits the desired therapeutic effect in a patient. The amount may vary depending on various factors, including the disease or disorder to be treated, the patient's age and health condition, the route of administration, and the desired therapeutic effect. A therapeutically effective amount may be determined by one skilled in the art using routine experimentation, and may be expressed as a range or a specific value.

A “pharmaceutically acceptable salt” refers to a salt form of a compound that is acceptable for use in pharmaceutical formulations. These salts must be safe for administration to patients and provide the desired pharmacological activity when used in therapeutically effective amounts. Such salts are commonly formed to enhance solubility, stability, bioavailability, or ease of crystallization of the active therapeutic compound without altering its intrinsic therapeutic properties. Pharmaceutically acceptable salts may include those derived from pharmaceutically acceptable inorganic or organic acids and bases. The salt form is typically prepared by reacting the compound with an appropriate acid or base to form a salt. The choice of the acid or base may depend on various factors, including the solubility, stability, and bioavailability of the salt. A pharmaceutically acceptable salt should be non-toxic and should not cause any significant adverse effects in the patient. Examples of pharmaceutically acceptable salts include, but are not limited to, hydrochloride, sulfate, citrate, tartrate, maleate, fumarate, mesylate, besylate, lactate, acetate, benzoate, succinate, and phosphate salts.

A “prodrug” refers to a pharmacologically inactive compound that is designed to be converted into an active drug in the body. Prodrugs may be advantageous for various reasons, including improving the pharmacokinetic properties of a drug, enhancing its bioavailability, and reducing its toxicity. Prodrugs may be designed to be converted into the active drug through various mechanisms, including hydrolysis, oxidation, reduction, or enzymatic cleavage.

A “pharmaceutical composition” refers to a formulation that comprises one or more active ingredients and one or more pharmaceutically acceptable carriers. The pharmaceutical composition facilitates administration of the compound to a patient or subject. The composition may be in any suitable form, including but not limited to tablets, capsules, powders, solutions, suspensions, emulsions, gels, creams, ointments, patches, and injectable formulations. The choice of a pharmaceutical composition may depend on various factors, including the type and route of administration, the stability of the active ingredient, and the desired therapeutic effect. The composition may be prepared by any suitable method, including but not limited to blending, mixing, granulating, compressing, or lyophilizing.

A “pharmaceutically acceptable carrier” refers to a substance or a combination of substances that are inert, non-toxic, and compatible with the active ingredient(s) of the compound of the present disclosure. The carrier may be a solid, liquid, or a gas and may include, but is not limited to, excipients, diluents, binders, lubricants, disintegrants, fillers, and solvents. The choice of a pharmaceutically acceptable carrier may depend on various factors, including the type and route of administration, the stability of the active ingredient, and the desired therapeutic effect.

The pharmaceutical composition of the present invention can be administered through various routes to achieve therapeutic effects tailored achieve the desired therapeutic effect. These routes include oral, parenteral, rectal, topical, nasal, buccal, vaginal, and inhalation administration. Orally administered pharmaceutical compositions are ingested through the mouth and are typically in the form of tablets, capsules, solutions, or suspensions, offering convenient and widely accepted dosing options. Dosage forms and strengths can vary depending on factors such as patient age, severity of inflammation, and desired therapeutic outcome. Parenteral administration involves delivering the pharmaceutical composition directly into the body through means other than the digestive tract, such as subcutaneous (SC), intravenous (IV), intramuscular (IM), intraperitoneal (IP), or intrathecal routes. This route bypasses the gastrointestinal system, allowing for rapid absorption and systemic distribution of the therapeutic agent making it suitable for acute conditions or situations requiring immediate therapeutic intervention. Pharmaceutical compositions for intravenous administration are typically in the form of sterile solutions or suspensions. Rectal administration involves the insertion of suppositories, enemas or rectal gels into the rectum for localized or systemic effects. This route of administration is particularly useful for patients unable to take oral medications or requiring local treatment of conditions such as hemorrhoids or inflammatory bowel disease. Topical administration entails applying creams, ointments, gels, or patches directly onto the skin or mucous membranes for targeted localized treatment while minimizing systemic side effects. Nasal administration involves delivering sprays or drops into the nasal cavity for systemic or local effects. Buccal administration involves placing tablets or patches between the cheek and gum for absorption through the buccal mucosa. Vaginal administration involves the insertion of suppositories, creams, or tablets into the vagina for local or systemic effects. Inhalation administration involves inhaling aerosols, powders, or vapors into the respiratory tract for rapid absorption and distribution of the therapeutic agent. Each route of administration offers unique advantages in terms of efficacy, convenience, and patient compliance, allowing for tailored treatment approaches to optimize therapeutic outcomes.

Dosage and administration regimens may vary depending on factors such as the patient's age, weight, medical condition, and response to treatment. The dosage range may be from about 10 mg to about 1000 mg per day, depending on the disease or disorder to be treated, the patient's age and health condition, and the desired therapeutic effect. The appropriate dosage and administration schedule should be determined by a qualified healthcare professional based on individual patient characteristics and therapeutic goals.

### EXAMPLES

The following methods were used to conduct the experiments described in Examples 1-23, below:

Cell Culture with LPS and DJ-X-013 Treatment

Mouse macrophage RAW264.7 cells (Cat no. TIB-71, ATCC, USA) were cultured in Dulbecco's modified essential medium (DMEM) medium (Cat no 10-027-CV, Corning, NY) supplemented with 10% fetal bovine serum (FBS) and 1% antibiotic solution (penicillin/streptomycin) and maintained at 37° C., 5% CO₂ in an incubator as described previously.⁴⁰ Cells were seeded in appropriate dishes and 24 h, later were treated with 100 ng/mL LPS (Cat. no. L4391, Millipore Sigma, St. Louis, MO) and 10 μM or 50 μM DJ-X-013 for 24 h. Cells without any treatment of LPS and DJ-X-013 designated as control and treated with only LPS were considered as the LPS group. The groups analyzed were as follows: cells in the control group were incubated without LPS or DJ-X-013, cells in the LPS group were treated with only LPS, cells in the 10 μM group were treated with LPS+10 μM DJ-X-013, and cells in the 50 μM group were treated with LPS+50 μM of DJ-X-013.

### Cell Viability Assay

RAW264.7 cells were seeded in 96 well plates at 5×10³ cells per well and were incubated in complete DMEM medium at 37° C., 5% CO₂ for 20-24 h. Cells were then stimulated with LPS (100 ng/mL) and simultaneously treated with compound DJ-X-013 at 1, 5, 10, 20, 50, or 100 μM and incubated at 37° C. After 24 h, the medium was replaced by fresh medium. 3-[4,5-dimethylthiazol-2-yl]-2,5 diphenyl tetrazolium bromide (MTT) reagent (Cat. no. M6494, Invitrogen, Carlsbad, CA, USA) [dissolved in phosphate-buffered saline (PBS)] was added each well with final concentration of 0.5 mg/ml, and the plate was incubated in the dark at 37° C. for 4 h. The medium containing MTT was carefully removed, the deposited formazan crystals were completely dissolved in dimethyl sulfoxide (DMSO) by incubating the plate in the dark at room temperature (RT) for 15 min., in shaking condition and the absorbance of each well was measured at a wavelength of 570 nm in a plate reader (CYTATION 5 imaging reader, Agilent BioTek, Santa Clara, CA). In this assay, the blank wells received the same treatment but without cells, while cells treated with only LPS served as controls for the evaluation of those treated with both LPS and DJ-X-013. The percentage of cell viability was calculated using the equation below:

$$Cell viability ( \% ) = ( {A}_{570} of Treated cells - Blank / {A}_{570} of Untreated cells - Blank ) \times 100$$

### Phase Contrast Imaging and Morphometric Analysis

Cultured RAW264.7 cell morphology of control, LPS, 10 μM, and 50 μM of DJ-X-013 treated cells were documented using an inverted phase contrast microscope (objective: 40×, final magnification 400×; AMG EVOS FL Life Technologies, Carlsbad, CA, USA). Morphometric analysis was performed on 40× images using ImageJ software (NIH). Ten representative images were selected from three experiments for each group and 5 to 8 cells per image were annotated using ImageJ software (NIH), for a total of 75 cells analyzed per group. Each cell was assessed for six parameters, i.e., cell area, perimeter, major axis length, minor axis length, aspect ratio (major axis/minor axis), and circularity [4π×(Area/Perimeter²)].

### Scratch Assay.

Cell mobility was assessed using a scratch or wound healing assay. RAW264.7 macrophages were seeded in a 24-well plate, and incubated for 24 h, then the cell monolayer was scrapped in a straight line to create a scratch with a p200 pipet tip. Debris and detached cells were removed by washing with DMEM medium and an initial (0 h) image was photographed using an inverted phase contrast microscope (10× objective; AMG EVOS FL, Life Technologies). The cells were immediately treated with LPS and DJ-X-013, incubated at 37° C., and imaged in the same location at 4 h, 8 h, 24 h, and 28 h after treatment. The width of the wound was measured at each time point using ImageJ software (NIH) and the cell migration rate was calculated using the following equation:

$${R}_{M} = \frac{{W}_{i} - {W}_{f}}{t}$$

where R_{M} is the Rate of cell migration (μm/h), Wᵢ is the initial wound width at 0 h (μm), W_{f} is the final wound width (μm) at each time point, and t is the duration time (h).

### Immunofluorescence (IF) Staining and Confocal Imaging

After 24 h of treatment with LPS in the absence or presence of DJ-X-013 treatment as described above, RAW264.7 cells were fixed in 4% paraformaldehyde (Cat. no. J19943-K2, Thermo Fisher Scientific, Waltham, MA, USA) and permeabilized with 0.1% Triton X-100. Cells were blocked with 10% goat serum in 1% bovine serum albumin (BSA) and then incubated with rabbit primary antibodies against mouse α-tubulin (Cat. no. A11126, Invitrogen) overnight at 4° C. After washing with PBS, the cells were subsequently incubated with Alexa fluor 488-conjugated goat anti-rabbit secondary antibodies (Cat. no. A-11001, Invitrogen) at RT for 1 h. The cells were also stained with Texas red×phalloidin (Cat. no. T7471, Invitrogen) and counterstained with 4,6-diamidino-2-phenylindole (DAPI) (Sigma-Aldrich, St. Louis, MO, USA). Unbound reagents and antibodies were removed by washing with PBS at every step. Stained cells were mounted with ProLong™ Diamond Antifade mounting media (Cat. no. P36965, Invitrogen), allowed to dry, and microphotographs were grabbed using a Zeiss 710 confocal microscope (objective: 40×) in the institutional core facility.

### Multiplex Assay

Milliplex MAP Multi-Pathway Total Magnetic Bead 9-plex Cell Signaling Multiplex Assay kit (Cat. no. 48-681MAG, Millipore Sigma, USA) was used to determine the total amounts of the following proteins in lysates of RAW264.7 cells treated with LPS in the absence or presence of DJ-X-013: cAMP response element-binding protein (CREB), p38, extracellular signal-regulated kinase/mitogen-activated protein kinase 1/2 (ERK/MAP1/2), STAT5, STAT3, NF-κB, RAC-α serine/threonine-protein kinase (AKT1), cJun N-terminal kinase (JNK), and ribosomal protein S6 kinase B1 (p70-S6k), as directed by the manufacturer. Milliplex MAP Mouse Cytokine/Chemokine Magnetic Bead Panel Premixed 25 Plex Immunology Multiplex Assay kit (Cat. no. MCYTOMAG-70K-PMX, Millipore Sigma) was also used to analyze serum samples from mice with experimental colitis (described below). Briefly, each well of the assay plate was wetted with buffer and 25 μl beads were added to each. The blank well received 25 μl of assay buffer, while each sample well received a serum and the plate was shaken in the dark at 4° C. overnight. The next day, each well was washed twice, 25 μl detection antibody was added to each well, and the plate was shaken in the dark at RT for 1 hr. 25 μl streptavidin-phycoerythrin (PE) was added to each well, the plate was shaken in the dark at RT for 15 min, then amplification buffer was added and the plate was incubated as above for an additional 15 min. After the removal of streptavidin PE/amplification buffer, beads were resuspended in 150 μl assay buffer and analyzed using a Luminex™ System (Austin, TX) and software from Bio-Rad (Hercules, CA). The results were expressed as either mean fluorescence intensity (MFI) or picograms per milliliter (pg/ml).

### Animal Experiments

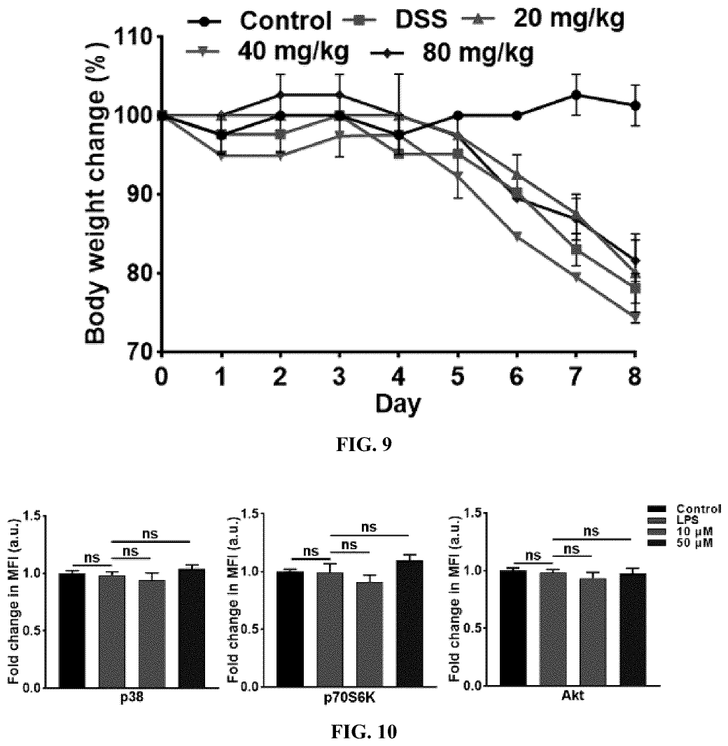

All animal experimentation was performed under protocol no. 23-0450 approved by the University of Tennessee Health Science Center (UTHSC) Institutional Animal Care and Use Committee (IACUC). Wild-type (WT) C57BL/6 female mice (8 weeks old) were purchased from Jackson Laboratories (Bar Harbor, ME, USA) and housed in a specific pathogen-free animal facility at UTHSC, Memphis with normal 12/12 h light/dark cycles. The mice were housed for a week for acclimatization to the animal facility before starting the experiment. Following one week of acclimatization, the mice were randomly divided into three experimental groups, each containing five mice (n=5/group): control, DSS alone, and DSS+DJ-X-013 (designated DJ-X-013 throughout the experiment). DSS was used to induce experimental colitis in the mice, as described.²⁷ Briefly, on the day the experiment started (day 0), the DSS and DJ-X-013 groups were provided DSS (Molecular weight: 36-50 kDa; Cat. no. 160110, MP Biomedicals, Santa Ana, CA, USA) at 3.5% in the drinking water, which was changed at two-day intervals, and the treatment was continued up to day 6 for a total of 7 days. Thereafter, the DSS water was replaced with plain drinking water. In a preliminary dose-response experiment using DJ-X-013 at doses of 20, 40, and 80 mg/kg body weight, the 20 mg/kg dose effectively reduced colitis symptoms relative to the higher doses (FIG. 9). Therefore, 20 mg/kg dose of DJ-X-013 were used for this study. Mice in the DJ-X-013 group were administered daily doses of 20 mg/kg DJ-X-013 (dissolved in a standardized ratio of DMSO, ethanol, and PBS) in 100 μl volume via oral gavage on day 1 and continuing to day 7. During days 1-7, the control and DSS alone groups were administered 100 μl vehicle (same ratio of solvents). The mice were monitored daily for body weight, behavior, and clinical symptoms of colitis (diarrhea, stool consistency, and blood in fecal matter). The mice were euthanized at the experimental endpoint on day 8 and spleen, mesenteric lymph nodes (MLNs), colon tissues, and whole blood (from which serum was collected by centrifugation) were collected for further downstream experiments. The experiment was repeated three times to achieve statistical significance.

Single-Cell Isolation from Spleen and Mesenteric Lymph Nodes (MLNs)

After removing the fat bodies, the spleen and MLNs were collected in ice-cold complete Roswell Park Memorial Institute Medium 1640 (RPMI 1640; Cat. no. 10-041-CV, Corning). Tissues were homogenized for 30-45 s. in a Seward™ Stomacher™ 80 lab blender (Fisher Scientific), the cell suspension was passed through a 70 μm filter, and the cells were collected by centrifugation (300×g) at 4° C. for 10 min. MLNs cells were resuspended in RPMI media and the total cell number and percentage of live cells were estimated in the Invitrogen cell counter after staining with trypan blue. Spleen cells were incubated with red blood corpuscle lysis buffer (Cat. no. 00-4333-57, Invitrogen) for 3-4 min, resuspended in complete RPMI, and the cells were collected by centrifugation (300×g) at 4° C. for 10 min. The cell pellet was resuspended in complete RPMI, and the cells were passed through a 70 μm filter and counted as described above.

Lymphocyte Isolation from Colon Lamina Propria (LP)

Total lymphocytes were isolated from the colon lamina propria using a mouse lamina propria dissociation kit (Cat. no. 130-097-410, Miltenyi Biotec, Auburn, CA, USA) according to the manufacturer's protocol. Briefly, the colons were collected and cleaned in Hank's balanced salt solution (HBSS). Colons were cut longitudinally, then transversely into small pieces (approx. 0.5 cm in length), placed 50 ml falcon tube and incubated at 37° C. in first pre-digestion solution (2×20 min.), then HBSS (20 min.), then digestion solution containing enzymes A, D, and R (30 min.) in a MACS™ C tube (Cat. no. 130-093-237, Miltenyi Biotec), in a MACSmix tube rotator with continuous rotation. The samples were transferred to a gentle MACS™ Dissociator (Cat. no. 130-093-235, Miltenyi Biotec) and dissociated using the gentle MACS program “m_intestine_01”. After a brief centrifugation to collect the cells, the cell pellet was resuspended in PB buffer (PBS with 0.5% bovine serum albumin), and the cells were passed through a 100 μm filter. The cells were collected by centrifugation, resuspended in a complete RPMI medium, and counted in a counting chamber after trypan blue staining before further analysis.

### Flow Cytometry

Cultured RAW 264.7 cells or cells isolated from mouse spleen, MLN, or LP were washed and resuspended in ice-cold flow cytometry staining buffer (FACS buffer; PBS with 1% FBS). For surface staining, the manufacturer's recommended concentration of the appropriate antibodies was added to the cells and incubated in the dark at 4° C. for 40 min. with occasional shaking. Details of flow antibodies are described in Table 1; all fluorescence-conjugated antibodies and buffers used for intracellular staining were purchased from Biolegend (San Diego, CA). For intracellular staining, the cells were fixed and permeabilized sequentially in fixation/permeabilization buffer, then permeabilization buffer, the appropriate antibodies were added, and the cells were incubated at RT for 30 min. After removal of unbound antibodies by washing, cells were resuspended in 300 μl FACS buffer and analyzed in a Novocyte flow cytometer (Agilent Technologies, Santa Clara, CA).

TABLE 1
List of flow cytometry Anti-mouse antibody from BioLegend Inc.

| Name of the | Catalogue | Conjugated | Concentration used | | |
|---|---|---|---|---|---|
| protein | no. | fluorophore | for 10⁶ cells | | Host |
| TNF-α | 506308 | APC | 0.25 | μg | Rat |
| iNOS | 696806 | PE | 0.125 | μg | Rat |
| CD11b | 101208 | PE | 0.25 | μg | Rat |
| CD11c | 117311 | AF488 | 0.25 | μg | Armenian Hamster |
| Gr-1 | 108412 | APC | 0.25 | μg | Rat |
| Ly6c | 128046 | APC Fire 750 | 0.5 | μg | Rat |
| CD3 | 100204 | FITC | 1 | μg | Rat |
| CD4 | 100412 | APC | 0.25 | μg | Rat |
| CXCR3 | 126506 | PE | 0.25 | μg | Armenian Hamster |
| NK1.1 | 108708 | PE | 0.25 | μg | Mouse |
| IL17A | 506926 | BV421 | 0.25 | μg | Rat |

### RNA Isolation and Reverse Transcription-Quantitative Polymerase Chain Reaction (RT-qPCR)

Total RNA was extracted from RAW 264.7 cells and colon tissue cells using an RNeasy Mini kit (Cat. no. 74104, QIAGEN, Germantown, MD, USA) following the manufacturer's protocol. The concentration and purity of the RNA were determined using a Nanodrop spectrophotometer (Fisher Scientific). For each sample, 250 ng of extracted RNA was used as a template by reverse transcriptase for cDNA synthesis using an iScript cDNA synthesis kit (Cat. no. 1708891, Bio-Rad) according to the manufacturer's protocol. This cDNA was subsequently used as a template for qPCR using iTaq Universal SYBR Green Supermix (Cat. no. 1725121, Bio-Rad). All primers were purchased from Integrated DNA Technologies (IDT; Coralville, IA, USA).

### Immunoblot (IB) Analysis

RAW 264.7 cells and cells isolated from colon tissue were lysed with radioimmunoprecipitation assay (RIPA) buffer (Cat. no. J63306, Alfa Aesar, Ward Hill, MA, USA) supplemented with Halt™ protease and phosphatase inhibitor cocktail (Cat. no. 78442, Thermo Scientific). Complete cell lysis was achieved by vigorous pipetting, tissue homogenization, sonication, and incubation on ice for 30 min. Cell debris was removed by centrifugation at 16,000×g at 4° C. for 20 min. and the total protein concentration of the supernatant was estimated using a Pierce™ BCA Protein Assay Kit (Cat. no. 23225, Thermo Fisher Scientific). Equal amounts (20 μg) of protein for each sample were loaded and separated on a 10% sodium dodecyl sulfate-polyacrylamide gel electrophoresis (SDS-PAGE) gel cast using a TGX™ FastCast™ Acrylamide Starter Kit (Cat. no. 1610172, Bio-Rad) and were transferred to polyvinylidene fluoride (PVDF) membranes (Cat. no. 1620177, Bio-Rad) using a Trans-Blot Turbo Transfer System (Cat. no. 1704150, Bio-Rad). The membrane was blocked with blocking buffer (Cat. no. 927-60001, LI-COR Biosciences, Lincoln, NE, USA) at RT for 2 h and incubated with the following primary antibodies at 4° C. overnight with shaking: NF-κB p65 (1:1000; Cat. no. 8242T, Cell Signaling Technology, Danvers, MA, USA) or NF-κB p100-50 1:1000; Cat. no. 48828, Cell Signaling Technology). Antibodies against the housekeeping protein β-actin (1:5000; Cat. no. 926-42212, LI-COR) were used as a loading control. Unbound primary antibodies were removed by washing with Tris-buffered saline (TBS) containing Tween® 20 (polyethylene glycol sorbitan monolaurate) and the membranes were incubated in the dark at RT for 1 h with the following IRDye® 800CW-conjugated secondary antibodies: goat anti-mouse IgG (1:5000; Cat. no. 926-32210, LI-COR Biosciences) or goat anti-rabbit IgG (1:5000; Cat. no. 926-32211, LI-COR Biosciences). After washing to remove excess secondary antibodies, the membrane was visualized and imaged using a LICOR Odyssey® DLX imaging system (LI-COR Biosciences). Densitometric analysis was performed using ImageJ software (NIH).

### Hematoxylin and Eosin Stain (H&E), Imaging and Measurement of Colon Layers Width

Mice distal colons were fixed using 4% paraformaldehyde (Cat. no. J19943-K2, Thermo Scientific) for 24 h and embedded in paraffin. Fixed tissues were cut into 5 μm sections, deparaffinized, stained with hematoxylin and eosin (H&E), and examined using a bright field microscope (Model no. BX43, Olympus Life Science Solutions/Evident Scientific, Tokyo, Japan) in different magnifications. The inflammation score was estimated based on the severity of epithelial cell disruption, loss of crypt structure, loss of goblet cells, and the degree of inflammatory cell infiltration. The inflammatory state of each colon was characterized and scored as follows: having no change when compared with tissue samples from control mice (score=0); having a few mononuclear cell infiltrates (score=1); having minimal mononuclear cells (score=2); having a medium level of infiltration (score=3); or exhibiting loss of epithelial cells with heavy cellular infiltrates in the sub-mucosa (score=4). Furthermore, colon epithelial folding length and sub-mucosal width were semi-quantitatively estimated from H&E images using ImageJ software (NIH).

### Statistical Analysis

All data are shown as mean values±standard error of the mean (SEM). Statistical analysis was performed using one-way analysis of variance (ANOVA) followed by either Tukey's or Dunnett's multiple comparison tests (as indicated in the figure legends) to determine the significance level. A p-value of 0.05 was considered the level of significance in all analyses [ns (not significant) p>0.05, *p<0.05, **p<0.01, ***p<0.001 and ****p<0.0001]. Graphical representations were generated using GraphPad Prism software (GraphPad Software, Boston, MA).

### Computer-Aided Prediction of Anti-Inflammatory Properties of DJ-X-025

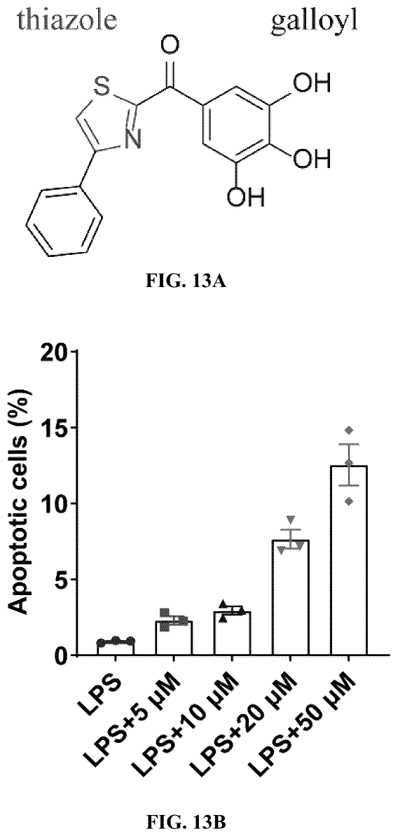

The properties of DJ-X-025, a molecule whose structure (FIG. 13A) is comprised of a thiazole ring connected to a galloyl group were examined (FIG. 21). The molecule demonstrated its beneficial anti-inflammatory activity in the acute inflammatory model and provided a computer-aided prediction as a drug option for suppressing inflammation. DJ-X-025 was evaluated using the computational methods provided by the SwissADME programs (www.swissadme.ch, Molecular Modeling Group-Swiss Institute of Bioinformatics, Lausanne, CH).

### DJ-X-025 Treatment to LPS-Induced RAW 264.7 Macrophages and MC38 Cells

RAW 264.7 macrophages were cultured for 24 hr in DMEM and stimulated with 100 ng/ml LPS (Cat. no. L4391, Millipore Sigma, St. Louis, MO). After 24 hr cells were treated with 5, 10, or 20 μM DJ-X-025 for a further 24 hr. Next, the cells were analyzed as a control group (without LPS), LPS alone, and LPS with various concentrations of the DJ-X-025 treatment group. In the initial experiments, 5 μM DJ-X-025 was included but did not result in any significant changes, thus this dose was not included in further experiments. All further studies were conducted using 10 μM and 20 μM doses of DJ-X-025 in all experiments.

The MC38 mouse colon adenocarcinoma cells (Cat no: ENH204-FP; Kerafast, Boston, MA) were cultured in DMEM supplemented with 10% fetal bovine serum (FBS) in 1% penicillin and streptomycin at 37° C., 5% CO₂ incubator. Cells (3×10⁵) were seeded in a six-well plate overnight and treated with control, 10 μM, and 20 μM DJ-X-025 for 24 h. Phase contrast imaging and total RNA isolation were performed after 24 h incubation.

### Apoptosis Assay

To determine whether DJ-X-025 induces apoptosis in RAW264.7 macrophage, cells were cultured in 6-well plates in complete DMEM at 37° C., 5% CO₂ for 24 hr. The cells were stimulated with (100 ng/ml) dose of LPS and concurrent treatment with 5, 10, 20, and 50 μM of DJ-X-025 and incubated at 37° C. for 24 hr. Apoptosis and cell viability were determined in control, LPS-induced RAW264.7 cells treated with various doses of DJ-X-025 by using fluorescein isothiocyanate-labeled (FITC) Annexin V Apoptosis Detection kit 1 (BD Pharmingen, Material no. 556547) following the manufacturer's protocol. In brief, cells were harvested and washed twice with cold PBS and resuspended in 1× binding buffer, and 5 μl of FITC Annexin V and 5 μl of Propidium Iodide were added in 10⁵ cells in 100 μl cell suspension. Cells were gently vortexed and incubated for 15 min at room temperature in the dark. The cells were analyzed using Novocyte flow cytometry analysis within 1 hour with 400 μl of 1× binding buffer. The data were presented as a percentage of RAW264.7 macrophages apoptotic cells after DJ-X-025 treatment.

### Morphometric Analysis Using Phase-Contrast Images

RAW264.7 macrophages and MC38 cell morphology were documented in a phase-contrast microscope. The morphology of RAW264.7 macrophages in control, LPS alone, LPS with 5 μM, 10 μM, and 20 μM DJ-X-025 treated group was determined. Ten images were selected from 3 experiments from each group and analyzed them by using ImageJ software. More than 125 cells per group were analyzed and each cell was measured for various parameters viz. cell area, perimeter, circularity, etc.

### Immunofluorescence (IF) Staining for Confocal Microscope

RAW264.7 macrophages were incubated for 24 hr 37° C. without/with LPS and 5, 10, or 20 μM doses of DJ-X-025 treatment. Next, the cells were fixed with 4% paraformaldehyde and permeabilized with 0.1% Triton X-100. After fixation, permeabilization, and blocking, RAW264.7 macrophages were incubated at 4° C. overnight with rabbit primary antibodies specific for α-tubulin and subsequently incubated with Alexa fluor 488-conjugated goat anti-rabbit secondary antibodies at RT for 1 hr. The macrophages were stained with Texas red×phalloidin and counterstained with 4,6-diamidino-2-phenylindole (DAPI) (Sigma-Aldrich, St. Louis, MO, USA) to visualize each cell and its nucleus. A Zeiss 710 confocal microscope (objective: 40×) was used at the UTHSC core facility to grab microphotographs of these cells.

### RNA Isolation and Reverse Transcription-Quantitative Polymerase Chain Reaction (RT-qPCR)

The total RNA was isolated from the colon tissue RAW 264.7 cells and MC38 cells which were obtained from in vivo and in vitro experiments separately using the RNeasy Mini kit following the manufacturer's protocol. The purity and concentration of RNA were measured using a Nanodrop spectrophotometer and 500 ng of extracted RNA was used as a template for cDNA synthesis by reverse transcriptase using an iScript cDNA synthesis kit. Primers were purchased from Integrated DNA Technologies (IDT; Coralville, IA, USA).

### Animal Studies

Female C57BL/6 mice (6 to 8 weeks of age) were purchased from Jackson Laboratories (Bar Harbor, ME, USA). All mice were kept for 1 week in normal housing conditions for acclimatization in a pathogen-free animal facility at the University of Tennessee Health Science Center (UTHSC) Memphis in normal light/dark cycles. All experiments were conducted under approved protocol no. 23-0450, by the UTHSC Institutional Animal Care and Use Committee (IACUC). After 1 week, mice were randomly divided into control, DSS, and DSS+DJ-X-025 experimental groups containing five mice each (N=5). The control group mice received normal water on day 0. The last two groups of mice (DSS, and DSS+DJ-X-025) were provided 3.5% DSS in drinking water similarly as described in the previous study [19]. Doses of 20, 40, and 80 mg/kg were used, and the 20 mg dose showed the best efficacy (body weight change), without toxicity, and better results to ameliorate colitis. The same dose was used in the in vivo study. The treatment group of mice was administered as daily doses of 100 μl of 20 mg/kg DJ-X-025 (based on a preliminary study for the most effective dose, dissolved in a standardized ratio of DMSO, ethanol, and PBS) via oral gavage till day 7. The control and DSS group of mice were orally administered 100 μl vehicle (using the same ratio of solvents). The body weight, behavior, and clinical symptoms of colitis (diarrhea, stool consistency, and blood in fecal matter) of mice were monitored daily till the experimental endpoint. The mice were sacrificed on day 8 and blood was isolated for further systemic cytokine and chemokine analysis. The spleen, MLNs, and colon tissues were collected for single-cell isolation and further downstream flow cytometry analysis. The colon tissues from each mouse were also preserved for histology, RT-qPCR, and Western analysis. All mice experiments were repeated three times to achieve statistical significance.

### Splenocytes and Mesenteric Lymph Nodes (MLNs) Cells Isolation

Spleen and MLNs were stored in a complete RPMI medium and isolated a single-cell suspension. Spleen cells were incubated with red blood cell lysis buffer, washed with media, and the cell pellet was resuspended in complete media after passing through a 70 μm filter. The number and percentage of live cells in the spleen and MLNs were enumerated after trypan blue staining using an Invitrogen cell counter. The cells were maintained in complete media on ice for further analysis.

### Isolation of Colon Lamina Propria (LP) Lymphocytes

Entire lymphocytes from the colon lamina propria were separated using a mouse LP dissociation kit. Colons were collected and fecal matter was cleaned in an HBSS medium. Next, colons were cut into small pieces and placed in a gentleMACS™ C tube for isolation of colon lymphocytes following the manufacturer's protocol. Finally, the LP lymphocyte cells were collected by centrifugation, stained with trypan blue staining, and counted in an Invitrogen cell counter for further analysis.

### Flow Cytometry

The flow cytometry analysis was performed in RAW 264.7 macrophage (in vitro) and cells isolated from spleen, MLNs, and colon LP (in vivo) after DJ-X-025 treatment. The cells were washed with PBS and resuspended in staining buffer for flow cytometry analysis. All flow cytometry antibodies were from Biolegend (San Diego, CA) as described in detail in Table 3. Buffers used for intracellular staining were also purchased from BioLegend. The recommended concentration of the appropriate antibodies was added to the 10⁶ cells in a flow tube and incubated them in the dark at 4° C. for 40 min with occasional shaking. Next, cells were fixed and permeabilized sequentially in fixation/permeabilization buffer for intracellular staining as per manufacturer protocol (Biolegend, San Diego, CA, USA). The cells were incubated with the appropriate intracellular fluorescence-labeled antibodies at RT for 30 min in the dark. The cells were washed and resuspended in 300 μl FACS buffer and analyzed in a Novocyte flow cytometer (Agilent Technologies, Santa Clara, CA).

TABLE 3
List of flowcytometry Anti-mouse antibody from BioLegend.

| Name of the | Conjugated | BioLegend | Concentration used | | |
|---|---|---|---|---|---|
| marker | fluorophore | Cat. no. | for 10⁶ cells | | Host |
| CD11b | PE | 101208 | 0.25 | μg | Rat |
| CD11c | BV421 | 117329 | 0.25 | μg | Armenian Hamster |
| CD4 | APC | 100412 | 0.25 | μg | Rat |
| CXCR3 | PE | 126506 | 0.25 | μg | Armenian Hamster |
| F4/80 | BV421 | 123137 | 0.25 | μg | Rat |
| FOXP3 | PE | 126404 | 0.6 | μg | Rat |
| IL17A | BV421 | 506926 | 0.25 | μg | Rat |
| iNOS | PE | 696806 | 0.125 | μg | Rat |
| Ly6C | FITC | 128006 | 0.25 | μg | Rat |
| TNF-α | APC | 506308 | 0.25 | μg | Rat |

Antibodies used for flow cytometry were purchased from BioLegend, Inc. (San Diego, CA, USA).
Abbreviations used: AF, Alexa fluor; APC, Allophycocyanin; BV, Brilliant violet; CD, Cluster of differentiation; CXCR3, CXC motif chemokine receptor 3; FITC, Fluorescein isothiocyanate; FOXP3, Forkhead box P3; IL, Interleukin; iNOS, Inducible nitric oxide synthase; Ly6C, lymphocyte antigen 6 family member C1; PE, Phycoerythrin; TNF-a, Tumor necrosis factor-alpha.

### Hematoxylin and Eosin (H&E) Staining for Bright Field Microscopic Imaging, and Analysis

After cleaning the fecal matter, a portion of the distal colon was fixed in 4% paraformaldehyde for 24 hr, embedded in paraffin, and cut into 5 μm thin sections. The colon sections were deparaffinized, stained with hematoxylin and eosin (H& E), and examined using a bright field microscope. Based on the epithelial cell disruption, loss of goblet cells, and the frequency of inflammatory cell infiltration, we estimated the inflammation score from twenty selected fields from three experiments. Colon inflammatory scores were defined based on a field having no change to a few mononuclear cell infiltrates (score=0-1), having more than 10 cell infiltrates in the colon (score=2-4), having several multifocal cellular infiltrates level of infiltration (score=4-6), or exhibiting loss of epithelial cells with heavy cellular infiltrates in the sub-mucosa (score=6-8). Furthermore, the width of the colon muscularis mucosae muscle layer was estimated semi-quantitatively from twenty fields and a total of 200 measurements of H&E images were analyzed by using ImageJ software.

### Inflammatory Cytokines and Chemokines Multiplex ELISA Assay

A Mouse ProcartaPlex™ Mix & Match 22-plex (Cat. No. PPX-22-MXGZHGC, Thermo Fisher Scientific, USA) was used in a 96-well plate configuration to determine levels of inflammatory cytokines and chemokines in serum from mice with experimental colitis as per manufacturer's protocol. Briefly, different dilutions of a standard antigen solution were prepared as per the suggested protocol from (Thermo Fisher Scientific, USA). Capture beads (50 μl) were added to each well of the assay plate, followed by 25 μl of platinum assay buffer and 25 μl antigen standard or serum samples. The blank well received only 50 μl of platinum buffer. The plate was incubated in the dark with shaking at 4° C. overnight. The following day, the plate was washed and incubated with detection antibodies for 1 h and streptavidin-phycoerythrin (PE) for 30 min at RT. After washing, the beads were resuspended in the recommended buffer and analyzed using a Luminex™ System (Austin, TX) and software from Bio-Rad (Hercules, CA). The results were expressed as picograms per milliliter (pg/ml).

### Western Blot (WB) Analysis of Proteins in Colon Tissue

Colon tissues were lysed with radioimmunoprecipitation assay (RIPA) buffer (Cat. no. J63306, Alfa Aesar, Ward Hill, MA, USA) added with Halt™ protease and phosphatase inhibitor cocktail as described in manufacturer protocol. Cell lysis was performed using a handheld tissue homogenizer and sonicator and incubated the lysate on ice for 30 min. The lysate was centrifuged to remove any cell debris and the protein concentration of the supernatant was estimated using a Pierce™ BCA Protein Assay Kit (Cat. no. 23225, Thermo Fisher Scientific). Equal amounts of protein (25 μg) for each sample were loaded on a 10% gel and run by sodium dodecyl sulfate-polyacrylamide gel electrophoresis (SDS-PAGE). The segregated proteins were transferred to polyvinylidene fluoride (PVDF) membranes. The membranes were blocked with blocking buffer for 1 h at RT and incubated with primary antibodies at 4° C. overnight with constant shaking: phospho (Tyr705)-STAT3 (1:1000; Cat. no. 9145S, Cell Signaling Technology, Danvers, MA, USA), NF-κB p65 (1:1000; Cat. no. 8242S, Cell Signaling Technology,) and occludin (1:5000; Cat. no. 27260-1-AP, Proteintech, Rosemont, USA). β-actin was used as a housekeeping reference protein and antibodies specific for the β-actin (1:5000; Cat. no. 926-42212, LI-COR Biosciences) were incubated similarly. Unbound primary antibodies were washed with Tris-buffered saline (TBS) containing Tween® 20 (polyethylene glycol sorbitan monolaurate) and the membranes were incubated in the dark at RT for 1 h with the following secondary antibodies: goat anti-rabbit IgG (1:5000; Cat. no. 926-32211, LI-COR Biosciences) and/or goat anti-mouse IgG (1:5000; Cat. no. 926-68070, LI-COR Biosciences). After washing, the membrane was scanned and imaged using a LICOR Odyssey® DLX imaging system (LI-COR Biosciences). Protein bands' optical density relative to β-actin was analyzed using ImageJ software (NIH).

### Statistical Analysis

All data are presented as mean values±standard error of the mean (SEM) for three experiments. A one-way analysis of variance (ANOVA) was performed followed by either Tukey's or Dunnett's multiple comparison statistical analysis tests where appropriate to determine the significance level. A p-value of 0.05 was considered to be statistically significant in all analyses (*p<0.05, **p<0.01, ***p<0.001 and ****p<0.0001). All graphical representations were generated using GraphPad Prism software (GraphPad Software, Boston, MA) provided by the Molecular Bioinformatics core center at UTHSC.

### Abbreviations

ADME, Absorption distribution, metabolism and excretion; AKT, Rac-alpha serine/threonine-protein kinase; ANOVA, Analysis of variance; BBB, Blood-brain barrier; CD, Crohn's disease; CREB, cAMP response element-binding protein; CXCR3, C-X-C Motif Chemokine Receptor 3; DAPI, 4,6-diamidino-2-phenylindole; DC, dendritic cell; DMEM, Dulbecco's modified essential medium; DMSO, dimethyl sulfoxide; DSS, dextran sodium sulfate; ERK/MAK Extracellular signal-regulated kinase/Mitogen-activated kinase; FBS, Fetal bovine serum; GI, Gastrointestinal; H&E, hematoxylin and eosin stain; HA, Number of heavy atoms; HBA, Number of hydrogen-bond acceptors; HBSS, Hanks' balanced salt solution; HIA, Human intestinal absorption; IB, Immunoblot, IBD, Inflammatory bowel disease; IF, Immunofluorescence; IFN-γ, Interferon gamma; IL, Interleukin; iNOS, Inducible nitric oxide synthase; JNK, cJun N-terminal kinase; LP, Lamina propria; LPS, Lipopolysaccharide; MDSC, Myeloid-derived suppressor cell; MFI, Mean fluorescence intensity; MLN, Mesenteric lymph node; MLOGP, Moriguchi octanol-water partition coefficient; MT, Microtubule; MTT, 3-[4,5-dimethylthiazol-2-yl]-2,5 diphenyl tetrazolium bromide; MW, Molecular weight; NF-κB, Nuclear factor kappa-light-chain-enhancer of activated B cells; NK cell, Natural killer cell; NKT, Natural killer T cells; PBS, Phosphate-buffered saline; RB, Number of rotatable bonds; RT-qPCR: Reverse transcription-quantitative polymerase chain reaction; RPMI medium, Roswell Park Memorial Institute Medium; RT, Room temperature; SEM, Standard error of the mean; STAT, Signal transducer and activator of transcription; Th cell, T helper cell; TNF-α, Tumor necrosis factor alpha; UC, Ulcerative colitis. The following examples further illustrate aspects of the present invention. However, they are in no way a limitation of the teachings or disclosure of the present invention as set forth herein.

### Example 1

### Synthesis of DJ-X-013

The synthesis of DJ-X-013 was prepared through two methods as below showed in Scheme 1.

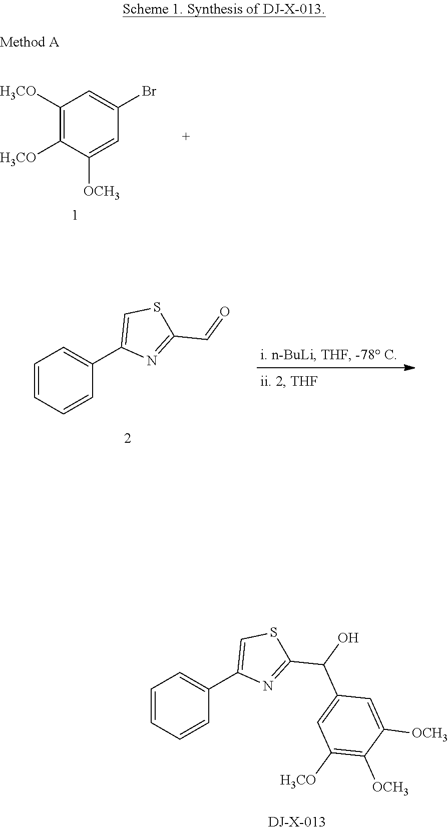 [SMILES: COc1cc(Br)cc(OC)c1OC ; O=Cc1nc(-c2ccccc2)cs1 ; COc1cc(C(O)c2nc(-c3ccccc3)cs2)cc(OC)c1OC]

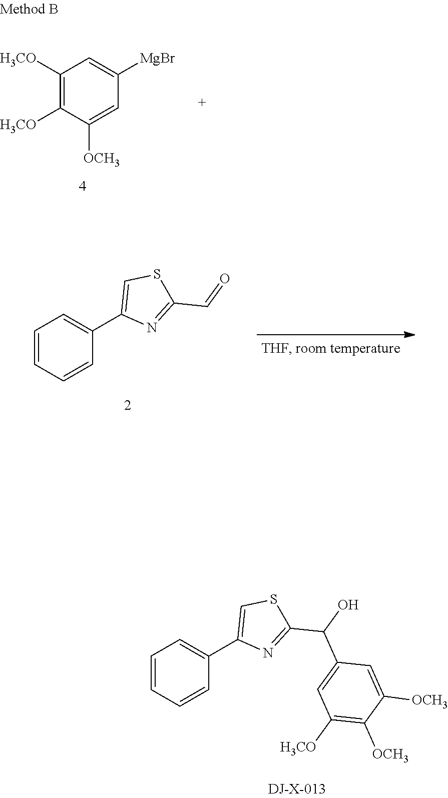

Method A. A solution of n-butyllithium in hexane (2.5 M, 1.2 mL, 3 mmol) was added dropwise to a solution of 5-bromo-1,2,3-trimethoxybenzene (1, 741 mg, 3 mmol) in tetrahydrofuran (15 mL) under nitrogen at −78° C. After 10 minutes, a pre-cooled solution (−78° C.) of 4-phenylthiazole-2-carbaldehyde (2, 568 mg, 3 mmol) in tetrahydrofuran (10 mL) was added via a double-tipped needle. The mixture was stirred at −78° C. for 30 min. The cooling bath was removed, and the mixture was allowed to reach ambient temperature under 30 min. The solvent was removed in vacuo, and the residue was partitioned between ethyl acetate and water. The phases were separated, and the organic layer was washed with brine, dried (MgSO₄) and the solvent was removed in vacuo. The crude product was purified by column chromatography on silica gel using hexane/ethyl acetate (70:30, v/v) as the eluent to provide designed compound as a yellow solid (DJ-X-013). Yield=74%. Purity (LC, tₘᵢₙ=3.59) 99.70%; UV λmax 254.45 nm, MS (ESI) m/z 358.10 [M+H]⁺; LCMS (ESI) m/z calcd for C₁₉H₁₉NO₄S 358.1113 [M+H]⁺; found: 358.1104 [M+H]⁺, 340.1000 [M−H₂O+H]⁺; ¹H NMR (CDCl₃, 400 MHz) δ 7.89 (d, J=7.2 Hz, 2H, ArH), 7.45 (d, J=7.2 Hz, 2H, ArH), 7.42 (s, 1H, ArH), 7.34 (t, J=7.6 Hz, 1H, ArH), 6.76 (s, 2H, ArH), 6.03 (s, 1H, CH—OH), 3.86 (s, 6H, (OCH₃)₂), 3.85 (s, 3H, OCH₃), 1.68 (bs, 1H, OH); ¹³C NMR (CDCl₃, 100 MHz) δ 173.55, 154.92, 153.46 (2C), 137.91, 136.79, 134.10, 128.80 (2C), 128.30, 126.32 (2C), 103.51 (2C), 73.92, 60.87, 56.15 (2C).

Method B. To a solution of 4-phenylthiazole-2-carbaldehyde (2, 0.568 g, 3 mmol) in 20 mL THF was slowly added a THF solution of 3,4,5-trimethoxyphenylmagnesiumbromide (4, 0.5 N, 6.0 mL, 3 mmol) at 0° C. The mixture was allowed to stir for 2 hr. until disappeared starting aldehyde 2 and quenched with saturated NH₄Cl, extracted with ethyl ether, dried with anhydrous MgSO₄. The solvent was removed under reduced pressure to yield a crude product, which was purified by column chromatography to obtain pure target compound (DJ-X-013). Yield 72.9%.

### Example 2

### Determination of DJ-X-013 ADME Properties for Gastrointestinal Absorption, Blood-Brain Barrier Penetration and Physical Properties

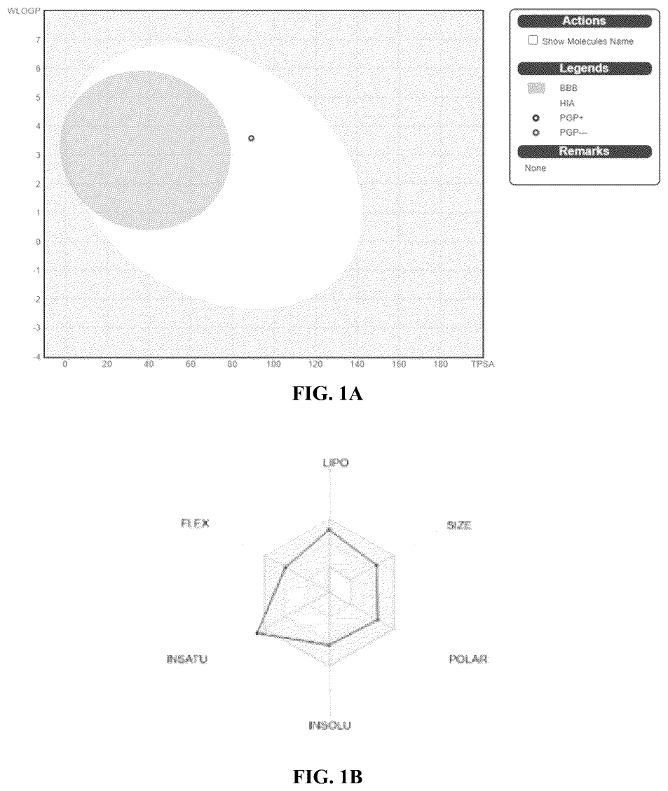

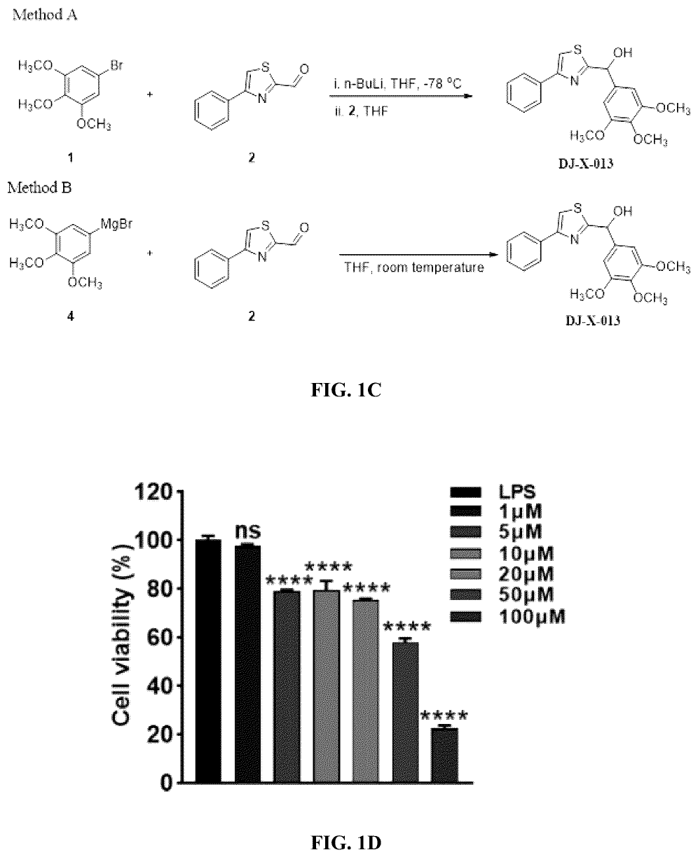

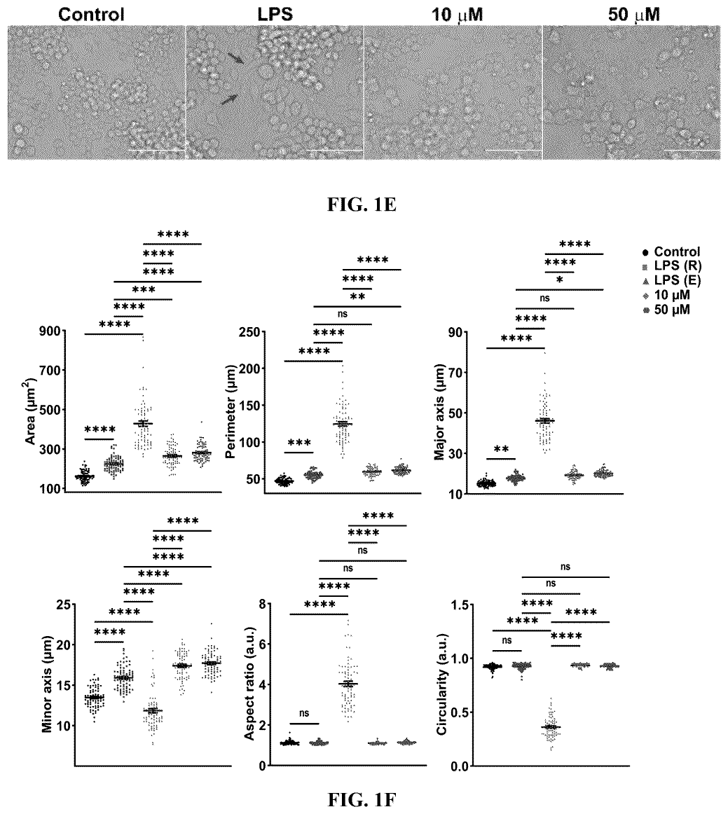

DJ-X-013 was evaluated through computational methods named the SwissADME program.³⁹ The DJ-X-013 calculated and expected ADME properties and other drug-likenesses are presented in FIG. 1A and FIG. 1B. The BOILED-Egg model (FIG. 1A) allows simultaneous prediction of passive gastrointestinal (GI) adsorption and brain penetration, with the BBB permeation region represented as a round, yellow yolk-like sphere, the human intestinal absorption (HIA) region as a white oval, and the low absorption and limited brain permeation region as a grey rectangle.³⁹ In this image (FIG. 1A), the queried molecule DJ-X-013 is shown within the HIA as a red hollow sphere whose color and location indicate that it is negative for P-glycoprotein (PGP). Thus, this molecule is predicted to lack penetration of the BBB and to avoid the pitfalls of low absorption and limited brain permeation.

The two-dimensional (2D) structure of DJ-X-013 and its classification according to the canonical simplified molecular-input line-entry system (SMILES) code is shown in the left panel of FIG. 1B, while its physicochemical properties are shown in the right panel. The pink shaded area at right of FIG. 1B represents the physicochemical space that is suitable for oral bioavailability, while DJ-X-013 is shown as a red line, calculated from its physical properties, which were assessed based on its predicted lipophilicity (LIPO), size (SIZE), polarity (POLAR), insolubility (INSOLU), insaturation (INSATU), and flexibility (FLEX). The key physicochemical, pharmaceutical, and drug-likeness properties of DJ-X-013 are summarized in Table 2. DJ-X-013 was predicted to have high GI absorption, no penetration of the BBB, and high bioavailability. Traditionally, small-molecule drugs with therapeutic activity comply with Lipinski's Rules of Five,⁴¹ i.e., having molecular mass less than 500 Da, fewer than five hydrogen-bond donors, fewer than ten hydrogen-bond acceptors, and an octanol-water partition coefficient (log P_{o/w}) less than 5. By these criteria (Table 2), DJ-X-013 was predicted to have therapeutic activity. Finally, we used two different techniques, methods A and B, to synthesize DJ-X-013, as detailed in Materials and Methods and summarized in FIG. 1C. The final product of both synthesis methods (compound DJ-X-013) shared the same physicochemical properties and was tested for its biological activity.

TABLE 2
Predicted physicochemical, pharmacokinetic, and drug-likeness
properties of DJ-X-013.^{a}

| | Physicochemical parameters | | | | | |
|---|---|---|---|---|---|---|
| DJ-X-013 | Formula | MW (g/mol) | HA | RB | HBA | HBD |
| | C₁₉H₁₉NO₄S | 357.42 | 25 | 6 | 5 | 1 |

| | Pharmacokinetic parameters | | |
|---|---|---|---|
| Structure | MLOGP | GI absorption | BBB permeation |
| | 1.29 | High | No |

| 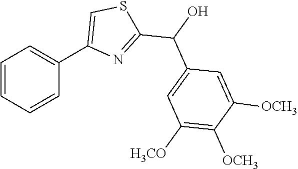 [SMILES: COc1cc(C(O)c2nc(-c3ccccc3)cs2)cc(OC)c1OC] | Drug-likeness properties | |
|---|---|---|
| | Lipinski's Rules of Five | Bioavailability |
| | Yes: 0 violation | 0.55 |

^{a}Properties were determined using SwissADME predictive software.³⁹
Abbreviations used: BBB, Blood-brain barrier; GI, Gastrointestinal; HA, Number of heavy atoms;
HBA, Number of hydrogen-bond acceptors; HBD: Number of H-bond donors; MLOGP,
Moriguchi octanol-water partition coefficient (LogP_{o/w} topological method); MW, Molecular
weight; RB, Number of rotatable bonds.

### Example 3

### DJ-X-013 Alters Cell Viability of Cultured RAW264.7 Macrophages

To determine its effect on cell viability (cytotoxicity), an LPS-activated culture of RAW264.7 macrophages was treated with DJ-X-013 over a range of different concentrations for 24 h and used an MTT assay to examine their percent viability relative to control cells treated with LPS alone (100 ng/ml). When DJ-X-013 was used at a concentration range of 5 to 20 μM, cells treated with LPS and DJ-X-013 retained viability of 79.3±3.8% to 75.3±0.5%, whereas at a concentration of 100 μM, only 22.4±2.1% of cells remained alive after 24 h (FIG. 1D). These results were used in a linear equation (y=mx+c, where x and y are variables, m is the slope, and c is the y-intercept) to calculate an LC₅₀ of 59.5 μM, indicating that treatment of these cells with DJ-X-013 induced 50% cell death at a concentration of approximately 60 μM. Based on these results, we selected concentrations of 10 μM and 50 μM doses for our downstream in vitro experiments.

### Example 4

DJ-X-013 Restores the Morphology of RAW 264.7 Macrophages after LPS Stimulation

RAW 264.7 macrophages without treatment were used as a control in all downstream in vitro experiments including morphometric analysis by phase contrast microscopy (FIG. 1E); these control cells exhibited a morphology that was mostly round. On treatment with LPS alone, these cells exhibited two distinct morphologies: round (R) and elongated (E), the latter of which are indicated by red arrows (FIG. 1E). Interestingly, after treatment of these cells with LPS and 10 μM or 50 μM DJ-X-013, the elongated cells were diminished from the population and the round morphology predominated (FIG. 1E).

To further evaluate the cell morphology under these conditions, cell morphometric analysis using ImageJ software (NIH) was performed. Six morphometric parameters were analyzed, including area, perimeter, major axis, minor axis, aspect ratio, and circularity (FIG. 1F). While the area and perimeter are descriptors of cell size, the major axis, minor axis, aspect ratio, and circularity parameters allowed us to examine the cell shape as either round or elongated. For LPS-treated cells, the cellular area was significantly enlarged in both R and E morphologies relative to that of control cells and those of the LPS (E) morphology had a larger area than cells treated with LPS plus either 10 μM or 50 μM DJ-X-013 (FIG. 1F). Surprisingly, the cells with the LPS (R) morphology exhibited a smaller area than the cells treated with LPS and 10 μM or 50 μM DJ-X-013.

The perimeter and major axis parameters exhibited a similar trend except when comparing cells of the LPS R morphology to cells treated with LPS and 10 μM DJ-X-013. The area, perimeter, and major axis were all highest for LPS (E) cells, although the minor axis of these cells was highly subsided (as expected for the elongated cell morphology). The minor axis increased gradually after treatment with LPS alone or with LPS plus either concentration of DJ-X-013, relative to the minor axis of control cells. The alteration of the major and minor axis was further reflected in the aspect ratio, which was highest for cells of the LPS (E) morphology, while these cells exhibited the lowest circularity. In contrast, there were no significant differences in aspect ratio and circularity when comparing cells of the LPS R morphology to those of cells treated with LPS plus 10 μM or 50 μM DJ-X-013.

Considering that the aspect ratio and circularity values of a circle are both equal to 1, an aspect ratio>1 indicates a higher degree of elongation, while a circularity value<1 indicates a higher degree of protrusions.⁴² Thus, the elevated aspect ratio and lower circularity values of LPS (E) cells emphasize their morphological differences. Furthermore, the distribution of data points for every parameter of LPS (E) cells was very scattered relative to the other cells, suggesting that their morphology was very dynamic. Thus, the results of cellular morphometric analysis after phase contrast microscopy indicate that LPS may induce polarization of cultured RAW264.7 macrophages to an elongated morphology with more protrusions and that the addition of DJ-X-013 after LPS-induction either inhibits the cellular elongation-induced by LPS or reverts the elongated cell morphology to the round morphology despite LPS stimulation.

### Example 5

### DJ-X-013 Attenuates LPS-Stimulated RAW 264.7 Cell Migration

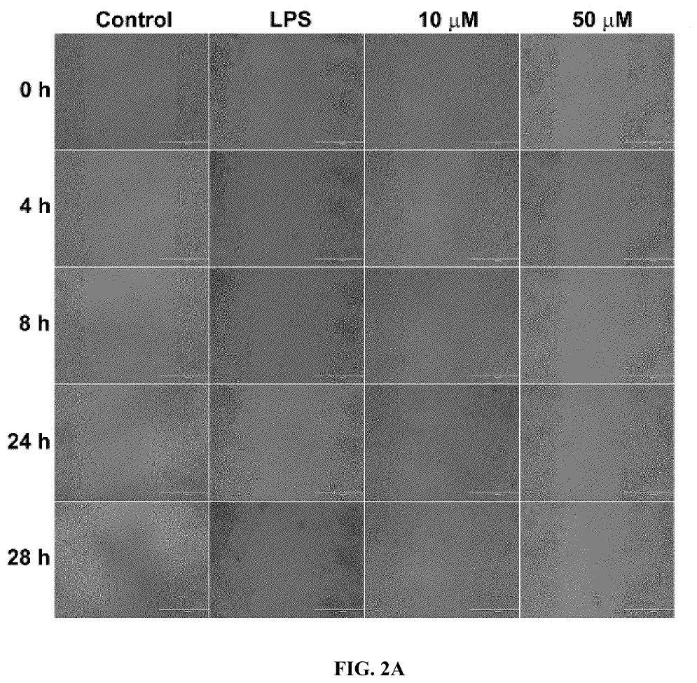

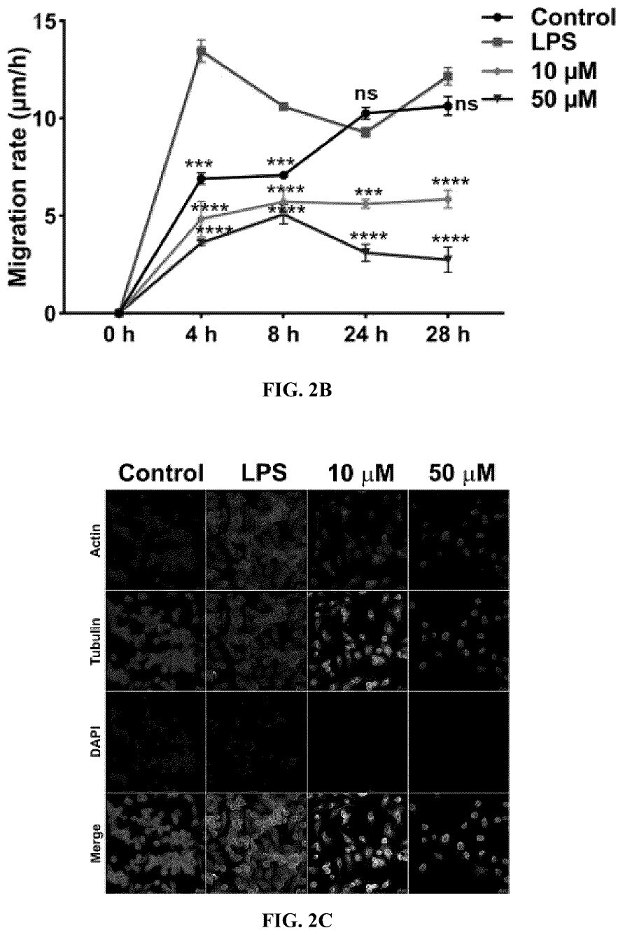

The scratch or wound healing assay monitors the potential of a cell to migrate and was used to calculate the cell migration speed of LPS-stimulated RAW 264.7 cells in the presence or absence of DJ-X-013 by measuring the width of the scratch at different temporal points (FIG. 2A, FIG. 2B). The migration rate was highest after 4 h of LPS treatment, relative to that of the control. In contrast, cells stimulated with LPS and treated with 10 μM or 50 μM DJ-X-013 maintained a slow migration speed from 4 to 28 h, relative to that of control cells or cells treated with LPS. In general, LPS serves to stimulate the migration of macrophages to infected and inflamed tissue. Here, LPS treatment activated RAW264.7 macrophages to migrate to fill the scratch or wound site, whereas this migration was slowed on treatment with DJ-X-013, suggesting that this compound impaired the LPS-induced cell migration.

### Example 6

### DJ-X-013 Treatment of LPS-Stimulated RAW 264.7 Cells Altered the Expression of Cytoskeletal Proteins

Cell morphology and migration are regulated by crosstalk between cytoskeletal proteins, specifically actin filaments, tubulin microtubules, and intermediate filaments. Thus, the effect of 10 or 50 μM DJ-X-013 treatment of LPS-stimulated RAW 264.7 macrophages on actin and tubulin proteins using immunofluorescence (IF) confocal microscopy was examined. In the LPS group, actin became polymerized, while in the LPS+10 μM DJ-X-013 group, tubulin became polymerized, resulting in bright fluorescence signals (FIG. 2C). Cell migration involves dynamic polymerization and depolymerization of actin and tubulin, while inhibition of any of these processes causes defects in cell migration, cell shape, and intracellular transport. Thus, these IF results suggest that DJ-X-013 may modulate actin depolymerization, tubulin polymerization, or both.

### Example 7

### DJ-X-013 Impedes the Expression of Inflammatory Markers in LPS-Stimulated RAW 264.7 Cells

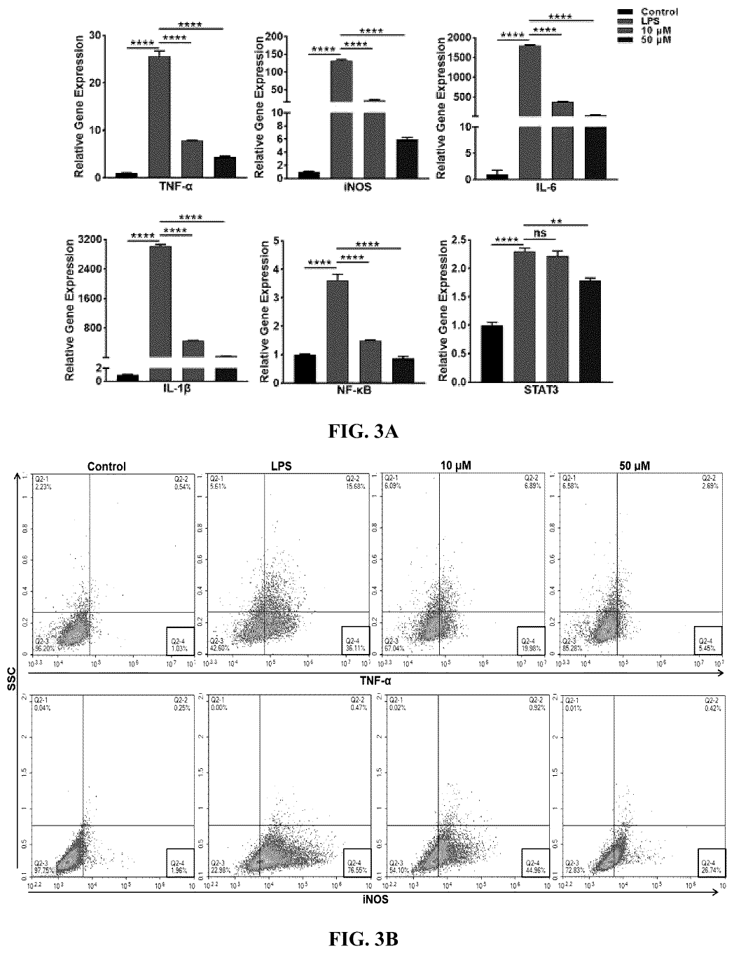

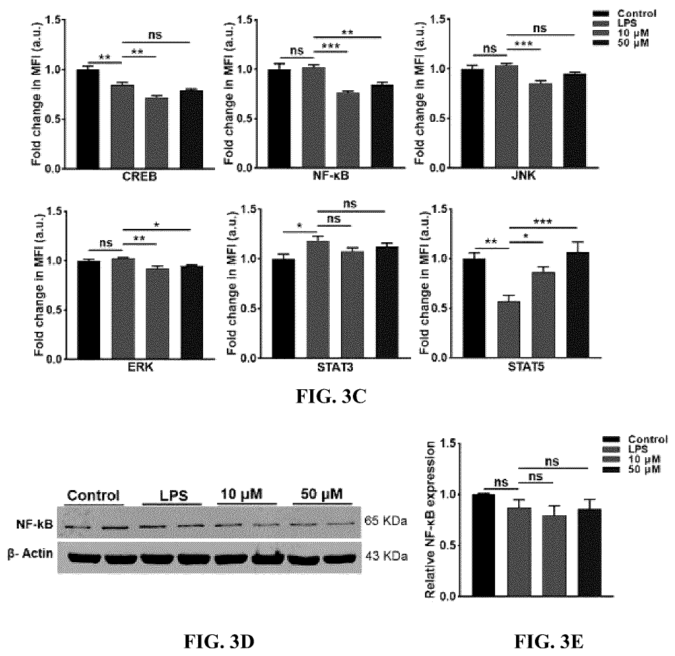

The effect of DJ-X-013 on the expression of inflammatory genes and proteins in LPS-stimulated RAW 264.7 cells was examined (FIG. 3). In RAW 264.7 cells, LPS induces the expression of various inflammatory markers, including TNF-α, inducible nitric oxide synthase (iNOS), interleukin 6 (IL-6), and IL-1β, which in turn stimulate inflammatory signaling pathways such as NF-kB and cJun N-terminal kinase (JNK)/STAT3. The modulatory role of DJ-X-013 on inflammatory marker expression was assessed in LPS-stimulated RAW 264.7 cells using RT-qPCR (FIG. 3A), flow cytometry (FIG. 3B), multiplex assay (FIG. 3C) and immunoblot analysis (FIG. 3D).

LPS treatment of RAW 264.7 stimulated expression of TNF-α, iNOS, IL-6, IL-1β, NF-kB, and STAT3 relative to the control, while treatment with LPS together with 10 μM or 50 μM DJ-X-013 significantly reduced the expression of these genes (FIG. 3A). Interestingly, treatment of the cells with 50 μM DJ-X-013 diminished the expression of STAT3 expression relative to that in the LPS group. Analysis of TNF-α and iNOS expression by flow cytometry showed that DJ-X-013 treatment of LPS-stimulated cells reduced the expression of both proteins (FIG. 3B). Further, the multiplex bead array assay showed that treatment with 10 μM DJ-X-013 strongly decreased the expression of CREB, NF-kB, JNK, and ERK relative to LPS alone (FIG. 3C). Oppositely, DJ-X-013 enhanced STAT5 expression. The expression of p38, p70S6k, and Akt was not altered prominently after LPS and DJ-X-013 treatment (FIG. 10). Finally, when we examined NF-κB expression using an immunoblot assay, we observed a slight decrease in its expression in the presence of LPS showing a diminishing trend (FIG. 3D). Overall, DJ-X-013 effectively reduced the expression of NF-κB, inflammatory markers, and common signaling pathways in RAW 264.7 macrophages after LPS stimulation.

### Example 8

DJ-X-013 Reduces Colon Inflammatory Parameters Associated with Colitis

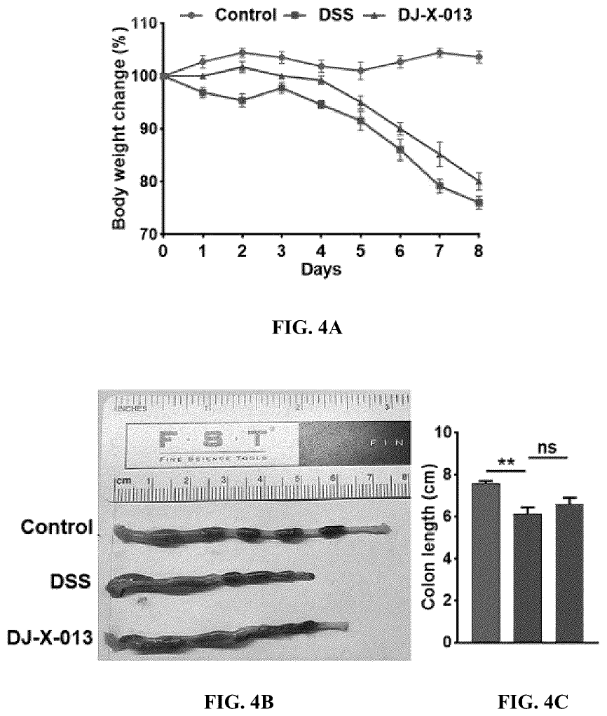

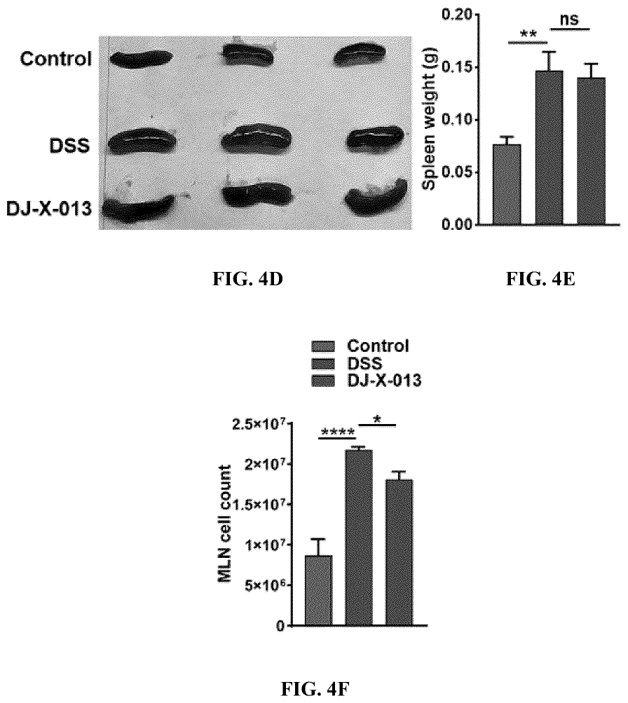

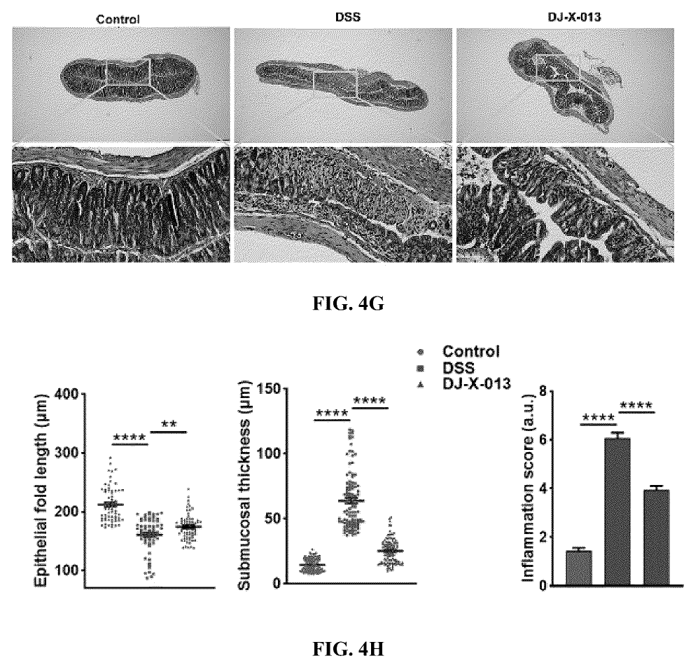

The effect of DJ-X-013 on body weight and colon length in vivo was examined in DSS-induced colitis model in mice. Since no drastic change in body weight for the entire experimental period in naïve mice was observed, the body weight of mice receiving DSS was compared with those receiving DSS+DJ-X-013; the change from the initial body weight was expressed in terms of percentage of body weight change. While the body weight in both groups progressively declined over time, mice that received DSS alone developed colitis, as shown by a more decrease in body weight and mice treated with DSS+DJ-X-013 exhibited an improvement in body weight as compared to DSS group (FIG. 4A). After the experimental endpoint, the mice were euthanized and colons were isolated and measured. The mean colon length of mice with DSS-induced colitis was slightly shorter than that of the colons of DJ-X-013 treated mice, although the difference was not statistically significant (FIG. 4B and FIG. 4C). These data suggest that the administration of DJ-X-013 may have a protective role in maintaining colon length. To obtain a preliminary idea about splenomegaly and inflammation in these mice, the spleen weight and MLNs cells number were determined. The area, volume, and weight of the spleen increased in the DSS group, while spleen weight decreased marginally in mice treated with DSS and DJ-X-013 (FIG. 4D and FIG. 4E). The total number of MLNs cells significantly decreased in the DJ-X-013 group compared to mice treated with DSS alone (FIG. 4F).

Colon pathology was also evaluated, including epithelial fold length, submucosal thickness, and inflammation score (FIG. 4G). The colon pathology in mice with DSS-induced colitis showed loss of colonocyte organization in the epithelium, a hypertrophied submucosal layer, and an increased inflammatory score (FIG. 4G), predominantly due to infiltration by immune cells. In contrast, mice treated with DSS and DJ-X-013 exhibited improved colon histology and a reduced inflammation score relative to the mice treated with DSS alone. The tissue parameters associated with colitis, which include epithelial fold length and submucosal thickness of the colon, were significantly improved following treatment with DJ-X-013 (FIG. 4H). Taken together, these outcomes demonstrate that DJ-X-013 treatment ameliorated colitis in DSS-induced mice by restoring body weight, spleen weight, colon length, and epithelial architecture and reducing inflammatory infiltration.

### Example 9

### DJ-X-013 Treatment Inhibits Infiltration of Neutrophils, TNF-Producing Macrophages, Activated Monocytes, and Neutrophils in the Colon of DSS-Induced Mice

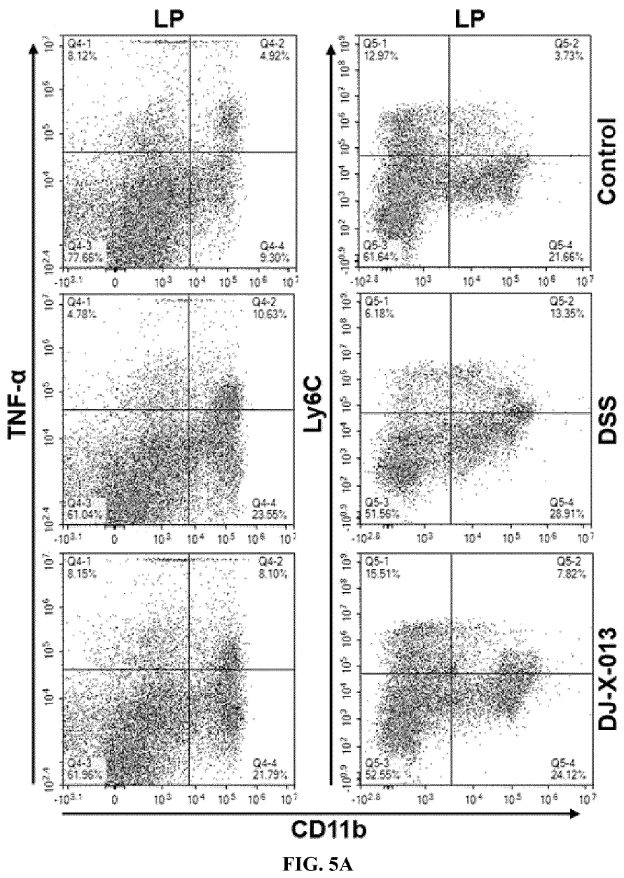

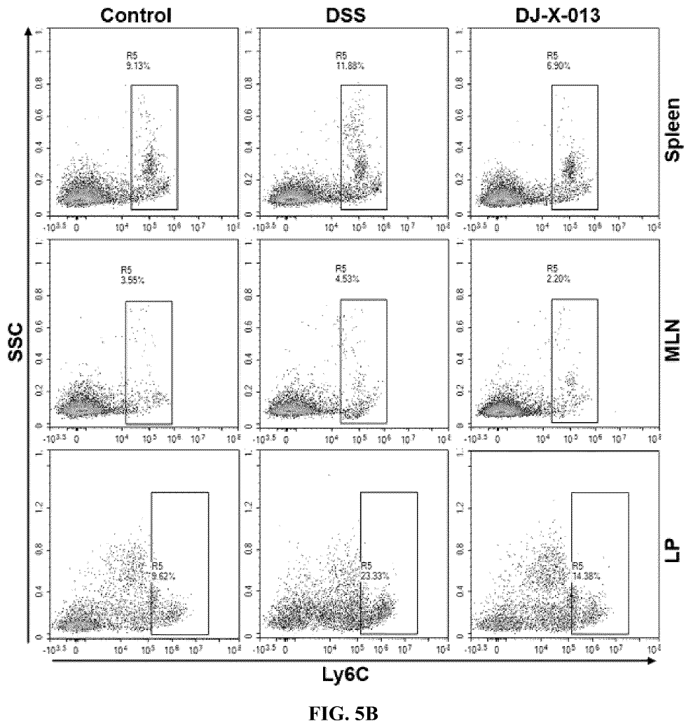

Macrophages express the pro-inflammatory cytokine TNF-α while neutrophils play a role in the development and maintenance of intestinal inflammation and occur in increased numbers during DSS-induced colitis.⁴³ Levels of TNF-α increase in IBD patients and blocking TNF-α production is a crucial aspect of current IBD therapeutics.⁴⁴ Flow cytometry was used to examine the frequency of mucosal TNF-α producing macrophages and systemic neutrophils in DSS-induced colitis in the absence or presence of DJ-X-013. Importantly, a reduction in the frequency of inflammatory cells i.e. CD11b⁺ macrophages producing TNF-α and CD11b⁺Ly6C⁺ inflammatory monocytes in the colon LP after treatment with DSS and DJ-X-013 was observed (FIG. 5A). In mice treated with DSS alone, the percentage of neutrophils increased both in systemic and colon lamina propria (LP), relative to that in control mice (FIG. 5B). In contrast, after treatment of mice with DSS and DJ-X-013, the frequency of neutrophils was reduced in the spleen, MLNs, and colon LP, relative to that observed in mice treated with DSS alone. Taken together, these results demonstrate that treatment with DSS and DJ-X-013 attenuated the number of macrophages, monocytes, and neutrophils in the mouse colon and that DJ-X-013 may be effective in reducing colitis.

### Example 10

### DJ-X-013 Decreases Mucosal-Activated T Cells and Revises NK/NKT Cells

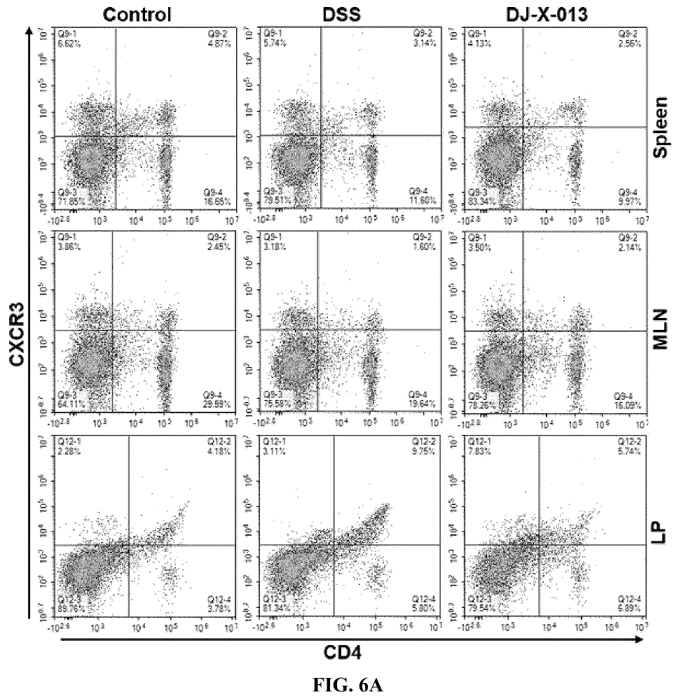

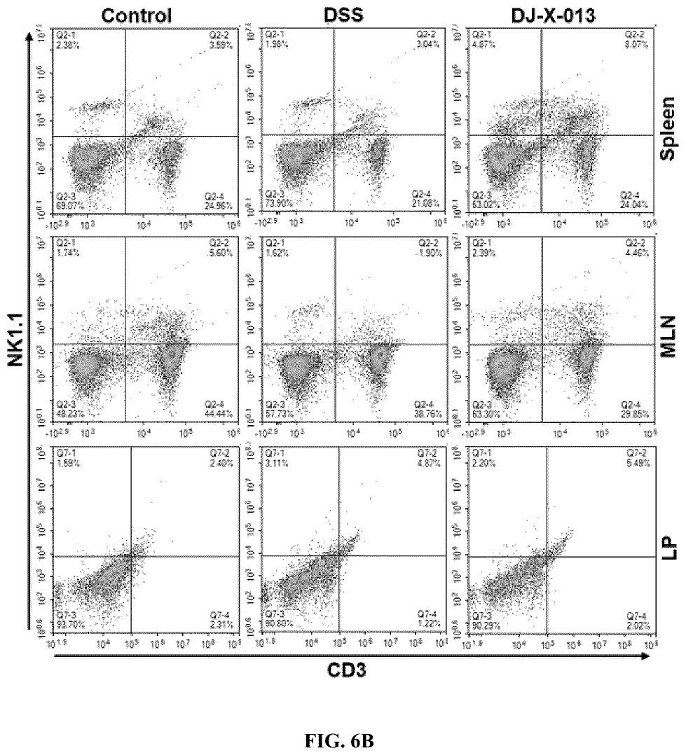

That inflammation in the mouse colon is primarily mediated by CD4⁺ T helper 1 lymphocytes (Th1 cells) was previously demonstrated.^{45,46} Natural killer (NK) cells and natural killer T-cells (NKT cells) participate in the regulation of the intestinal immune response,⁴⁷ and in differentiating naive CD4⁺ T cells into Th1 cells.⁴⁸ Flow cytometry analysis was used to explore the effect of DJ-X-013 treatment on populations of activated CD4⁺ T cells isolated from spleen, MLN, and colon LP of DSS+DJ-X-013 treated mice (FIG. 6A). Whereas the frequency of activated T cells (CD4⁺CXCR3⁺) in the spleens and MLNs was not much altered after DJ-X-013 treatment, the percentage of activated T cells in the colon LP declined in the DJ-X-013 group relative to the DSS alone group (FIG. 6A, upper right quadrants). These findings suggest that treatment of mice with DJ-X-013 markedly reduces the frequency of CD4⁺CXCR3⁺ T cells in the colon LP, which may protect them against DSS-induced colitis.

The frequency of mucosal and systemic NK and NKT in mice treated with DSS in the absence or presence of DJ-X-013 treatment was also evaluated. The induction of colitis in the mice with DSS slightly decreased the percentage of NK (FIG. 6B, upper left quadrants) and NKT (FIG. 6B, upper right quadrants) cells in spleen and MLN, relative to the control mice. Treatment with DJ-X-013 increased the percentages of NK and NKT cells in the spleens and MLNs relative to DSS alone (FIG. 6B). However, in the colon LP, both NK and NKT cell populations increased in mice treated with DSS alone, relative to the control (FIG. 6B, upper left and right quadrants), while treatment with DSS+DJ-X-013 was associated with a decreased percentage of NK cells and elevated NKT cell infiltration in colon LP, relative to the percentages observed in mice treated with DSS alone. Taken together, results demonstrate that treatment with DJ-X-013 differentially modulates both NK and NKT cells in systemic and mucosal organs relative to DSS.

### Example 11

DJ-X-013 Induces Infiltration of the Colon with MDSCs and DCs

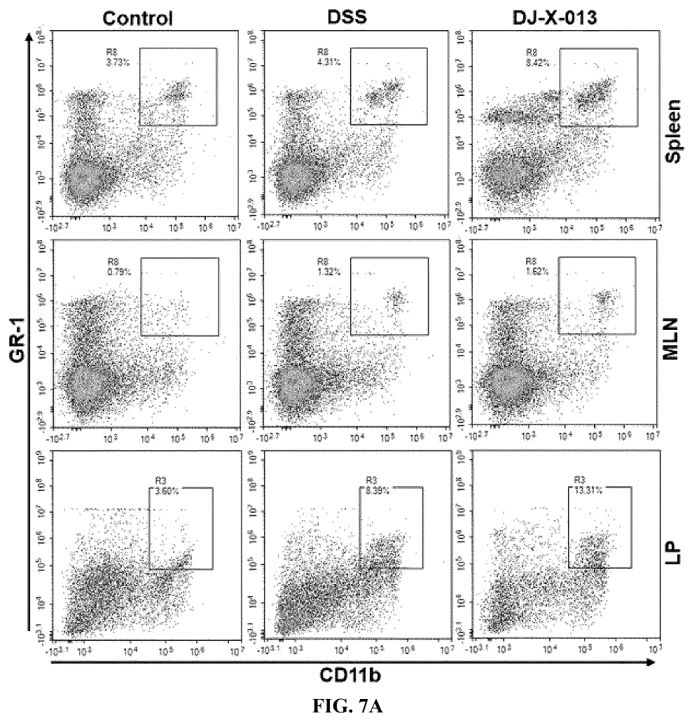

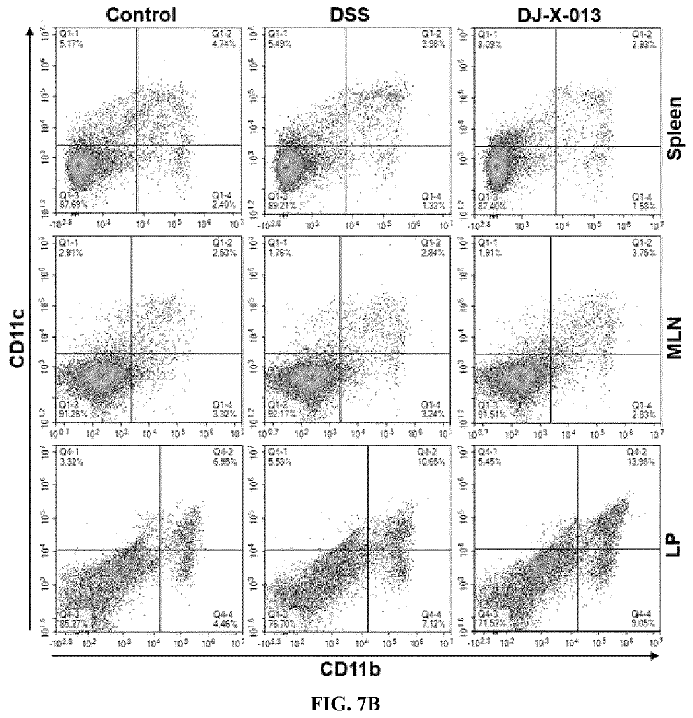

MDSCs are a heterogeneous population of immature myeloid cells that serve as crucial players in the prevention and treatment of human diseases involving chronic inflammation.³⁶ T cells become activated during colitis as the result of efficient uptake and presentation of antigens to T-cells by dendritic cells (DCs).⁴⁹ The effect of DJ-X-013 treatment on the frequency of systemic mucosal MDSCs (CD11b⁺GR-1⁺) and DCs (CD11b⁺CD11c⁺) was assessed by flow cytometry. On induction of colitis in mice with DSS, the frequency of MDSCs was slightly increased in the spleens, MLNs, and colon LP, relative to those of control mice (boxed cells in FIG. 7A). Interestingly, treating mice with DSS and DJ-X-013 led to a prominently increased frequency of MDSCs in spleens, MLNs, and colon LP relative to those after treatment with DSS alone (FIG. 7A in boxed cells). These results suggest that DJ-X-013 treatment induced the frequency of MDSCs in the spleen, MLNs, and LP, thereby protecting the colon from colitis.

The frequency of DCs frequency in control mice and those treated with DSS or DSS+DJ-X-013 was evaluated. Differential changes in the frequency of systemic splenic DC and mucosal MLNs, and LP in mice treated with DSS and DJ-X-013 relative to those treated with DSS alone were observed. Notably, the frequency of the splenic DCs decreased in mice treated with DJ-X-013 vs DSS alone (FIG. 7B, upper right quadrants). In contrast, the numbers of DCs in the MLNs and colon LP significantly increased on treatment with DSS and DJ-X-013 relative to those detected on treatment with DSS alone (FIG. 7B, right quadrants). Taken together, these results propose that the differential changes in the frequency of DCs that occur at both systemic and mucosal sites during colitis might result from an effector function in the colon to suppress colitis.

### Example 12

### DJ-X-013 Suppresses Inflammatory Th17 Cell Frequency During Colitis

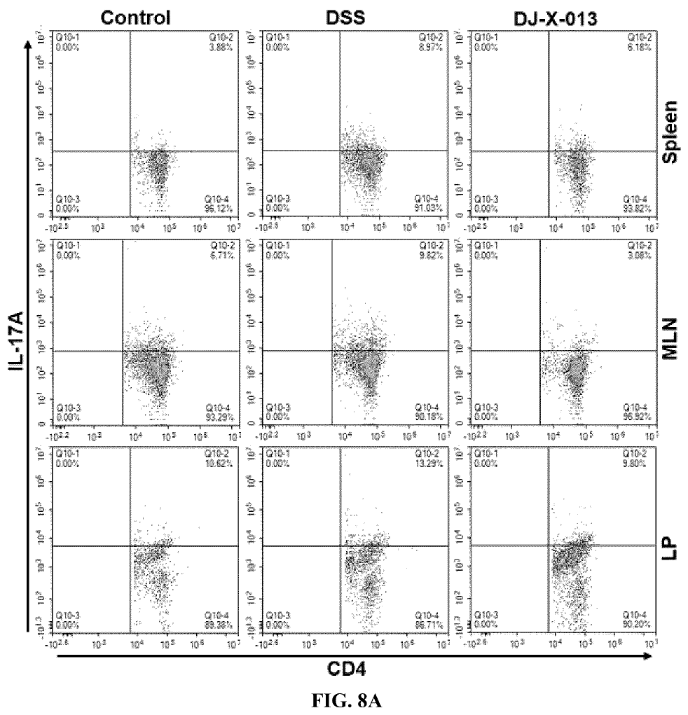

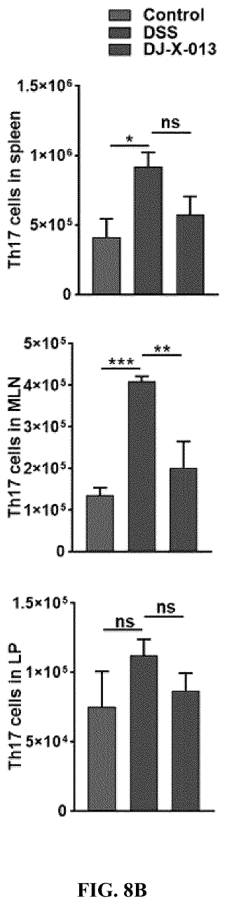

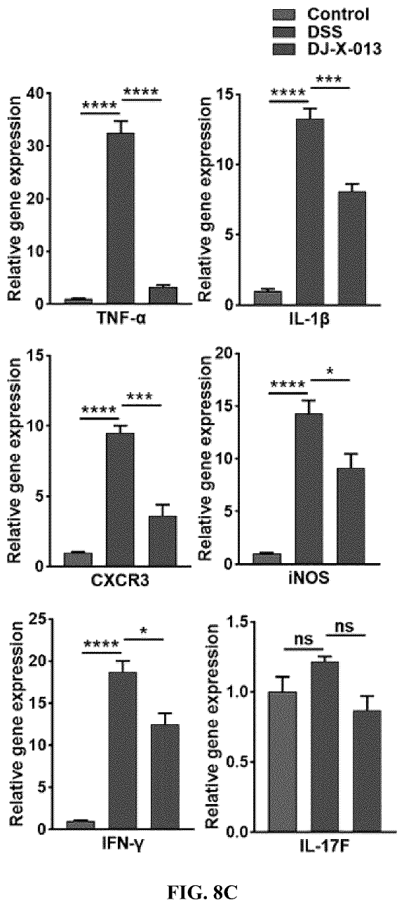

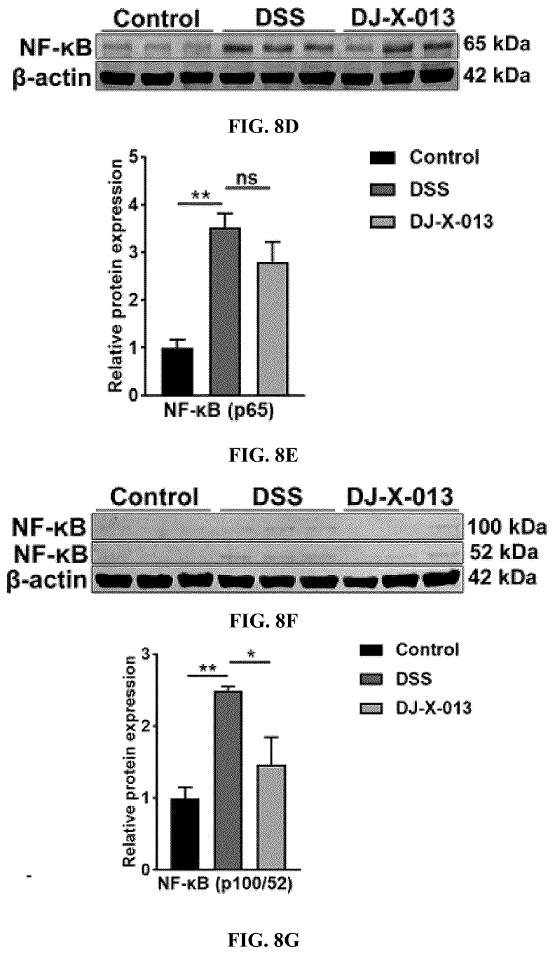

In both CD patients and experimental animal models of colitis, both the frequency of Th17 cells and the expression of IL-17 increase.^{25,50} Since the role of IL-17 in intestinal inflammation remains controversial, whether Th17 cells play any role in DSS-induced colitis and whether they would be affected by DJ-X-013 treatment was examined. Flow cytometry was used to show that the frequency of both systemic and mucosal CD4⁺ Th17 cells decreased after treatment of mice with DSS and DJ-X-013 relative to mice treated with DSS alone (FIG. 8A, upper right quadrant). The number of Th17 cells increased in mice tested with DSS alone but decreased significantly after treatment with DSS and DJ-X-013 (FIG. 8B). Thus, DJ-X-013 treatment appeared to modify the frequency and number of Th17 cells in mice with DSS-induced colitis, suggesting a inhibitory role on the Th17 cell response in DSS-induced colitis.

### Example 13

### DJ-X-013 Restricts the Inflammatory Response by Targeting the NF-κB Pathway in DSS-Induced Model of Colitis in Mice

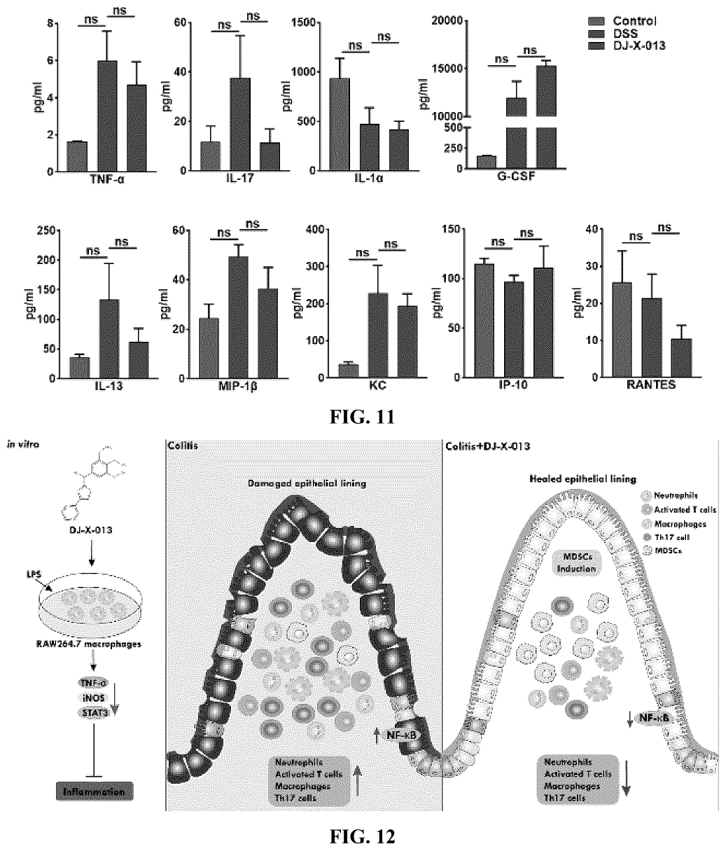

IBD patients exhibit high levels of activated NF-κB,⁵¹ which regulates many inflammatory markers.⁵² Therefore, the gene and protein expression of inflammatory mediators in colon tissue after treatment of mice with DSS and DJ-X-013 was measured using RT-qPCR and immunoblot analysis. The expression of mRNA encoding pro-inflammatory markers TNF-α, IL-1β, CXCR3, iNOS, and IFN-γ as well as IL-17F in the colon was significantly reduced after mice were treated with DSS and DJ-X-013, relative to DSS alone (FIG. 8C). Measurement of the accumulation of canonical and non-canonical NF-κB protein in colon tissue by immunoblot observed a reduction in NF-κB protein expression in the treatment of the mice with DSS and DJ-X-013, relative to DSS alone (FIG. 8D, FIG. 8F). Treatment of mice with DSS and DJ-X-013 suppressed the expression of systemic inflammatory cytokines and chemokines relative to the levels observed on treatment with DSS alone (FIG. 11). Thus, taken together, compound DJ-X-013 exerts its anti-inflammatory action, at least in part, by repressing expression of the transcription factor NF-kB.

### Example 14

### Synthesis of (4-phenylthiazol-2-yl) (3,4,5-trihydroxyphenyl) methanone (DJ-X-025)

DJ-X-025 was synthesized in three steps, as described below and shown in FIG. 13D. These three steps are described below.

### Synthesis of (4-Phenylthiazol-2-yl) (3,4,5-trimethoxyphenyl) methanol (compound 3)

First, to a solution of 4-phenylthiazole-2-carbaldehyde (compound 1; 0.568 g, 3 mmol) in 20 mL THF was slowly added a THF solution of 3,4,5-trimethoxyphenylmagnesiumbromide (compound 2) 0.5 N, 6.0 mL, 3 mmol) at 0° C. under an argon atmosphere. The mixture was allowed to stir for 2 h until the starting aldehyde (compound 1) disappeared. The reaction was then quenched with saturated NH₄Cl, extracted with ethyl ether, and dried with anhydrous MgSO₄. The solvent was removed under reduced pressure to yield a crude product, which was purified by column chromatography to obtain a pure target compound (compound 3). Yield 72.9%. Purity (LC, tₘᵢₙ=3.59) 99.70%. UV λₘₐₓ 254.45 nm, MS (ESI) m/z 358.10 [M+H]⁺; LCMS (ESI) m/z calculated for C₁₉H₁₉NO₄S 358.1113 [M+H]⁺; found: 358.1104 [M+H]⁺; ¹H NMR (CDCl₃, 400 MHz) δ 7.89 (d, J=7.2 Hz, 2H, ArH), 7.45 (d, J=7.2 Hz, 2H, ArH), 7.42 (s, 1H, ArH), 7.34 (t, J=7.6 Hz, 1H, ArH), 6.76 (s, 2H, ArH), 6.03 (s, 1H, CH—OH), 3.86 (s, 6H, (OCH₃) 2), 3.85 (s, 3H, OCH₃), 1.68 (bs, 1H, OH); ¹³C NMR (CDCl₃, 100 MHz) δ 173.55, 154.92, 153.46 (2C), 137.91, 136.79, 134.10, 128.80 (2C), 128.30, 126.32 (2C), 103.51 (2C), 73.92, 60.87, 56.15 (2C).

### Synthesis of (4-Phenylthiazol-2-yl) (3,4,5-trimethoxyphenyl) methanone (Compound 4)

Next, a solution of hydroxy compound 3 (343 mg, 0.96 mmol) in 30 mL anhydrous CH₂Cl₂ (DCM) was added to Dess-Martin periodinane (814 g, 1.92 mmol). The mixture was allowed to stir for 3 h at room temperature (RT) until starting compound 3 disappeared. The solution was quenched with saturated Na₂S₂O₃ solution, extracted with DCM, and dried with anhydrous MgSO₄. The solvent was removed under reduced pressure to yield a crude product that was purified by column chromatography to give a white compound as a designed compound (compound 4). Yield 60.1%. UV λₘₐₓ 251.45 nm. MS (ESI) m/z 356.07 [M+H]⁺. Purity (LC, tₘᵢₙ=4.30) 97.35%. LCMS (ESI) m/z calculated for C₁₉H₁₇NO₄S 356.0957 [M+H]⁺; found: 356.0955 [M+H]⁺. ¹H NMR (CDCl₃, 400 MHz) δ 8.11 (s, 1H), 7.99 (m, 2H), 7.48 (m, 3H), 7.26 (s, 2H), 3.99 (s, 3H), 3.97 (s, 6H). ¹³C NMR (CDCl₃, 100 MHz) δ 181.91, 167.86, 157.32, 152.80, 143.21, 133.86, 129.78, 128.83 (2C), 128.78, 126.54, 126.44 (2C), 119.89, 108.92, 61.60, 108.92 (2C).

### Synthesis of (4-Phenylthiazol-2-yl) (3,4,5-trihydroxyphenyl) methanone (DJ-X-025)

A mixture of (4-phenylthiazol-2-yl) (3,4,5-trimethoxyphenyl) methanone (compound 4, 276 mg, 0.78 mmol) in dry DCM (20 mL) at 0° C. under argon gas was treated dropwise with BBr3 (1M in DCM, 3.1 mL, 3.1 mmol). The resulting mixture was allowed to warm to room temperature and stirred overnight, after which time it was added dropwise to a stirring mixture of ice water (20 mL). The mixture was stirred for 30 min at room temperature, then filtered and dried with anhydrous Na₂SO₄ to provide the product (DX-J-025) as a yellow solid. Yield 72%. MS (ESI) m/z 314.12 [M+H]⁺, 312.15 [M−H]⁻. Purity (LC, tₘᵢₙ=3.26) 97.93%. LCMS (ESI) m/z calculated for C₁₆H₁₁NO₄S 314.0487 [M+H]⁺; found: 314.0486 [M+H]⁺. ¹H NMR (Acetone-d₆, 400 MHz) δ 8.89 (bs, 3H, OH), 8.33 (s, 1H), 8.11 (d, J=8.0 Hz, 2H), 7.98 (s, 2H), 7.50 (t, J=8.0 Hz, 2H), 7.43 (t, J=8.0 Hz, 1H). ¹³C NMR (Acetone-d₆, 100 MHz) δ 181.19 (CO), 171.55, 168.44, 156.99, 145.20, 139.68, 134.08, 129.22, 128.91 (2C), 128.56, 126.43, 125.87 (2C), 120.19, 111.25.

### Example 15

### DJ-X-025 Predicted to Display High Gastrointestinal (GI) Absorption and not Permeable to the Blood-Brain Barrier (BBB)

In this example, the constituents of thiazole connected to galloyl analogue as depicted in (FIG. 13A, FIG. 21) were examined. The molecule demonstrated beneficial anti-inflammatory activity and provided a computer-aided prediction as a drug option as the following. DJ-X-025 was evaluated through computational methods known as SwissADME programs (www.swissadme.ch, Molecular Modeling Group-Swiss Institute of Bioinformatics, Lausanne, CH). The synthesized DJ-X-025 calculated and expected physical properties, such as ADME (Absorption, Distribution, Metabolism, and Excretion) and other drug types to use the pharmaceutical aids as shown in (FIG. 13B and Table 4). In FIG. 13B, the round yellow yolk-like sphere represents the BBB (Blood-Brain Barrier) permeation region, and the oval white part represents the HIA (Human Intestinal Absorption) region. The grey region represents the low absorption and limited brain permeation region. The right-hand side box provides the option to show the molecule DJ-X-025, legends of the BOILED-Egg model, and remarks. The query of DJ-X-025 is visualized as a red hollow sphere located in the white part (HIA). The red color represents PGP+ (P-glycoprotein positive) based on its property located in the white region, resulting also in no penetration in BBB and optimized performance in physical properties.

TABLE 4
Predicted physicochemical, pharmacokinetic,
and drug-likeness properties of DJ-X-025.

| | Physicochemical parameters | | | | | | |
|---|---|---|---|---|---|---|---|
| | | MW | | | | | |
| DJ-X-025 | Formula | (g/mol) | HA | RB | HBA | HBD | FC |
| | C₁₆H₁₁NO₄S | 313.33 | 22 | 3 | 5 | 3 | 0 |

| | Pharmacokinetic parameters | | |
|---|---|---|---|
| Structure | MLOGP | GI absorption | BBB permeation |
| | 0.5 | High | No |

| 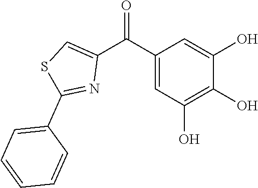 [SMILES: O=C(c1cc(O)c(O)c(O)c1)c1csc(-c2ccccc2)n1] | Drug-likeness properties | |
|---|---|---|
| | Lipinski's Rules of Five | Bioavailability |
| | Yes: 0 violation | 0.55 |

Properties were determined using the SwissADME web tool. 43å
Abbreviations used: HA, Number of heavy atoms; RB, Number of rotatable bonds; HBA, Number
of H-bond acceptors; HBD, Number of H-bond donors; FC, Fraction Csp3; MLOGP, LogPo/w
Topological method; GI, Gastrointestinal; BBB, Blood-brain barrier.

DJ-X-025 is the Bioavailability Radar calculated in several physical properties as marked as LIPO (Lipophilicity), SIZE, POLAR (Polarity), INSOLU (Insolubility), INSATU (Insaturation; 0.25<Fraction Csp3<1), FLEX as shown in (FIG. 13C). Table 4 shows the key factors of physical properties for drug-likeness such as physicochemical, pharmaceutical, and drug-likeness properties of the galloyl DJ-X-025, especially high GI absorption and no penetration on BBB with high bioavailability matched with Lipinski's Rules of Five.

The physical properties of the molecule DJ-X-025 ((4-Phenylthiazol-2-yl) (3,4,5-trihydroxyphenyl) methanone), which is comprised of a thiazole scaffold substituted at the 2 positions with a galloyl group, are shown in (FIG. 1A). DJ-X-025 is believed to demonstrate anti-inflammatory activity similar to that of other thiazole derivatives. Thus, the properties of DJ-X-025 were examined (FIG. 13B and Table 4), using the SwissADME prediction package (www.swissadme.ch, Molecular Modeling Group-Swiss Institute of Bioinformatics, Lausanne, CH) and a similar model to depict a molecule's predicted BBB permeation of DJ-X-025 (FIG. 13B). DJ-X-025 (FIG. 13B, red circle) is predicted to have no BBB penetration and, thus, DJ-X-025 would be considered to have improved physical properties for use in the gut. DJ-X-025 physicochemical properties are determined and are depicted as a red line (FIG. 13C). The oral bioavailability properties predicted are depicted in (FIG. 13C).

DJ-X-025 physicochemical, pharmaceutical, and drug-likeness properties are depicted in Table 4. Lipinski's rule of five was used to predict the oral bioavailability of DJ-X-025. DJ-X-025 was considered to be bioavailable as shown in (Table 4). Taken together, these results suggest that this molecule is likely to be highly drug-like, with high gastrointestinal (GI) absorption, no penetration of the BBB, and high bioavailability.

### Example 16

### DJ-X-025 Did not Induce Notable Change in the Viability of RAW264.7 Macrophages

DJ-X-025 in vitro effect on the viability, apoptosis, and inflammatory response on cultured mouse RAW264.7 macrophages. After the synthesis and purification of DJ-X-025, the effect of DJ-X-025 was tested on cellular apoptosis on RAW264.7 macrophage cells. RAW264.7 macrophages were stimulated with LPS and treated them for 24 h with various concentrations of DJ-X-025 (5, 10, 20, and 50 μM) and performed fluorescein isothiocyanate-labeled Annexin V and PI apoptosis assay. DJ-X-025 (5, 10, 20, and 50 μM) induced 2.3±0.28%, 2.95±0.27%, 7.65±0.63%, and 12.55±1.36% of apoptosis respectively in the LPS stimulated RAW264.7 macrophage cell population (FIG. 13E). Therefore, >90% of the cells were viable when cells were treated with DJ-X-025 in the 5 to 20 μM concentration range for 24 h. However, >85% of cells remained alive after 24 h at 50 μM DJ-X-025 treatment (FIG. 13E). Thus, DJ-X-025 is safe at 5 to 20 μM concentration range. Based on the results of the apoptosis assay, concentrations of were selected 5 μM, 10 μM, and 20 μM for all in vitro experiments.

### Example 17

### DJ-X-025 Treatment Restricted Morphological Variation of LPS-Stimulated RAW 264.7 Macrophages and Altered MC38 Cell Morphology

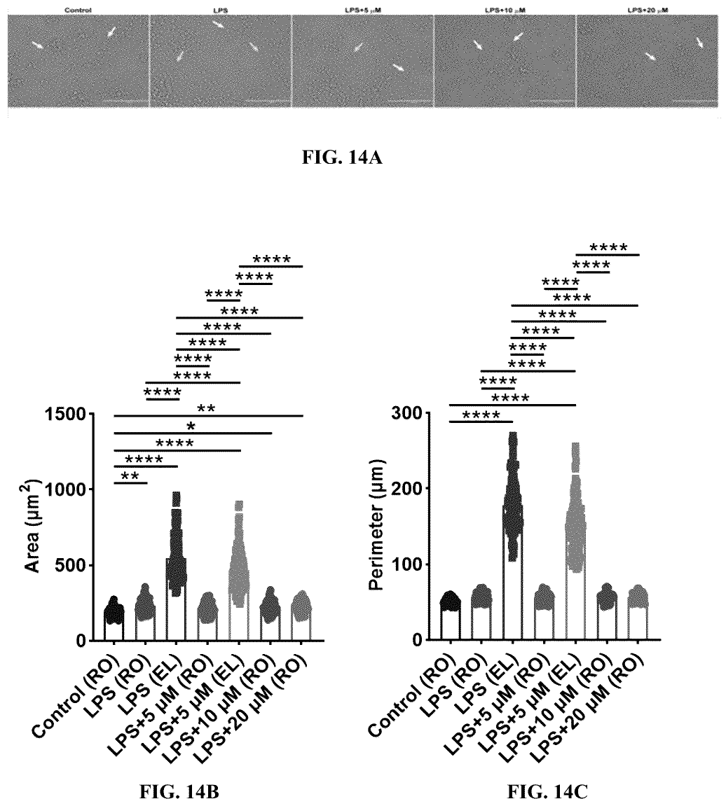

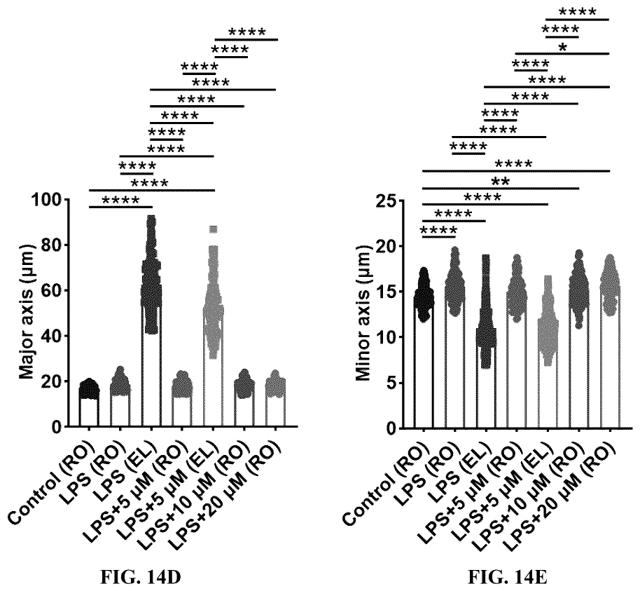

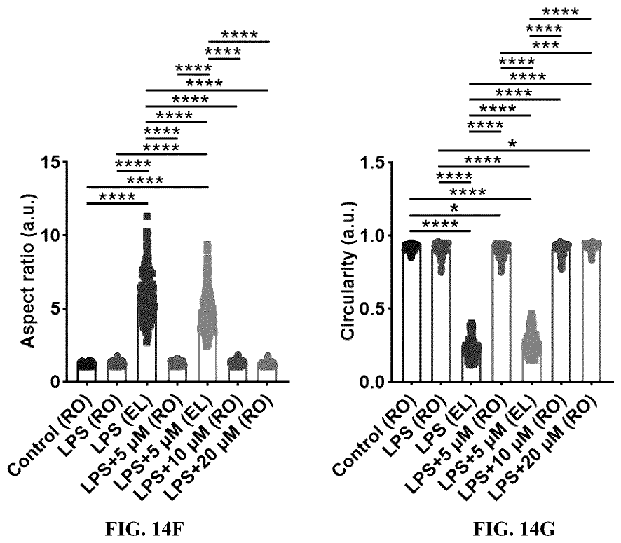

The morphologies of RAW 264.7 macrophages were examined in the control, LPS, and LPS+DJ-X-025 intervention groups using phase contrast microscopy. Untreated control cells exhibited a morphology that was mostly round, as indicated by white arrows (FIG. 14A, Control). However, on stimulation with LPS, RAW 264.7 macrophages exhibited two distinct morphologies: round (RO) cells that resembled the control cells (FIG. 2A, LPS, white arrows) and elongated (EL) cells (FIG. 14A, LPS, yellow arrows). When LPS-stimulated RAW 264.7 macrophages were treated with 5, 10, or 20 μM DJ-X-025, the cells with the EL morphology gradually disappeared from the culture in a dose-dependent manner and those with the RO morphology became predominant as in control cells (FIG. 14A, white arrows).

These morphological changes were examined in more detail using ImageJ software (NIH). Two morphometric parameters of cell size were analyzed-area and perimeter and four parameters of cell shape (RO or EL), specifically major axis length, minor axis length, aspect ratio, and circularity (FIG. 14B through FIG. 14G). LPS-treated cells of both phenotypes (RO and EL) had areas that were significantly larger than those of control cells (FIG. 14B). LPS-treated cells of the EL morphology had a larger area than that of cells treated with LPS plus 5, 10, or 20 μM DJ-X-025, but LPS-induced cells of the RO morphology were similar in area to those treated with these dosages of DJ-X-025 (FIG. 14B). RO cells of all groups exhibited similar perimeter and major axis length (FIG. 14C and FIG. 14D). Cells with EL morphologies treated with LPS alone or together with 5 μM DJ-X-025 differed markedly in all six phenotypic characteristics from cells with RO morphologies of all groups (FIG. 14B through FIG. 14G).

Aspect ratio and circularity contribute to cell shape. Both the aspect ratio and circularity value of a circle are 1. A cell with an aspect ratio>1 would exhibit a higher magnitude of elongation, while a cell with a circularity value<1 would show a greater degree of protrusion (cellular filopodia formation). Thus, the higher aspect ratio and minimal circularity value of the cells with the EL morphologies (LPS and LPS+5 μM DJ-X-025) are consistent with their morphological differences with RO cells in the other groups. Compared to LPS-treated cells of the EL morphology, EL cells treated with LPS and 5 μM DJ-X-025 were notably reduced in area, perimeter, major axis, minor axis, and aspect ratio parameters and increased in circularity (FIG. 14B through FIG. 14G). These results demonstrate that DJ-X-025 exerted its most striking effects on cells with elongated morphologies. Interestingly, the highly scattered distribution of the individual data points for elongated cells (LPS alone or with 5 μM DJ-X-025) in every parameter demonstrated that their morphologies were dynamic. These results indicate that LPS induced polarization of RAW264.7 macrophages to an elongated morphology with more protrusions or filopodia, while treatment with DJ-X-025 either restricts cellular elongation or converts cells of the elongated morphology to round cells

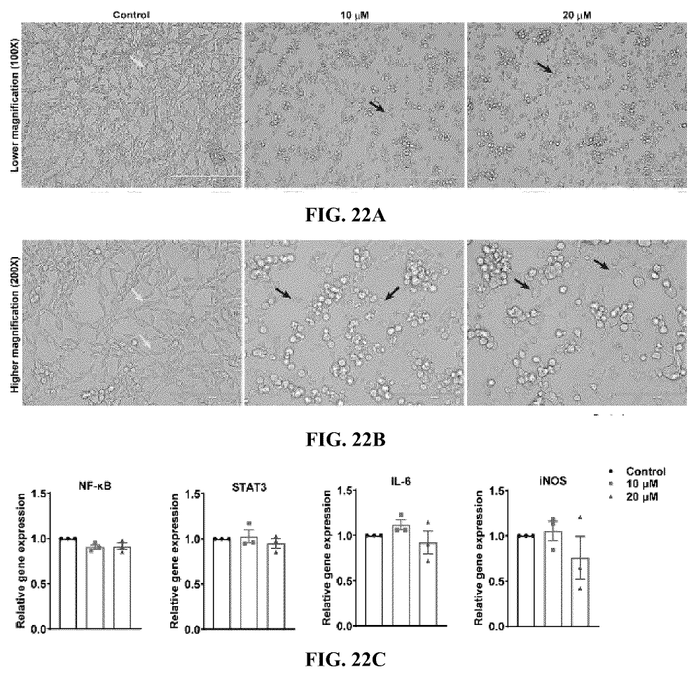

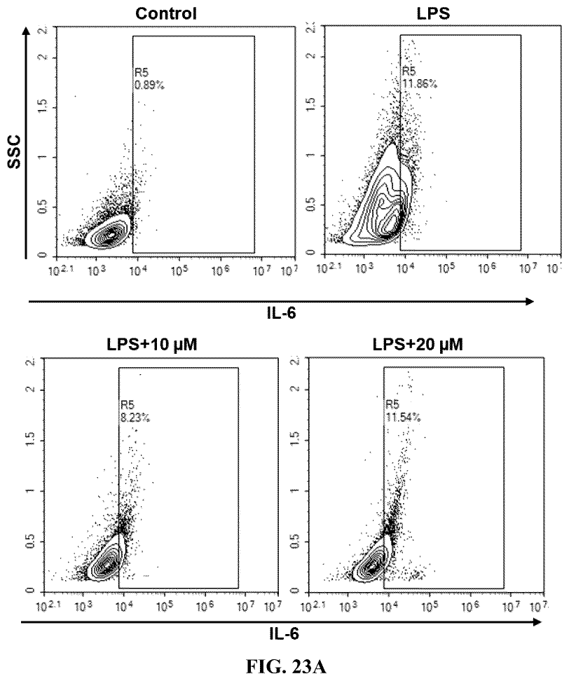

The alteration of cell morphology was also tested in MC38 mouse colon adenocarcinoma cells by treating the cells with 10 μM and 20 μM DJ-X-025 for 24 h. MC38 cells showed elongated spindle-shaped morphology in untreated (control) normal growth conditions, (FIG. 22A, FIG. 22B). However, elongated cell morphology drastically altered towards a round shape in 10 μM and 20 μM DJ-X-025 treated groups (FIG. 22A, FIG. 22B). These results further indicate that DJ-X-025 mediates cell shape by alterations in cytoskeletal proteins.

### Example 18

### DJ-X-025 Treatment Altered the Localization of Cytoskeletal Proteins in LPS-Stimulated RAW 264.7 Macrophages

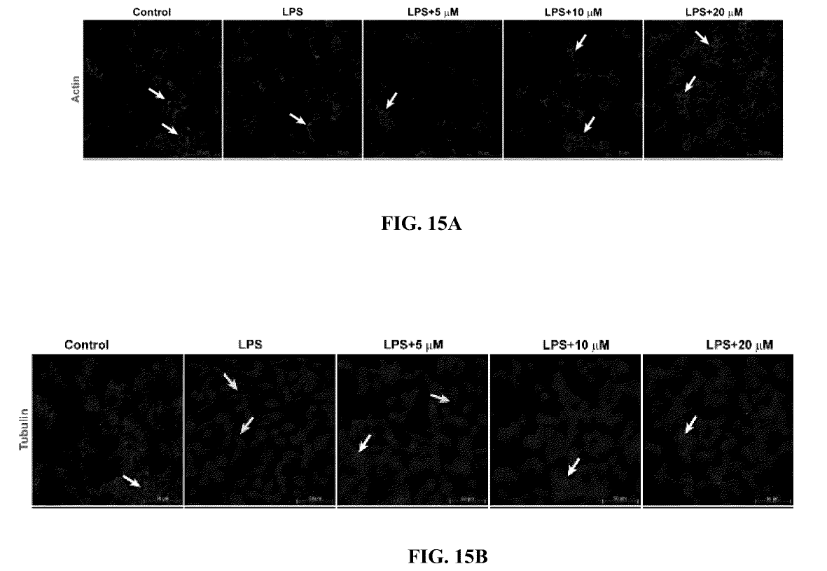

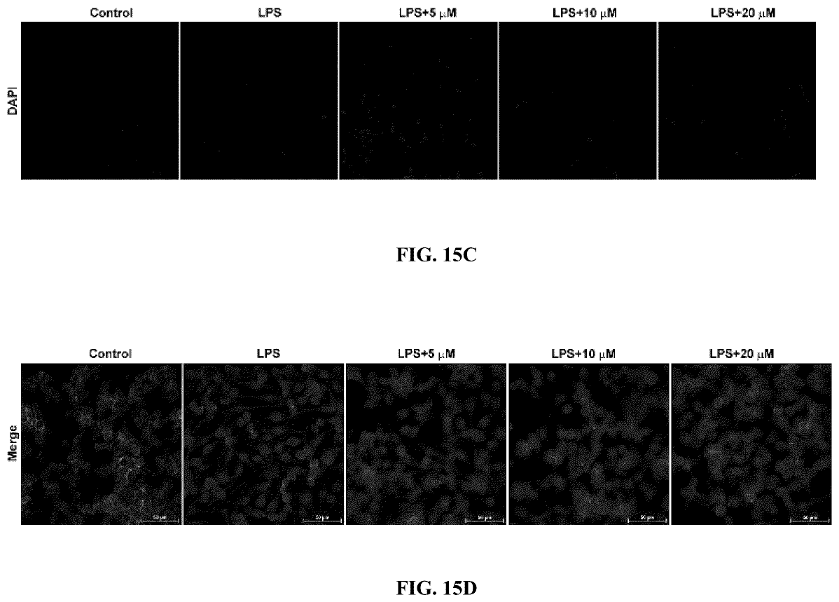

As maintenance of cellular morphology involves crosstalk between cytoskeletal proteins actin and tubulin, the impact of treating LPS-stimulated RAW 264.7 macrophages with DJ-X-025 on actin and tubulin using immunofluorescence (IF) confocal microscopy was explored (FIGS. 15A-3D). In untreated control cells, actin and tubulin were highly concentrated in the cortical region of the cellular periphery (FIG. 15A and FIG. 15B, white arrows) and sparsely distributed in the cytoplasm. In elongated cells treated with LPS alone or with 5 μM DJ-X-025, tubulin was distributed in the cytoplasm, with less localization in the cell peripheral region (FIG. 15B, yellow arrows). However, there was no prominent change in the nucleus visible on staining with DAPI in any of the groups (FIG. 15C). As the concentration of DJ-X-025 increased, actin and tubulin gradually became more prominent on the cell periphery in a dose-dependent manner, which can be best visualized in the merged panel (FIG. 15D). Thus, these results demonstrate that DJ-X-025 may alter cellular morphologies by modulating the distribution and localization of cytoskeletal proteins.

### Example 19

### DJ-X-025 Reduced the Expression of Inflammatory Marker Transcripts and Proteins in LPS-Stimulated RAW 264.7 Macrophages and Slightly Diminished in MC38 Cells

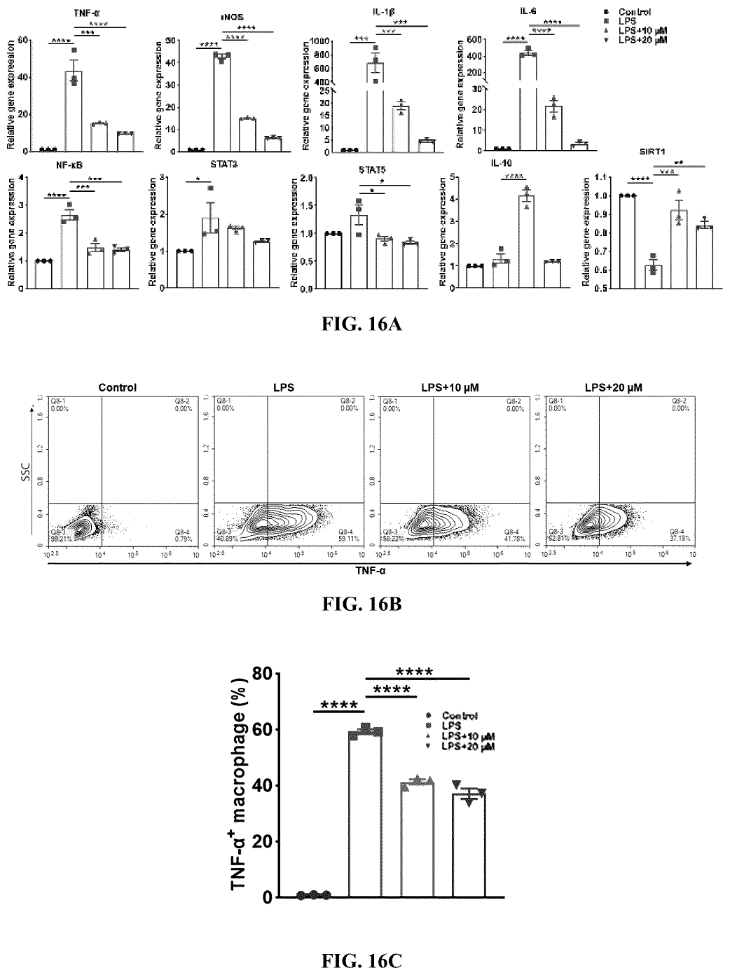

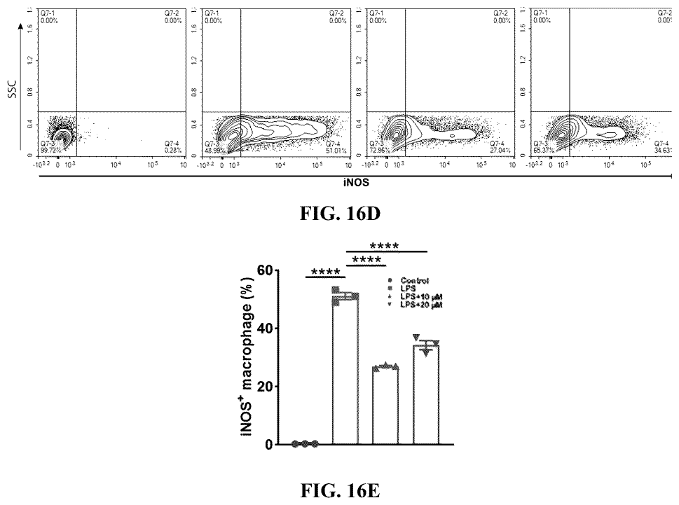

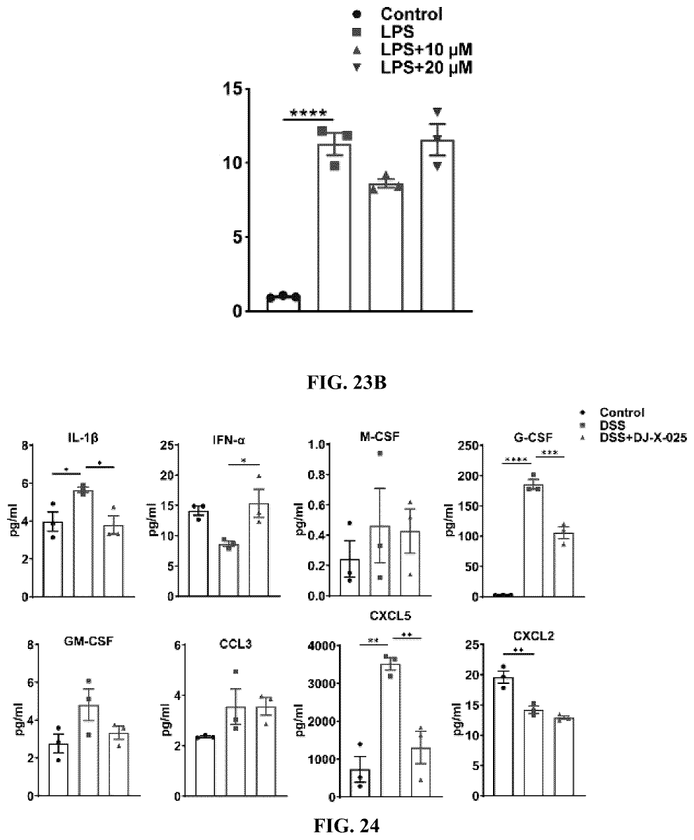

Due to the more prominent effects of LPS plus the higher dosages (10 μM and 20 μM) of DJ-X-025 on morphology and cytoskeleton proteins relative to LPS alone, the LPS+10 μM and LPS+20 μM groups along with control and LPS groups were used to evaluate the anti-inflammatory properties of DJ-X-025 using RT-qPCR (FIG. 16A) and flow cytometry analysis (FIG. 16B and FIG. 16D). Whereas LPS induced the expression of inflammatory markers, including TNF-α, iNOS, IL-1β, IL-6, NF-κB, STAT3, and STAT5 relative to the untreated control, DJ-X-025 notably inhibited the expression of these inflammatory genes relative to LPS alone (FIG. 16A). In contrast the reduced expression of anti-inflammatory markers IL-10 and SIRT1 by LPS induction was significantly induced by the DJ-X-025 treatment (FIG. 16A). DJ-X-025 also leads to a reduction of STAT3, IL-6, and iNOS in MC38 cells especially 20 μM concentration (FIG. 22C). DJ-X-025 also reduced the expression of inflammatory markers TNF-α and iNOS, relative to LPS alone in protein level (FIG. 16C and FIG. 16E). Moreover, DJ-X-025 also has the potential to reduce IL-6 expression in LPS-stimulated RAW 264.7 macrophages at a lower 10 UM dose (FIG. 23). Taken together, these data demonstrate that DJ-X-025 has anti-inflammatory and immunoregulatory properties on LPS-induced RAW 264.7 macrophages.

### Example 20

DJ-X-025 Improves Colon Inflammatory Parameters Associated with Colitis

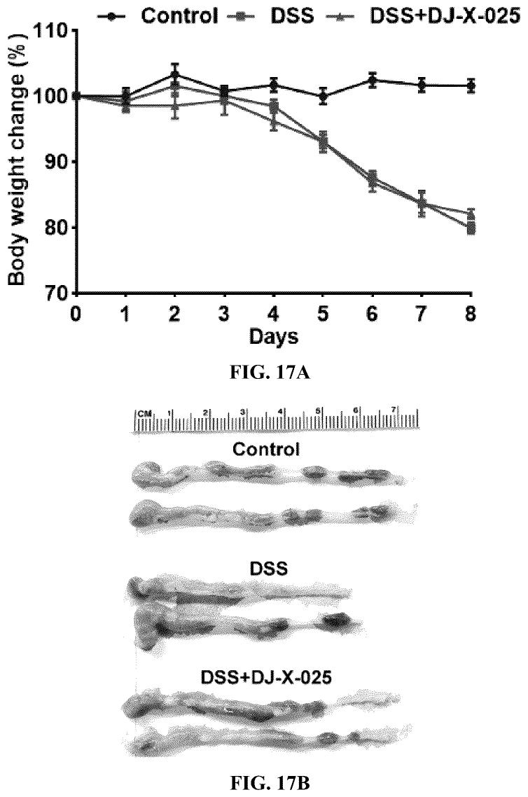

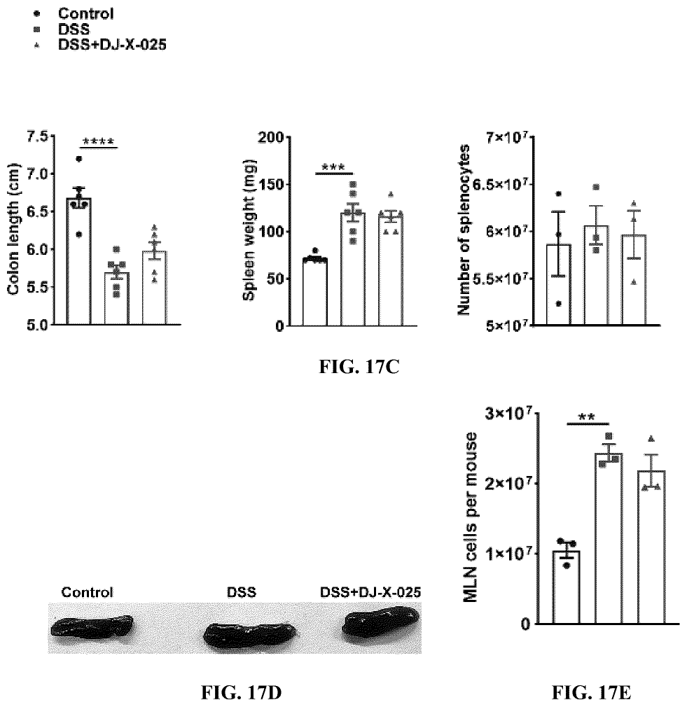

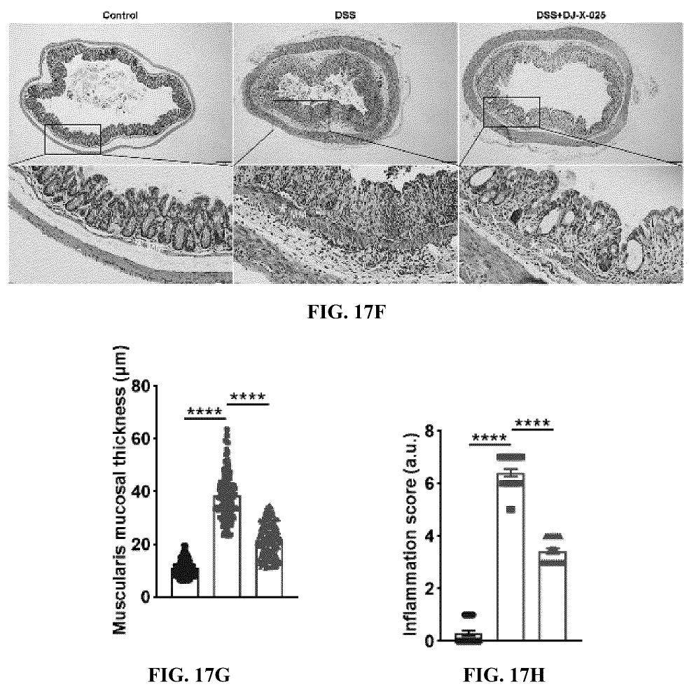

Wild-type (WT) C57BL/6 mice were treated with vehicle controls, with DSS to induce colitis, or with DSS in the presence of DJ-X-025 for seven days and compared the percent change in their body weight over this period (FIG. 17A). Although the body weight of mice treated with DSS alone or DSS with DJ-X-025 declined progressively over time relative to untreated control, mice that received DSS with DJ-X-025 showed a slight improvement in body weight from day 7 to day 8, relative to mice that received DSS alone (FIG. 17A).

At the experimental endpoint on day 8, the mice were sacrificed, colons were dissected from each mouse, and their lengths were measured. Although significant shortening of the mean colon length on DSS treatment was observed relative to untreated controls, that of mice treated with DSS plus DJ-X-025 appeared to recover somewhat, although the difference was not statistically significant (FIG. 5B). Mesenteric lymph nodes (MLNs) and spleens from each mouse and determined spleen weight and volume before dissociating the spleens and MLNs for single-cell isolation and counting the number of splenocytes and MLN cells. Relative to the control, mice in the DSS group exhibited increased spleen size and weight and increased numbers of splenocytes and MLN cells, while spleen weight and the number of MLN cells decreased slightly in mice treated with DSS and DJ-X-025 (FIG. 17C, FIG. 17D and FIG. 17E).

The colon pathology was evaluated, including the thickness of the muscularis mucosae smooth muscle layer, and determined the colon inflammatory score for the mice in each group (FIGS. 17G-17H). The colon pathology of DSS-treated mice exhibited loss of colonocytes, disrupted epithelial architecture, a hypertrophied submucosal layer, and an elevated inflammatory score, predominantly due to infiltration by various immune cells, relative to that of untreated controls (FIG. 17G and FIG. 17H). In contrast, mice treated with DSS plus DJ-X-025 displayed improved colon pathology and decreased inflammation scores relative to those of the DSS group. Interestingly, while the muscularis mucosae became thickened in the colons of mice in the DSS group, it was thinner in the colons of mice treated with DSS plus DJ-X-025 group than with DSS alone (FIG. 17F yellow stars and FIG. 17G). Together, these results demonstrate that while the modulatory effects of DJ-X-025 on inflammation are not prominent at the level of body weight, presumably due to acute colitis, at the tissue level, inflammatory cell infiltration is reduced and the inflammation score is decreased, the colon tissue architecture is improved, and the colon length is increased, suggesting that the colitis in these mice is improving.

### Example 21

### DJ-X-025 Restricted Inflammatory Myeloid Cells in the LP During Colitis

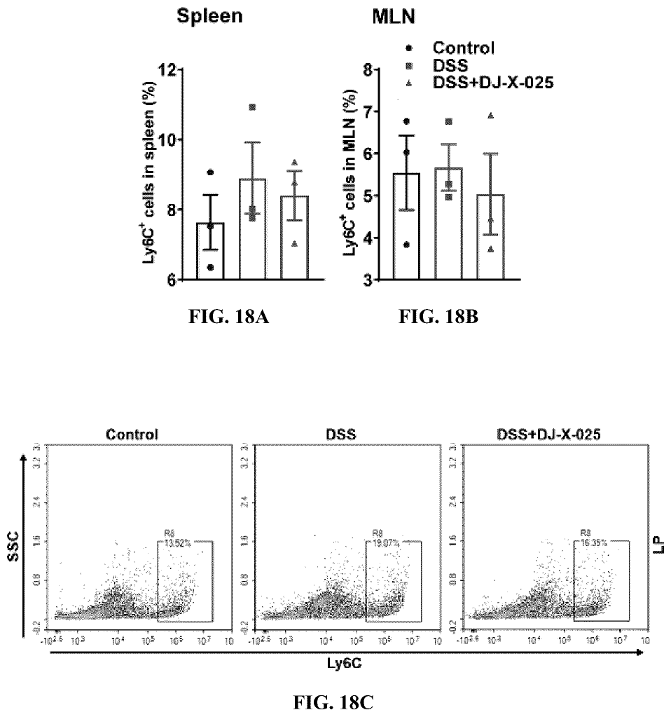

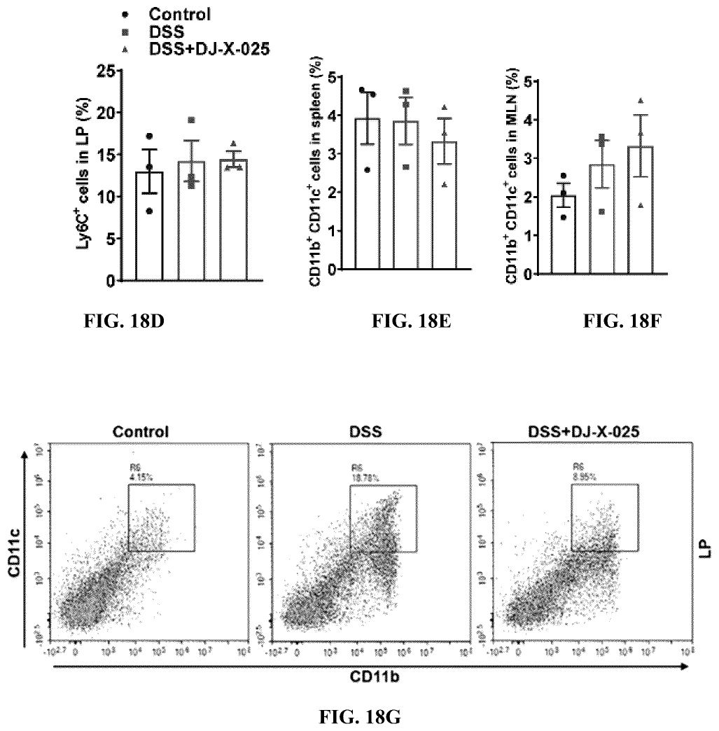

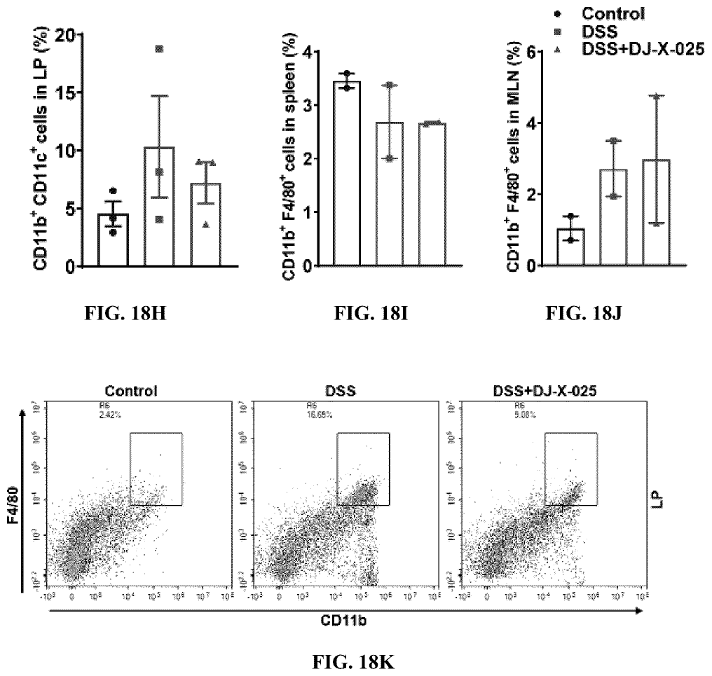

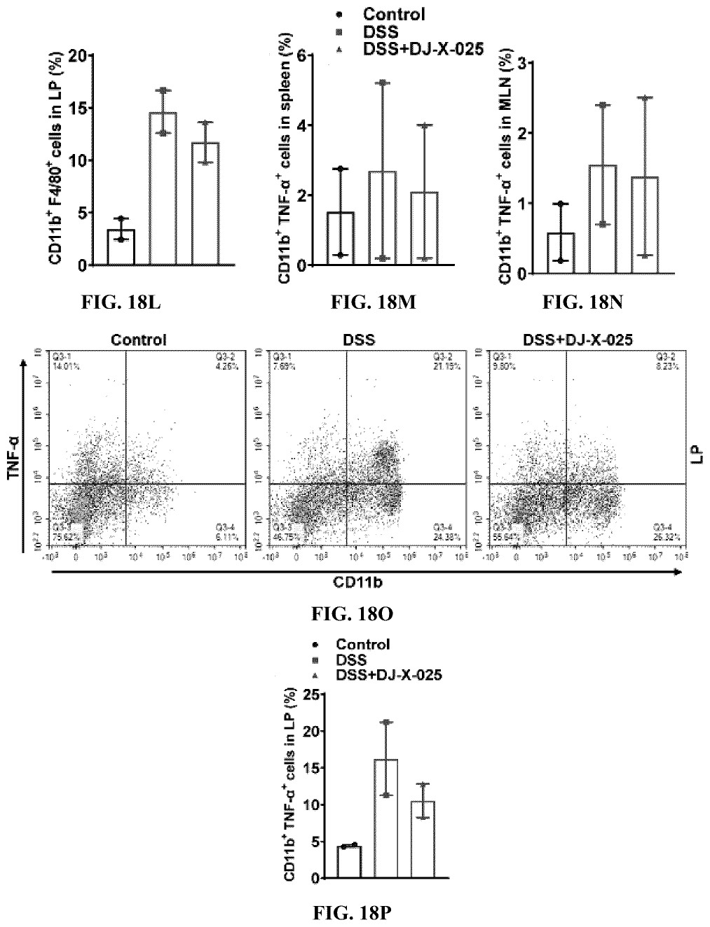

Macrophages and activated neutrophils play a key role in the progression of IBD. Dendritic cells present antigens to naive T cells, resulting in T-cell activation during colitis. It was hypothesized that a change in the frequency of mucosal and systemic macrophages, neutrophils, and/or DCs in DSS-induced colitis in the absence or presence of DJ-X-025 treatment would be observed (FIG. 18). A slight reduction in the frequency of neutrophils (Ly6C⁺ cells) in the spleen, MLNs or colon lamina propria (LP) when the mice were treated with DJ-X-025 relative to DSS alone was observed (FIGS. 18A-18D). No prominent alterations to DCs and macrophages in the spleen were observed (FIG. 18E and FIG. 18I) or MLN (FIG. 18F and FIG. 18J) among the three groups. However, a notable increase in the frequency of DCs (CD11b⁺CD11c) and macrophages (CD11b⁺F4/80) was observed in the colon LP of mice with DSS-induced colitis relative to untreated mice were isolated (FIGS. 18G-18H and FIGS. 18K-18L). Surprisingly, DJ-X-025 treatment decreased the percentage of DCs (18.78 to 8.95%) and macrophages (16.65 to 9.08%) in the colon LP relative to the mice that received DSS alone (FIG. 18G and FIG. 18K). These results demonstrate that DJ-X-025 treatment diminished the elevated numbers of DCs and macrophages in the colon LP of mice with colitis.

Inflammatory diseases, including IBD and experimental colitis, are characterized by increased expression of TNF-α by macrophages. In this study, few changes were observed among the groups in TNF-α-producing macrophage populations in the spleen (FIG. 18M) and MLN (FIG. 18N), although this may have been due to higher sample variation during this experiment. The population of CD11b⁺TNF-α macrophages significantly increased in the LP of mice treated with DSS relative to untreated controls, while the percentage of CD11b⁺TNF-α macrophages in the colon LP decreased from 21.19 to 8.23% in mice treated with DJ-X-025 relative to those receiving DSS alone (FIG. 18O and FIG. 18P). Together, these results demonstrate that DJ-X-025 strongly inhibits differentiation and/or infiltration of inflammatory myeloid cell populations in the colon LP, moderately inhibits these processes in the spleen, and may be less effective in MLNs.

### Example 22

### DJ-X-025 Differentially Modulated Activated T Cells, Th17/Tregs Response in DSS-Induced Colitis in Mice

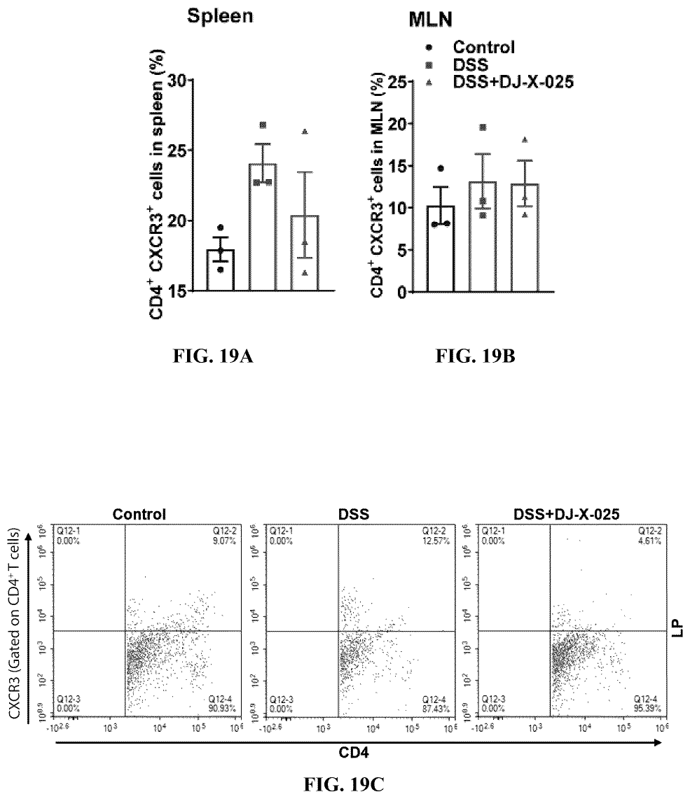

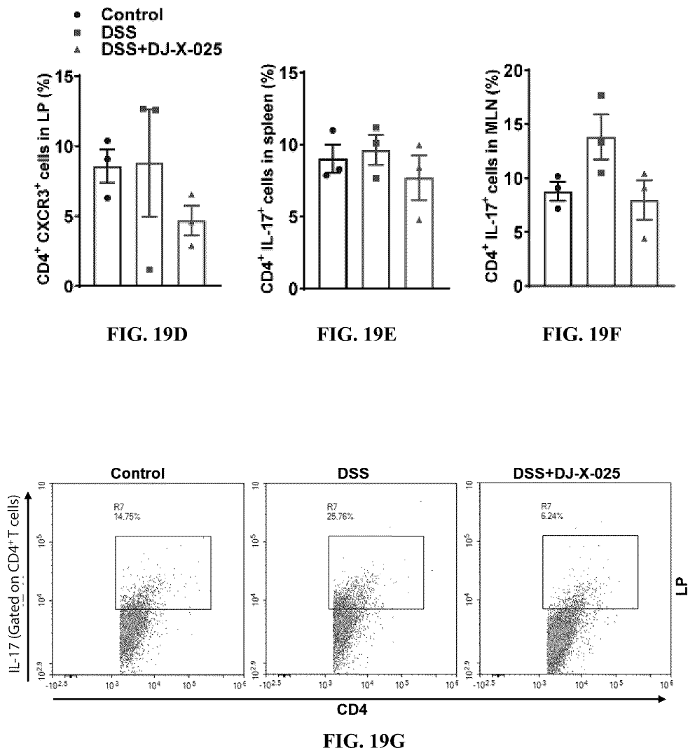

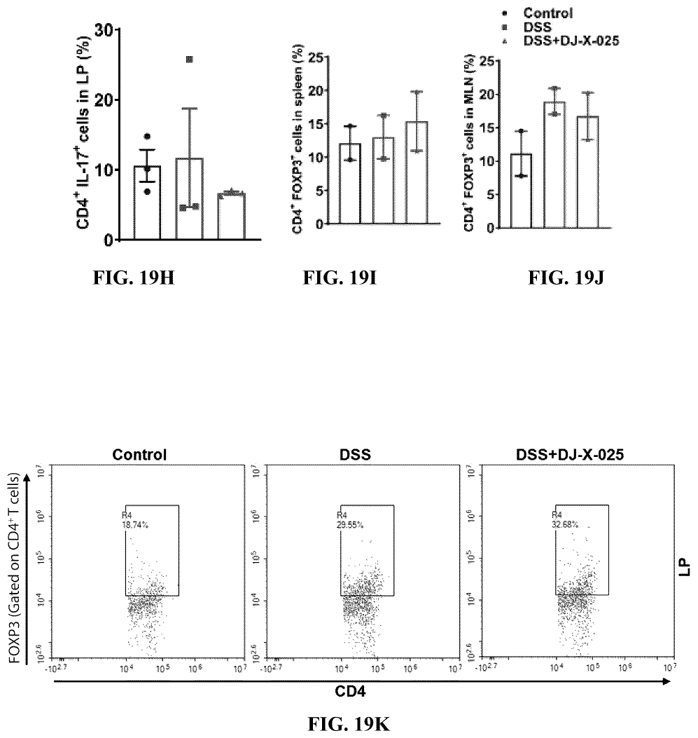

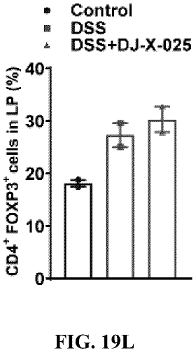

T cells are crucial for the development of IBD and the infiltration or dysregulation of Treg/Th17 cells in mice results in experimental colitis. Tregs play a crucial role in the control of intestinal inflammation and are required for the effective suppression of colitis. Thus the effects of DSS and DJ-X-025 were compared on the infiltration of activated T cells, Th17 cells, and Treg cells in the spleen, MLNs, and LP untreated mice or those treated with DSS alone or with DJ-X-025. The frequency of activated T cells expressing the C-X-C motif chemokine receptor 3 (CD4⁺CXCR3) in the spleen increased on induction of colitis with DSS, relative to that of untreated mice, but decreased after treatment with DSS plus DJ-X-025 (FIG. 19A). However, this population of T cells (CD4⁺CXCR3) was not altered in the MLNs of these mice (FIG. 19B). Moreover, the frequency of CD4⁺CXCR3 T cells was reduced in the colon LP (FIG. 19C and FIG. 19D). A similar trend of reduction in the frequency of inflammatory Th17 cells (CD4⁺IL-17) was observed in the spleens (FIG. 19E), MLNs (FIG. 19F), and LPs (FIG. 19G and FIG. 19H) of mice treated with DSS+DJ-X-025, relative to those treated with DSS alone. The number of Tregs varied a little in the spleen and MLNs (FIG. 19I and FIG. 19J) in the DSS+DJ-X-025 compared to the DSS alone. An increase in the number of Tregs in the colon LP of the mice treated with DJ-X-025 was observed relative to those given DSS alone (FIG. 19K and FIG. 19L). Taken together, these results demonstrate that DJ-X-025 treatment reduced the number of activated T cells in the spleen and colon LP and decreased the number of Th17 cells in the MLNs and colon LP, slightly altering the frequency of Tregs in the spleen and LP, thereby contributing to the suppression of colitis.

### Example 23

DJ-X-025 Treatment Attenuated Inflammation Signaling Via the p-STAT3/NF-κB Pathway and Promoted the Expression of Occludin in Mice with DSS-Induced Colitis

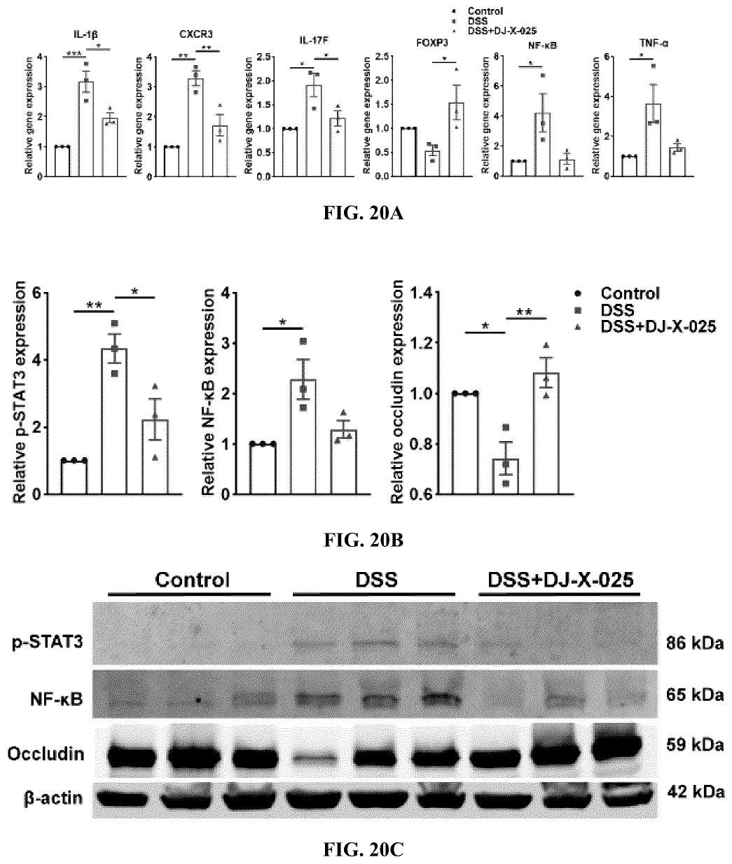

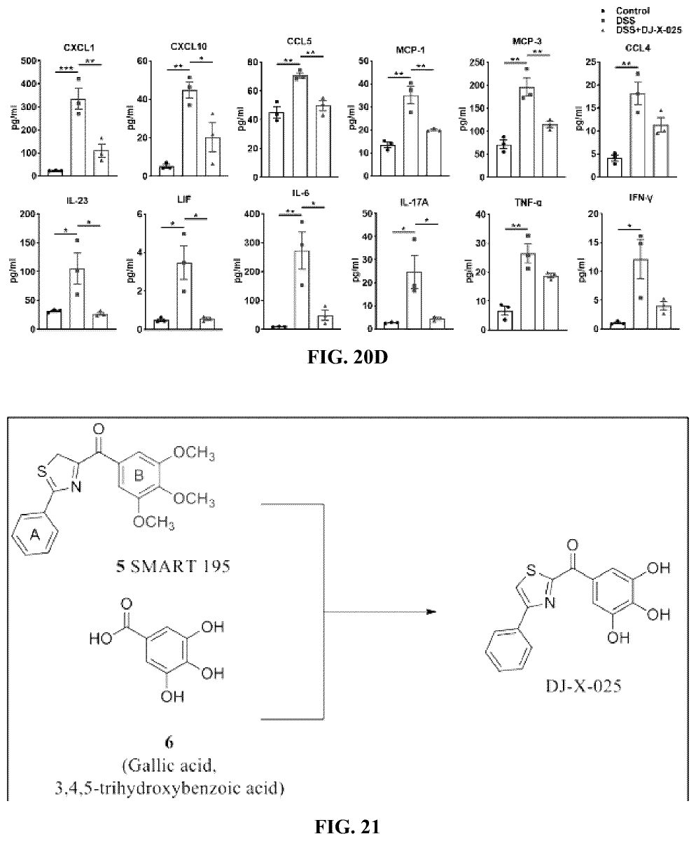

The anti-inflammatory properties of DJ-X-025 treatment were further examined by evaluating the expression of inflammatory markers at the tissue and systemic levels in mice with DSS-induced colitis (FIG. 20A, FIG. 20B, FIG. 20C, and FIG. 20D). Using RT-qPCR, expression of IL-1β, CXCR3, and IL-17 transcripts in colon tissue was significantly reduced in mice in the DSS+DJ-X-025, relative to those that received DSS alone (FIG. 20A), which support the in vitro (FIG. 16A) and in vivo findings (FIG. 19D and FIG. 19H). Foxp3 expression was also increased in mice treated with DJ-X-025, consistent with similar trends observed during flow cytometry analysis of in vivo in mice treated with DJ-X-025 after DSS induction (FIG. 19L). The expression of NF-κB and TNF-α exhibited a similar pattern of reduction in mice treated with DJ-X-025 after DSS induction (FIG. 20A), consistent with the observation that IBD patients exhibit high levels of activated NF-κB, which regulates the expression of inflammatory markers.

Elevated levels of phosphorylation on STAT3 Tyr 705 are positively correlated with disease severity in IBD patients and experimental colitis and IBD patients often experience compromised integrity of the GI tract's intestinal barrier associated with tyrosine phosphorylation, redistribution, and degradation of the tight junction protein occludin. Therefore, expression of p-STAT3, and NF-κB in colon tissue from mice treated with DJ-X-025 was measured using immunoblot analysis. A reduction in the expression of NF-κB and p-STAT3 proteins in mice that received DSS+DJ-X-025 was observed relative to those treated with DSS alone (FIG. 20B). Furthermore, disruption of tight junction proteins reduces the integrity of the epithelial barrier, leading to an increase in the severity of colitis. During colitis, the epithelial tight junctions (TJs) are dysregulated, and barrier function is diminished. Occludin is a pivotal structural protein of colon epithelial TJ. DSS decreases the expression of occludin in colon epithelial cells, and reduces occludin expression in models of intestinal inflammatory diseases, supporting its critical role in the maintenance of barrier integrity. Therefore whether DJ-X-025 can heal the colon epithelium by stabilizing the TJ occluding was evaluated. While mice treated with DSS alone exhibited decreased expression of occludin, those treated with DSS+DJ-X-025 exhibited occludin expression that resembled that of control mice (FIG. 20B), suggesting that these mice are likely to have regained their epithelial barrier. This is consistent with the observation that mice in the DSS+DJ-X-025 group exhibited a similar outcome in partially regaining their epithelial architecture as measured by histological examination of their colons (FIG. 17E). Mice treated with DSS alone exhibited increased levels of twelve systemic (serum) inflammatory cytokines and chemokines among twenty-two (tested), while mice treated with DSS and DJ-X-025 exhibited levels consistent with the baseline levels observed in control mice (FIG. 20D). DJ-X-025 also diminished other cytokine-chemokines like G-CSF, CXCL5 and IL-1β (FIG. 24). Thus, taken together, these results demonstrate the anti-inflammatory activity of compound DJ-X-025, which repressed phosphorylation of STAT3, activation of NF-κB, restored expression of occludin protein to presumably reestablish colon epithelial tight junctions, and reduced expression of inflammatory cytokines and chemokines both in colon tissue and in the circulation, thereby abrogating the signs and symptoms of colitis in mice.

The various features and processes described above may be used independently of one another, or may be combined in various ways. All possible combinations and subcombinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

What is claimed is:

1. A compound of Formula (I): 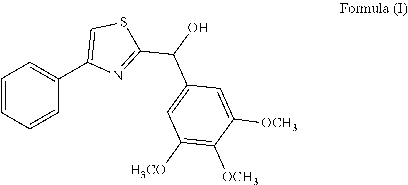 [SMILES: COc1cc(C(O)c2nc(-c3ccccc3)cs2)cc(OC)c1OC]
a prodrug thereof, or a pharmaceutically acceptable salt thereof.

2. A compound of Formula (II): 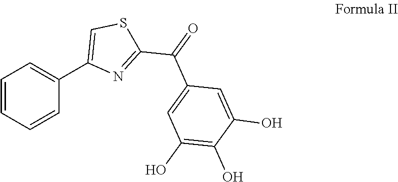 [SMILES: O=C(c1cc(O)c(O)c(O)c1)c1nc(-c2ccccc2)cs1]
a prodrug thereof, or a pharmaceutically acceptable salt thereof.

3. A method of treating an inflammatory disease comprising administering to a patient in need thereof a therapeutically effective amount of the compound of Formula (II).

4. A method of claim 3, wherein the inflammatory disease consists of rheumatoid arthritis, multiple sclerosis, systemic lupus erythematosus, type 1 diabetes, psoriasis, and inflammatory bowel disease.

5. A method of claim 3, wherein the inflammatory disease consists of Crohn's disease and ulcerative colitis.
